(19) United States
(12) Patent Application Publication
(10) Pub. No.: US 2010/0204187 A1
(43) Pub. Date: Aug. 12, 2010

(54) Purine Derivatives

(76) Inventors: Jorge Salas Solana, Barcelona (ES); Carmen Almansa Rosales, Barcelona (ES); Robert Soliva Soliva, Barcelona (ES); Montserrat Fontes Ustrell, Barcelona (ES); Marina Virgili Bernadó, Barcelona (ES); Josep Comelles Espuga, Barcelona (ES); José Javier Pastor Porras, Barcelona (ES)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/524,234

(22) PCT Filed: Jan. 23, 2008
(86) PCT No.: PCT/EP2008/050769

§ 371 Date: Mar. 12, 2010

(30) Foreign Application Priority Data
Jan. 23, 2007 (EP) 07381005.3

(51) Int. Cl. A61K 31/63; C07D 413/14; A61K 31/5377; A61P 37/06; A61P 37/00; C07D 473/00; A61K 31/52; A61K 31/551; A61K 31/541

(52) U.S. Cl. 514/157; 544/118; 514/234.2; 544/277; 514/263.22; 540/575; 514/218; 544/58.2; 514/228.5

(57) ABSTRACT

Purine derivatives of Formula (I), wherein the meanings for the various substituents are as disclosed in the description. These compounds are useful as JAK3 kinase inhibitors.

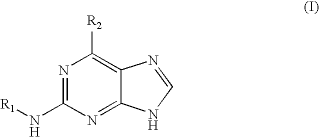

### FIELD OF THE INVENTION

The present invention relates to a new series of purine derivatives, as well as to processes for their preparation, to pharmaceutical compositions comprising them and to their use in therapy.

### BACKGROUND OF THE INVENTION

The Janus kinases (JAKs) are cytoplasmic protein tyrosine kinases that play pivotal roles in pathways that modulate cellular functions in the lympho-hematopoietic system that are critical for cell proliferation and cell survival. JAKs are involved in the initiation of cytokine-triggered signaling events by activating through tyrosine phosphorylation the signal transducers and activators of transcription (STAT) proteins. JAK/STAT signaling has been implicated in the mediation of many abnormal immune responses such as transplant rejection and autoimmune diseases, as well as in solid and hematologic malignancies such as leukemias and lymphomas and in myeloproliferative disorders, and has thus emerged as an interesting target for drug intervention.

Four members of the JAK family have been identified so far: JAK1, JAK2, JAK3 and Tyk2. Unlike JAK1, JAK2 and Tyk2, whose expression is ubiquitous, JAK3 is mainly found in hematopoietic cells. JAK3 is associated in a non-covalent manner with the γc subunit of the receptors of IL-2, IL-4, IL-7, IL-9, IL-13 and IL-15. These cytokines play an important role in the proliferation and differentiation of T lymphocytes. JAK3-deficient mouse T cells do not respond to IL-2. This cytokine is fundamental in the regulation of T lymphocytes. In this regard, it is known that antibodies directed against the IL-2 receptor are able to prevent transplant rejection. In patients with X severe combined immunodeficiency (X-SCID), very low levels of JAK3 expression as well as genetic defects in the γc subunit of the receptor have been identified, which indicates that immunosuppression is a consequence of an alteration in the JAK3 signaling pathway.

Animal studies have suggested that JAK3 not only plays a critical role in T and B lymphocyte maturation, but also that JAK3 is required to maintain lymphocyte function. Modulation of the immunological activity through this new mechanism can prove useful in the treatment of T cell proliferative disorders such as transplant rejection and autoimmune diseases.

JAK3 has also been shown to play an important role in mast cells, because antigen-induced degranulation and mediator release have been found to be substantially reduced in mast cells from JAK3 deficient mice. JAK3 deficiency does not affect mast cell proliferation nor IgE receptor expression levels. On the other hand, JAK3−/− and JAK3+/+ mast cells contain the same intracellular mediators. Therefore, JAK3 appears to be essential in the IgE-induced release of mediators in mast cells and its inhibition would be, thus, an effective treatment for allergic reactions.

In conclusion, JAK3 kinase inhibitors have been recognised as a new class of effective immunosuppresive agents useful for transplant rejection prevention and in the prevention or treatment of immune, autoimmune, inflammatory and proliferative diseases such as psoriasis, psoriatic arthritis, rheumatoid arthritis, multiple sclerosis, inflammatory bowel diseases, systemic lupus erythematosus, type I diabetes and complications from diabetes, allergic reactions and leukemia (see e.g. O'Shea J. J. et al, Nat. Rev. Drug. Discov. 2004, 3(7):555-64; Cetkovic-Cvrlje M. et al, Curr. Pharm. Des. 2004, 10(15):1767-84; Cetkovic-Cvrlje M. et al, Arch. Immunol. Ther. Exp. (Warsz), 2004, 52(2):69-82).

Accordingly, it would be desirable to provide novel compounds that are capable of inhibiting JAK/STAT signaling pathways, and in particular which are capable of inhibiting JAK3 activity, and which are good drug candidates. Compounds should exhibit good activity in in vivo pharmacological assays, good oral absorption when administered by the oral route, as well as be metabolically stable and exhibit a favourable pharmacokinetic profile. Moreover, compounds should not be toxic and exhibit few side effects.

### DESCRIPTION OF THE INVENTION

One aspect of the invention relates to a compound of formula I

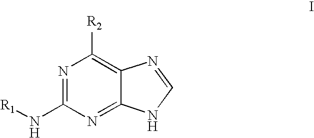

wherein:

R₁ represents phenyl or a 5- or 6-membered aromatic heterocycle bonded to the NH group through a C atom, each of which can be optionally fused to a 5- or 6-membered saturated, partially unsaturated or aromatic carbocyclic or heterocyclic ring, wherein R₁ can contain from 1 to 4 heteroatoms selected from N, O and S, wherein one or more C or S atoms of the 5- or 6-membered fused ring can be optionally oxidized forming CO, SO or SO₂ groups, and wherein R₁ can be optionally substituted with one or more R₃;

R₂ represents phenyl or a 5- or 6-membered aromatic heterocycle bonded to the purine ring through a C atom, each of which can be optionally fused to a 5- or 6-membered saturated, partially unsaturated or aromatic carbocyclic or heterocyclic ring, wherein R₂ can contain from 1 to 4 heteroatoms selected from N, O and S, wherein one or more C or S atoms of the 5- or 6-membered fused ring can be optionally oxidized forming CO, SO or SO₂ groups, and wherein R₂ can be optionally substituted with one or more R₄;

R₃ and R₄ independently represent C₁₋₄alkyl, C₂₋₄alkenyl, C₂₋₄alkynyl, halogen, —CN, —NO₂, —COR₆, —CO₂R₆, —CONR₆R₆, —OR₆, —OCOR₅, —OCONR₅R₅, —OCO₂R₅, —SR₆, —SO₂R₅, —SOR₅, —SO₂NR₆R₆, —SO₂NR₇COR₅, —NR₆R₆, —NR₇COR₆, —NR₇CONR₆R₆, —NR₇CO₂R₅, —NR₇SO₂R₅, —C(═N—OH)R₅ or Cy₁, wherein the C₁₋₄alkyl, C₂₋₄alkenyl and C₂₋₄alkynyl groups can be optionally substituted with one or more R₅ and Cy₁ can be optionally substituted with one or more R₉;

R₅ represents C₁₋₄alkyl, C₂₋₄alkenyl, C₂₋₄alkynyl or Cy₂, wherein the C₁₋₄alkyl, C₂₋₄alkenyl and C₂₋₄alkynyl groups can be optionally substituted with one or more R₁₀ and Cy₂ can be optionally substituted with one or more R₁₁;

R₆ represents hydrogen or R₅;

R₇ represents hydrogen or C₁₋₄alkyl;

R₈ represents halogen, —ON, —NO₂, —COR₁₃, —CO₂R₁₃, —CONR₁₃R₁₃, —OR₁₃, —OCOR₁₂, —OCONR₁₂R₁₂, —OCO₂R₁₂, —SR₁₃, —SO₂R₁₂, —SOR₁₂, —SO₂NR₁₃R₁₃, —SO₂NR₇COR₁₂, —NR₁₃R₁₃, —NR₇COR₁₃, —NR₇CONR₁₃R₁₃, —NR₇CO₂R₁₂, —NR₇SO₂R₁₂, —C(═N—OH)R₁₂ or Cy₂, wherein Cy₂ can be optionally substituted with one or more R₁₁;

R₉ represents C₁₋₄alkyl that can be optionally substituted with one or more R₁₀, or R₉ represents any of the meanings described for R₁₄;

R₁₀ represents halogen, —ON, —NO₂, —COR₁₆, —CO₂R₁₆, —CONR₁₆R₁₆, —OR₁₆, —OCOR₁₅, —OCONR₁₅R₁₅, —OCO₂R₁₅, —SR₁₆, —SO₂R₁₅, —SOR₁₅, —SO₂NR₁₆R₁₆, —SO₂NR₇COR₁₅, —NR₁₆R₁₆, —NR₇COR₁₆, —NR₇CONR₁₆R₁₆, —NR₇CO₂R₁₅, —NR₇SO₂R₁₅, —C(═N—OH)R₁₅ or Cy₂, wherein Cy₂ can be optionally substituted with one or more R₁₁;

R₁₁ represents C₁₋₄alkyl, haloC₁₋₄alkyl, C₁₋₄alkoxyC₁₋₄alkyl, hydroxyC₁₋₄alkyl, cyanoC₁₋₄alkyl or any of the meanings described for R₁₄;

R₁₂ represents C₁₋₄alkyl, haloC₁₋₄alkyl, C₁₋₄alkoxyC₁₋₄alkyl, hydroxyC₁₋₄alkyl, cyanoC₁₋₄alkyl, Cy₃-C₁₋₄alkyl or Cy₂, wherein Cy₂ can be optionally substituted with one or more R₁₁;

R₁₃ represents hydrogen or R₁₂;

R₁₄ represents halogen, —ON, —NO₂, —COR₁₈, —CO₂R₁₈, —CONR₁₈R₁₈, —OR₁₈, —OCOR₁₇, —OCONR₁₇R₁₇, —OCO₂R₁₇, —SR₁₈, —SO₂R₁₇, —SOR₁₇, —SO₂NR₁₈R₁₈, —SO₂NR₇COR₁₇, —NR₁₈R₁₈, —NR₇COR₁₈, —NR₇CONR₁₈R₁₈, —NR₇CO₂R₁₇, —NR₇SO₂R₁₇ or —C(═N—OH)R₁₇;

R₁₅ represents C₁₋₄alkyl, haloC₁₋₄alkyl, C₁₋₄alkoxyC₁₋₄alkyl, hydroxyC₁₋₄alkyl, cyanoC₁₋₄alkyl or Cy₂, wherein Cy₂ can be optionally substituted with one or more R₁₁;

R₁₆ represents hydrogen or R₁₅;

R₁₇ represents C₁₋₄alkyl, haloC₁₋₄alkyl, C₁₋₄alkoxyC₁₋₄alkyl, hydroxyC₁₋₄alkyl or cyanoC₁₋₄alkyl;

R₁₈ represents hydrogen or R₁₇;

or two R₁₇ groups or two R₁₈ groups on the same N atom can be bonded completing together with the N atom a saturated 5- or 6-membered ring, which can additionally contain one or two heteroatoms selected from N, S and O and which can be optionally substituted with one or more C₁₋₄alkyl groups;

Cy₁ and Cy₂ independently represent a 3- to 7-membered monocyclic or 8-to 12-membered bicyclic carbocyclic ring that can be saturated, partially unsaturated or aromatic, and which can optionally contain from 1 to 4 heteroatoms selected from N, S and O, wherein said ring can be bonded to the rest of the molecule through any available C or N atom, and wherein one or more C or S ring atoms can be optionally oxidized forming CO, SO or SO₂ groups;

Cy₃ represents a ring selected from (a)-(c):

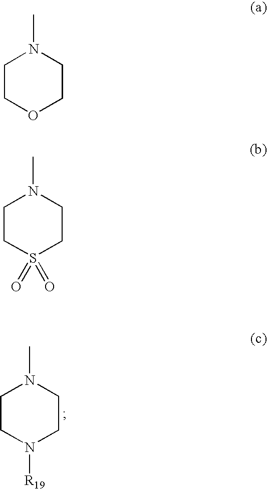

and

R₁₉ represents hydrogen or C₁₋₄alkyl.

The present invention also relates to the salts and solvates of the compounds of formula I.

Some compounds of formula I can have chiral centers that can give rise to various stereoisomers. The present invention relates to each of these stereoisomers and also mixtures thereof.

The compounds of formula I are JAK3 kinase inhibitors and therefore can be useful for the treatment or prevention of diseases mediated by this kinase.

Thus, another aspect of the invention relates to a compound of formula I

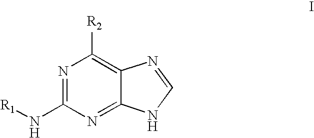

wherein:

R₁ represents phenyl or a 5- or 6-membered aromatic heterocycle bonded to the NH group through a C atom, each of which can be optionally fused to a 5- or 6-membered saturated, partially unsaturated or aromatic carbocyclic or heterocyclic ring, wherein R₁ can contain from 1 to 4 heteroatoms selected from N, O and S, wherein one or more C or S atoms of the 5- or 6-membered fused ring can be optionally oxidized forming CO, SO or SO₂ groups, and wherein R₁ can be optionally substituted with one or more R₃;

R₂ represents phenyl or a 5- or 6-membered aromatic heterocycle bonded to the purine ring through a C atom, each of which can be optionally fused to a 5- or 6-membered saturated, partially unsaturated or aromatic carbocyclic or heterocyclic ring, wherein R₂ can contain from 1 to 4 heteroatoms selected from N, O and S, wherein one or more C or S atoms of the 5- or 6-membered fused ring can be optionally oxidized forming CO, SO or SO₂ groups, and wherein R₂ can be optionally substituted with one or more R₄;

R₃ and R₄ independently represent C₁₋₄alkyl, C₂₋₄alkenyl, C₂₋₄alkynyl, halogen, —CN, —NO₂, —COR_{E}, —CO₂R₆, —CONR₆R₆, —OR₆, —OCOR₅, —OCONR₅R₅, —OCO₂R₅, —SR₆, —SO₂R₅, —SOR₅, —SO₂NR₆R₆, —SO₂NR₇COR₅, —NR₆R₆, —NR₇COR₆, —NR₇CONR₆R₆, —NR₇CO₂R₅, —NR₇SO₂R₅, —C(═N—OH)R₅ or Cy₁, wherein the C₁₋₄alkyl, C₂₋₄alkenyl and C₂₋₄alkynyl groups can be optionally substituted with one or more R₈ and Cy₁ can be optionally substituted with one or more R₉;

R₅ represents C₁₋₄alkyl, C₂₋₄alkenyl, C₂₋₄alkynyl or Cy₂, wherein the C₁₋₄alkyl, C₂₋₄alkenyl and C₂₋₄alkynyl groups can be optionally substituted with one or more R₁₀ and Cy₂ can be optionally substituted with one or more R₁₁;

R₆ represents hydrogen or R₅;

R₇ represents hydrogen or C₁₋₄alkyl;

R₈ represents halogen, —ON, —NO₂, —COR₁₃, —CO₂R₁₃, —CONR₁₃R₁₃, —OR₁₃, —OCOR₁₂, —OCONR₁₂R₁₂, —OCO₂R₁₂, —SR₁₃, —SO₂R₁₂, —SOR₁₂, —SO₂NR₁₃R₁₃, —SO₂NR₇COR₁₂, —NR₁₃R₁₃, —NR₇COR₁₃, —NR₇CONR₁₃R₁₃, —NR₇CO₂R₁₂, —NR₇SO₂R₁₂, —C(═N—OH)R₁₂ or Cy₂, wherein Cy₂ can be optionally substituted with one or more R₁₁;

R₉ represents C₁₋₄alkyl that can be optionally substituted with one or more R₁₀, or R₉ represents any of the meanings described for R₁₄;

R₁₀ represents halogen, —ON, —NO₂, —COR₁₆, —CO₂R₁₆, —CONR₁₆R₁₆, —OR₁₆, —OCOR₁₅, —OCONR₁₅R₁₅, —OCO₂R₁₅, —SR₁₆, —SO₂R₁₅, —SOR₁₅, —SO₂NR₁₆R₁₆, —SO₂NR₇COR₁₅, —NR₁₆R₁₆, —NR₇COR₁₆, —NR₇CONR₁₆R₁₆, —NR₇CO₂R₁₅, —NR₇SO₂R₁₅, —C(═N—OH)R₁₅ or Cy₂, wherein Cy₂ can be optionally substituted with one or more R₁₁;

R₁₁ represents C₁₋₄alkyl, haloC₁₋₄alkyl, C₁₋₄alkoxyC₁₋₄alkyl, hydroxyC₁₋₄alkyl, cyanoC₁₋₄alkyl or any of the meanings described for R₁₄;

R₁₂ represents C₁₋₄alkyl, haloC₁₋₄alkyl, C₁₋₄alkoxyC₁₋₄alkyl, hydroxyC₁₋₄alkyl, cyanoC₁₋₄alkyl, Cy₃-C₁₋₄alkyl or Cy₂, wherein Cy₂ can be optionally substituted with one or more R₁₁;

R₁₃ represents hydrogen or R₁₂;

R₁₄ represents halogen, —ON, —NO₂, —COR₁₈, —CO₂R₁₈, —CONR₁₈R₁₈, —OR₁₈, —OCOR₁₇, —OCONR₁₇R₁₇, —OCO₂R₁₇, —SR₁₈, —SO₂R₁₇, —SOR₁₇, —SO₂NR₁₈R₁₈, —SO₂NR₇COR₁₇, —NR₁₈R₁₈, —NR₇COR₁₈, —NR₇CONR₁₈R₁₈, —NR₇CO₂R₁₇, —NR₇SO₂R₁₇ or —C(═N—OH)R₁₇;

R₁₅ represents C₁₋₄alkyl, haloC₁₋₄alkyl, C₁₋₄alkoxyC₁₋₄alkyl, hydroxyC₁₋₄alkyl, cyanoC₁₋₄alkyl or Cy₂, wherein Cy₂ can be optionally substituted with one or more R₁₁;

R₁₆ represents hydrogen or R₁₅;

R₁₇ represents C₁₋₄alkyl, haloC₁₋₄alkyl, C₁₋₄alkoxyC₁₋₄alkyl, hydroxyC₁₋₄alkyl or cyanoC₁₋₄alkyl;

R₁₈ represents hydrogen or R₁₇;

or two R₁₇ groups or two R₁₈ groups on the same N atom can be bonded completing together with the N atom a saturated 5- or 6-membered ring, which can additionally contain one or two heteroatoms selected from N, S and O and which can be optionally substituted with one or more C₁₋₄alkyl groups;

Cy₁ and Cy₂ independently represent a 3- to 7-membered monocyclic or 8-to 12-membered bicyclic carbocyclic ring that can be saturated, partially unsaturated or aromatic, and which can optionally contain from 1 to 4 heteroatoms selected from N, S and O, wherein said ring can be bonded to the rest of the molecule through any available C or N atom, and wherein one or more C or S ring atoms can be optionally oxidized forming CO, SO or SO₂ groups;

Cy₃ represents a ring selected from (a)-(c):

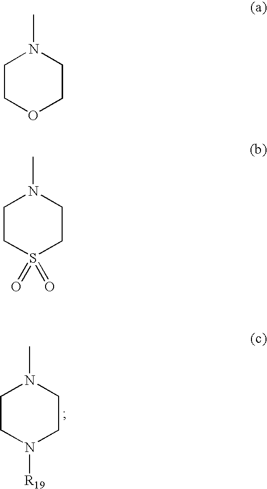

and

R₁₉ represents hydrogen or C₁₋₄alkyl,

for use in therapy.

Another aspect of this invention relates to a pharmaceutical composition, which comprises a compound of formula I or a pharmaceutically acceptable salt thereof and one or more pharmaceutically acceptable excipients.

Another aspect of the present invention relates to the use of a compound of formula I or a pharmaceutically acceptable salt thereof for the manufacture of a medicament for the treatment or prevention of diseases mediated by JAKs, particularly JAK3.

Another aspect of the present invention relates to the use of a compound of formula I or a pharmaceutically acceptable salt thereof for the manufacture of a medicament for the treatment or prevention of a disease selected from transplant rejection; immune, autoimmune or inflammatory diseases; neurodegenerative diseases; and proliferative disorders. In a preferred embodiment, the disease is selected from transplant rejection and immune, autoimmune or inflammatory diseases.

Another aspect of the present invention relates to the use of a compound of formula I or a pharmaceutically acceptable salt thereof for the manufacture of a medicament for the treatment or prevention of a disease selected from transplant rejection, rheumatoid arthritis, psoriatic arthritis, psoriasis, type I diabetes, complications from diabetes, multiple sclerosis, systemic lupus erythematosus, atopic dermatitis, mast cell-mediated allergic reactions, leukemias, lymphomas and thromboembolic and allergic complications associated with leukemias and lymphomas.

Another aspect of the present invention relates to a compound of formula I or a pharmaceutically acceptable salt thereof for the treatment or prevention of diseases mediated by JAKs, particularly JAK3.

Another aspect of the present invention relates to a compound of formula I or a pharmaceutically acceptable salt thereof for the treatment or prevention of a disease selected from transplant rejection; immune, autoimmune or inflammatory diseases; neurodegenerative diseases; and proliferative disorders. In a preferred embodiment, the disease is selected from transplant rejection and immune, autoimmune or inflammatory diseases.

Another aspect of the present invention relates to a compound of formula I or a pharmaceutically acceptable salt thereof for the treatment or prevention of a disease selected from transplant rejection, rheumatoid arthritis, psoriatic arthritis, psoriasis, type I diabetes, complications from diabetes, multiple sclerosis, systemic lupus erythematosus, atopic dermatitis, mast cell-mediated allergic reactions, leukemias, lymphomas and thromboembolic and allergic complications associated with leukemias and lymphomas.

Another aspect of the present invention relates to the use of a compound of formula I or a pharmaceutically acceptable salt thereof for the treatment or prevention of diseases mediated by JAKs, particularly JAK3.

Another aspect of the present invention relates to the use of a compound of formula I or a pharmaceutically acceptable salt thereof for the treatment or prevention of a disease selected from transplant rejection; immune, autoimmune or inflammatory diseases; neurodegenerative diseases; and proliferative disorders. In a preferred embodiment, the disease is selected from transplant rejection and immune, autoimmune or inflammatory diseases.

Another aspect of the present invention relates to the use of a compound of formula I or a pharmaceutically acceptable salt thereof for the treatment or prevention of a disease selected from transplant rejection, rheumatoid arthritis, psoriatic arthritis, psoriasis, type I diabetes, complications from diabetes, multiple sclerosis, systemic lupus erythematosus, atopic dermatitis, mast cell-mediated allergic reactions, leukemias, lymphomas and thromboembolic and allergic complications associated with leukemias and lymphomas.

Another aspect of the present invention relates to a method of treating or preventing a disease mediated by JAKs, particularly JAK3, in a subject in need thereof, especially a human being, which comprises administering to said subject an effective amount of a compound of formula I or a pharmaceutically acceptable salt thereof.

Another aspect of the present invention relates to a method of treating or preventig a disease selected from transplant rejection, immune, autoimmune or inflammatory diseases, neurodegenerative diseases, and proliferative disorders in a subject in need thereof, especially a human being, which comprises administering to said subject an effective amount of a compound of formula I or a pharmaceutically acceptable salt thereof. In a preferred embodiment, the disease is selected from transplant rejection and immune, autoimmune or inflammatory diseases.

Another aspect of the present invention relates to a method of treating or preventing a disease selected from transplant rejection, rheumatoid arthritis, psoriatic arthritis, psoriasis, type I diabetes, complications from diabetes, multiple sclerosis, systemic lupus erythematosus, atopic dermatitis, mast cell-mediated allergic reactions, leukemias, lymphomas and thromboembolic and allergic complications associated with leukemias and lymphomas in a subject in need thereof, especially a human being, which comprises administering to said subject an effective amount of a compound of formula I or a pharmaceutically acceptable salt thereof.

Another aspect of the present invention relates to a process for the preparation of a compound of formula I as defined above, which comprises:

(a) reacting a compound of formula IV with a compound of formula V

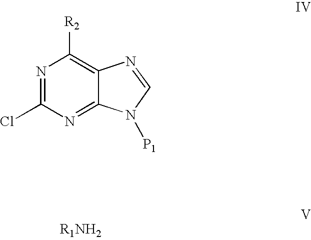

wherein R₁ and R₂ have the previously described meaning and P₁ represents an amine protecting group, followed if required by the removal of the protecting group; or
(b) reacting a compound of formula X with a compound of formula III

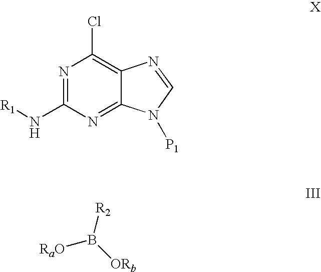

wherein R₁ and R₂ have the previously described meaning, P₁ represents an amine protecting group, and Rₐ and R_{b} represent H or C₁₋₄alkyl, or can be bonded forming together with the B and O atoms a 5- or 6-membered ring that can be optionally substituted with one or more methyl groups, followed if required by the removal of the protecting group; or
(c) reacting a compound of formula XV with a compound of formula XII

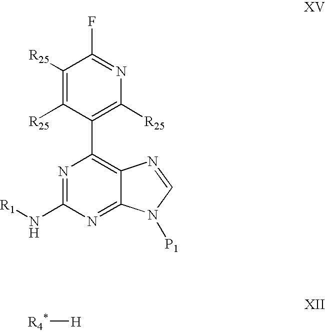

wherein R₄* represents —NR₆R₆ or Cy₁ bonded through a N atom to the pyridine ring, each R₂₅ independently represents hydrogen, halogen, C₁₋₄alkyl, C₁₋₄alkoxy, haloC₁₋₄alkoxy or —SC₁₋₄alky, P₁ represents an amine protecting group and R₁, Cy₁ and R₆ have the meaning previously described, followed if required by the removal of the protecting group; or
(d) converting, in one or a plurality of steps, a compound of formula I into another compound of formula I.

In the above definitions, the term C₁₋₄alkyl, as a group or part of a group, means a straight or branched alkyl chain which contains from 1 to 4 carbon atoms and includes the groups methyl, ethyl, propyl, isopropyl, butyl, isobutyl, sec-butyl and tert-butyl.

A C₂₋₄alkenyl group means a straight or branched alkyl chain which contains from 2 to 4 C atoms, and also contains one or two double bonds. Examples include the groups ethenyl, 1-propenyl, 2-propenyl, isopropenyl, 1-butenyl, 2-butenyl, 3-butenyl and 1,3-butadienyl.

A C₂₋₄alkynyl group means straight or branched alkyl chain which contains from 2 to 4 C atoms, and also contains one or two triple bonds. Examples include the groups ethynyl, 1-propynyl, 2-propynyl, 1-butynyl, 2-butynyl, 3-butynyl and 1,3-butadiynyl.

A C₁₋₄alkoxy group, as a group or part of a group, means a group —OC₁₋₄alkyl, wherein the C₁₋₄alkyl moiety has the same meaning as previously described. Examples include methoxy, ethoxy, propoxy, isopropoxy, butoxy, isobutoxy, sec-butoxy and tert-butoxy.

A halogen group or its abbreviation halo means fluoro, chloro, bromo or iodo.

A C₁₋₄alkoxyC₁₋₄alkyl group means a group resulting from the replacement of one or more hydrogen atoms from a C₁₋₄alkyl group with one or more C₁₋₄alkoxy groups, which can be the same or different. Examples include, among others, the groups methoxymethyl, ethoxymethyl, propoxymethyl, isopropoxymethyl, butoxymethyl, isobutoxymethyl, sec-butoxymethyl, tert-butoxymethyl, dimethoxymethyl, 1-methoxyethyl, 2-methoxyethyl, 2-ethoxyethyl, 1,2-diethoxyethyl, 1-butoxyethyl, 2-sec-butoxyethyl, 3-methoxypropyl, 2-butoxypropyl, 1-methoxy-2-ethoxypropyl, 3-tert-butoxypropyl and 4-methoxybutyl.

A haloC₁₋₄alkyl group means a group resulting from the replacement of one or more hydrogen atoms from a C₁₋₄alkyl group with one or more halogen atoms (i.e. fluoro, chloro, bromo or iodo), which can be the same or different. Examples include, among others, the groups trifluoromethyl, fluoromethyl, 1-chloroethyl, 2-chloroethyl, 1-fluoroethyl, 2-fluoroethyl, 2-bromoethyl, 2-iodoethyl, 2,2,2-trifluoroethyl, pentafluoroethyl, 3-fluoropropyl, 3-chloropropyl, 2,2,3,3-tetrafluoropropyl, 2,2,3,3,3-pentafluoropropyl, heptafluoropropyl, 4-fluorobutyl and nonafluorobutyl.

A haloC₁₋₄alkoxy group means a group resulting from the replacement of one or more hydrogen atoms from a C₁₋₄alkoxy group with one or more halogen atoms (i.e. fluoro, chloro, bromo or iodo), which can be the same or different. Examples include, among others, the groups trifluoromethoxy, fluoromethoxy, 1-chloroethoxy, 2-chloroethoxy, 1-fluoroethoxy, 2-fluoroethoxy, 2-bromoethoxy, 2-iodoethoxy, 2,2,2-trifluoroethoxy, pentafluoroethoxy, 3-fluoropropoxy, 3-chloropropoxy, 2,2,3,3-tetrafluoropropoxy, 2,2,3,3,3-pentafluoropropoxy, heptafluoropropoxy, 4-fluorobutoxy and nonafluorobutoxy.

A hydroxyC₁₋₄alkyl group means a group resulting from the replacement of one or more hydrogen atoms from a C₁₋₄alkyl group with one or more hydroxy groups. Examples include, among others, the groups hydroxymethyl, 1-hydroxyethyl, 2-hydroxyethyl, 1,2-dihydroxyethyl, 3-hydroxypropyl, 2-hydroxypropyl, 1-hydroxypropyl, 2,3-dihydroxypropyl, 4-hydroxybutyl, 3-hydroxybutyl, 2-hydroxybutyl and 1-hydroxybutyl.

A cyanoC₁₋₄alkyl group means a group resulting from the replacement of one or more hydrogen atoms from a C₁₋₄alkyl group with one or more cyano groups. Examples include, among others, the groups cyanomethyl, dicyanomethyl, 1-cyanoethyl, 2-cyanoethyl, 3-cyanopropyl, 2,3-dicyanopropyl and 4-cyanobutyl.

A Cy₃-C₁₋₄alkyl group means a group resulting from the replacement of one hydrogen atom from a C₁₋₄alkyl group with one Cy₃ group. Examples include, among others, the groups (morpholin-4-yl)methyl, 2-(morpholin-4-yl)ethyl, 3-(morpholin-4-yl)propyl, 4-(morpholin-4-yl)butyl, (piperazin-1-yl)methyl, (4-methylpiperazin-1-yl)methyl, 2-(4-methylpiperazin-1-yl)ethyl, 3-(4-methylpiperazin-1-yl)propyl, 4-(4-methylpiperazin-1-yl)butyl, (4-ethylpiperazin-1-yl)methyl, (4-propylpiperazin-1-yl)methyl, (4-butylpiperazin-1-yl)methyl, (1,1-dioxothiomorpholin-4-yl)methyl, 2-(1,1-dioxotiomorpholin-4-yl)ethyl, 3-(1,1-dioxothiomorpholin-4-yl)propyl and 4-(1,1-dioxothiomorpholin-4-yl)butyl.

A Cy₂, C₁₋₄alkyl group means a group resulting from the replacement of one hydrogen atom from a C₁₋₄alkyl group with one Cy₂, group as defined below.

The term Cy₁ or Cy₂ refers to a 3- to 7-membered monocyclic or a 8- to 12-membered bicyclic carbocyclic ring that can be saturated, partially unsaturated or aromatic, and which optionally contains from 1 to 4 heteroatoms selected from N, S and O. When Cy₁ or Cy₂ are saturated or partially unsaturated, one or more C or S ring atoms can be optionally oxidized forming CO, SO or SO₂ groups. Cy₁ and Cy₂ can be optionally substituted as disclosed above in the definition of a compound of formula I; if substituted, the substituents can be the same or different and can be placed on any available position. Cy₁ and Cy₂ can be bonded to the rest of the molecule through any available carbon or nitrogen atom. Examples of Cy₁ and Cy₂ include, among others, cyclopropyl, cyclobutyl, cyclopentyl, cyclohexyl, cycloheptyl, azetidinyl, aziridinyl, oxyranyl, oxetanyl, imidazolidinyl, isothiazolidinyl, isoxazolidinyl, oxazolidinyl, pyrazolidinyl, pyrrolidinyl, thiazolidinyl, dioxanyl, morpholinyl, thiomorpholinyl, 1,1-dioxothiomorpholinyl, piperazinyl, homopiperazinyl, piperidinyl, pyranyl, tetrahydropyranyl, azepinyl, oxazinyl, oxazolinyl, pyrrolinyl, thiazolinyl, pyrazolinyl, imidazolinyl, isoxazolinyl, isothiazolinyl, cyclobutanonyl, cyclopentanonyl, cyclohexanonyl, cycloheptanonyl, 2-oxo-pyrrolidinyl, 2-oxo-piperidinyl, 4-oxo-piperidinyl, 2(1H)-pyridonyl, 2(1H)-pyrazinonyl, 2(1H)-pyrimidinonyl, 3(2H)-pyridazinonyl, azetidinonyl, imidazolidinonyl, oxazolidinonyl, phenyl, naphthyl, thienyl, furyl, pyrrolyl, thiazolyl, isothiazolyl, oxazolyl, isoxazolyl, imidazolyl, pyrazolyl, 1,2,3-triazolyl, 1,2,4-triazolyl, tetrazolyl, 1,3,4-oxadiazolyl, 1,3,4-thiadiazolyl, 1,2,4-oxadiazolyl, 1,2,4-thiadiazolyl, pyridyl, pyrazinyl, pyrimidinyl, pyridazinyl, benzimidazolyl, benzooxazolyl, benzofuranyl, isobenzofuranyl, indolyl, isoindolyl, benzothiophenyl, benzothiazolyl, quinolinyl, isoquinolinyl, phtalazinyl, quinazolinyl, quinoxalinyl, cinolinyl, naphthyridinyl, indazolyl, imidazopyridinyl, pyrrolopyridinyl, thienopyridinyl, imidazopyrimidinyl, imidazopyrazinyl, imidazopyridazinyl, pyrazolopyrazinyl, pyrazolopyridinyl, pyrazolopyrimidinyl, benzo[1,3]dioxolyl, phtalimidyl, 1-oxo-1,3-dihydroisobenzofuranyl, 1,3-dioxo-1,3-dihydroisobenzofuranyl, 2-oxo-2,3-dihydro-1H-indolyl, 1-oxo-2,3-dihydro-1H-isoindolyl, 1,2,3,4-tetrahydroquinolinyl, 1,2,3,4-tetrahydroisoquinolinyl, 1-oxo-1,2,3,4-tetrahydroisoquinolinyl, 1-oxo-1,2-dihydroisoquinolinyl and 4-oxo-3,4-dihydroquinazolinyl.

In a compound of formula I R₁ and R₂ represent a phenyl group or a 5- or 6-membered aromatic heterocycle which is bonded through a C atom to the NH group, in the case of R₁, and to the purine ring, in the case of R₂. Both the phenyl group and the 5- or 6-membered aromatic heterocycle can be optionally fused to a 5- or 6-membered carbocyclic or heterocyclic ring that can be saturated, partially unsaturated or aromatic. The R₁ and R₂ groups can thus be either monocyclic or bicyclic and can contain from 1 to 4 heteroatoms in total selected from N, O and S. When the second ring, that is, the fused 5- or 6-membered carbocyclic or heterocyclic ring, is saturated or partially unsaturated, one or more C or S atoms of said ring can be optionally oxidized forming CO, SO or SO₂ groups. R₁ can be optionally substituted with one or more R₃ and R₂ can be optionally substituted with one or more R₄, as indicated above in the definition of a compound of formula I. Each R₃ and each R₄ is independently selected from the list of possible meanings for said groups indicated in the definition of a compound of formula I. If present, the substituents on R₁ or R₂ can be placed in any available position. Examples of R₁ and R₂ include, among others, phenyl, naphthyl, thienyl, furyl, pyrrolyl, thiazolyl, isothiazolyl, oxazolyl, isoxazolyl, imidazolyl, pyrazolyl, 1,2,3-triazolyl, 1,2,4-triazolyl, tetrazolyl, 1,3,4-oxadiazolyl, 1,3,4-thiadiazolyl, 1,2,4-oxadiazolyl, 1,2,4-thiadiazolyl, pyridyl, pyrazinyl, pyrimidinyl, pyridazinyl, benzimidazolyl, benzooxazolyl, benzofuranyl, isobenzofuranyl, indolyl, isoindolyl, benzothiophenyl, benzothiazolyl, quinolinyl, isoquinolinyl, phtalazinyl, quinazolinyl, quinoxalinyl, cinolinyl, naphthyridinyl, indazolyl, imidazopyridinyl, pyrrolopyridinyl, thienopyridinyl, imidazopyrimidinyl, imidazopyrazinyl, imidazopyridazinyl, pyrazolopyrazinyl, pyrazolopyridinyl, pyrazolopyrimidinyl, benzo[1,3]dioxolyl, phtalimidyl, 1-oxo-1,3-dihydroisobenzofuranyl, 1,3-dioxo-1,3-dihydroisobenzofuranyl, 2-oxo-2,3-dihydro-1H-indolyl, 1-oxo-2,3-dihydro-1H-isoindolyl, 1,2,3,4-tetrahydroquinolinyl, 1,2,3,4-tetrahydroisoquinolinyl, 1-oxo-1,2,3,4-tetrahydroisoquinolinyl, 1-oxo-1,2-dihydroisoquinolinyl and 4-oxo-3,4-dihydroquinazolinyl.

In the above definitions of Cy₁, Cy₂, R₁ and R₂, when the examples listed refer to a bicycle in general terms, all possible dispositions of the atoms are included. Thus, for example, the term pyrazolopyridinyl can include groups such as 1H-pyrazolo[3,4-b]pyridinyl, 1H-pyrazolo[1,5-a]pyridinyl, 1H-pyrazolo[3,4-c]pyridinyl, 1H-pyrazolo[4,3-c]pyridinyl and 1H-pyrazolo[4,3-b]pyridinyl; the term imidazopyrazinyl can include groups such as 1H-imidazo[4,5-b]pyrazinyl, imidazo[1,2-a]pyrazinyl and imidazo[1,5-a]pyrazinyl; and the term pyrazolopyrimidinyl can include groups such as 1H-pyrazolo[3,4-d]pyrimidinyl, 1H-pyrazolo[4,3-d]pyrimidinyl, pyrazolo[1,5-a]pyrimidinyl and pyrazolo[1,5-c]pyrimidinyl.

When in the definitions used throughout the present specification for cyclic groups the examples given refer to a radical of a ring in general terms, for example pyridyl, thienyl or indolyl, all possible positions of attachment are included, unless any limitation is mentioned in the definition of the corresponding group, for example that the ring is bonded through a C atom in R₁ and R₂, in which case such limitation applies. Thus for example, in the definitions for Cy₁ and Cy₂, which do not include any limitation with regard to the position of attachment, the term pyridyl includes 2-pyridyl, 3-pyridyl and 4-pyridyl; thienyl includes 2-thienyl and 3-thienyl; and indolyl includes 1-indolyl, 2-indolyl, 3-indolyl, 4-indolyl, 5-indolyl, 6-indolyl and 7-indolyl.

The expression “optionally substituted with one or more” means that a group can be substituted with one or more, preferably with 1, 2, 3 or 4 substituents, more preferably with 1, 2 or 3 substituents, and still more preferably 1 or 2 substituents, provided that said group has enough positions susceptible of being substituted. The substituents can be the same or different and can be placed on any available position.

When in the definition of a substituent two or more groups with the same numbering are indicated (e.g. —NR₇CONR₆R₆, —NR₁₆R₁₆, —CONR₁₈R₁₈, etc.), this does not mean that they must be the same. Each of them is independently selected from the list of possible meanings given for said group, and therefore they can be the same or different.

The invention thus relates to the compounds of formula I as defined above.

In another embodiment, the invention relates to the compounds of formula I wherein R₁ represents phenyl or pyridyl, which can be optionally fused to a 5- or 6-membered saturated, partially unsaturated or aromatic carbocyclic or heterocyclic ring, wherein R₁ can contain from 1 to 4 heteroatoms selected from N, O and S, wherein one or more C or S atoms of the 5- or 6-membered fused ring can be optionally oxidized forming CO, SO or SO₂ groups, and wherein R₁ can be optionally substituted with one or more R₃.

In another embodiment, the invention relates to the compounds of formula I wherein R₁ represents phenyl, pyridyl or a ring of formula R₁ₐ,

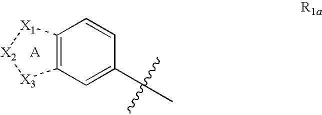

wherein in ring A X₁, X₂ and X₃ are selected from C, N, O and S and the dashed lines represent single or double bonds, wherein one or two C or S atoms of ring A can be optionally oxidized forming CO, SO or SO₂ groups, and wherein the phenyl, pyridyl and R₁ₐ groups can be optionally substituted with one or more R₃.

In another embodiment, the invention relates to the compounds of formula I wherein R₁ represents phenyl, 3-pyridyl, 4-pyridyl or a ring of formula R₁ₐ, each of which can be optionally substituted with one or more R₃.

In another embodiment, the invention relates to the compounds of formula I wherein R₁ represents phenyl, pyridyl, benzo[1,3]dioxolyl or benzooxazolyl, each of which can be optionally substituted with one or more R₃.

In another embodiment, the invention relates to the compounds of formula I wherein R₁ represents phenyl, 3-pyridyl, 4-pyridyl, 5-benzo[1,3]dioxolyl or 6-benzooxazolyl, each of which can be optionally substituted with one or more R₃.

In another embodiment, the invention relates to the compounds of formula I wherein R₁ represents phenyl optionally substituted with one or more R₃.

In another embodiment, the invention relates to the compounds of formula I wherein R₁ represents phenyl substituted with one or more R₃.

In another embodiment, the invention relates to the compounds of formula I wherein R₁ represents phenyl substituted with one, two or three R₃.

In another embodiment, the invention relates to the compounds of formula I wherein R₁ represents phenyl substituted with one or two R₃.

In another embodiment, the invention relates to the compounds of formula I wherein R₁ represents phenyl substituted with one or two R₃, which are placed at positions 3, 4 and/or 5 of the phenyl ring.

In another embodiment, the invention relates to the compounds of formula I wherein each R₃ independently represents C₁₋₄alkyl, halogen, —CN, —COR₆, —CO₂R₆, —CONR₆R₆, —OR₆, —SR₆, —SO₂R₅, —SO₂NR₆R₆, —SO₂NR₇COR₅, —NR₆R₆, —NR₇COR₆, —NR₇CONR₆R₆, —NR₇SO₂R₅ or Cy₁, wherein the C₁₋₄alkyl group can be optionally substituted with one or more R₈ and Cy₁ can be optionally substituted with one or more R₉.

In another embodiment, the invention relates to the compounds of formula I wherein each R₃ independently represents C₁₋₄alkyl, halogen, —CN, —OR₆, —SO₂R₅, —SO₂NR₆R₆, —SO₂NR₇COR₅, —NR₆R₆, —NR₇COR₆, —NR₇SO₂R₅ or Cy₁, wherein the C₁₋₄alkyl group can be optionally substituted with one or more R₈ and Cy₁ can be optionally substituted with one or more R₉.

In another embodiment, the invention relates to the compounds of formula I wherein each R₃ independently represents C₁₋₄alkyl, halogen, haloC₁₋₄alkyl, hydroxyC₁₋₄alkyl, C₁₋₄alkoxyC₁₋₄alkyl, —CN, —OR₆, —SO₂R₅, —SO₂NR₆R₆, —SO₂NR₇COR₅, —NR₆R₆, —NR₇COR₆, —NR₇SO₂R₅ or Cy₁, wherein Cy₁ can be optionally substituted with one or more R₉.

In another embodiment, the invention relates to the compounds of formula I wherein Cy₁ in R₃ is Cy₁ₐ and Cy₁ₐ represents a 5- or 6-membered saturated monocyclic heterocycle which contains 1 or 2 heteroatoms selected from N, S and O, wherein said ring can be bonded to the rest of the molecule through any available C or N atom, and wherein one or more C or S ring atoms can be optionally oxidized forming CO, SO or SO₂ groups, wherein said Cy₁ₐ can be optionally substituted with one or more R₉.

In another embodiment, the invention relates to the compounds of formula I wherein Cy₁ in R₃ is Cy_{1c} and Cy_{1c} represents a 5- or 6-membered saturated monocyclic heterocycle which contains 1 or 2 heteroatoms selected from N, S and O with the proviso that it contains at least 1 N atom, wherein said ring is bonded to the rest of the molecule through a N atom, wherein one or more C or S ring atoms can be optionally oxidized forming CO, SO or SO₂ groups, and wherein said Cy_{1c} can be optionally substituted with one or more R₉.

In another embodiment, the invention relates to the compounds of formula I wherein Cy₁ in R₃ represents a ring selected from (i)-(iii):

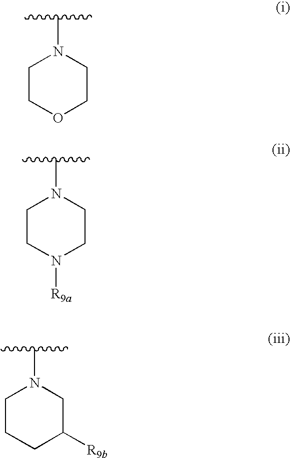

wherein R₉ₐ represents hydrogen or C₁₋₄alkyl, and R_{9b} represents hydrogen, C₁₋₄alkyl or hydroxy.

In another embodiment, the invention relates to the compounds of formula I wherein each R₃ independently represents C₁₋₄alkyl, halogen, —OR₆, —SO₂NR₆R₆, —SO₂NR₇COR₅, —NR₆R₆, —NR₇COR₆ or Cy₁ₐ, wherein the C₁₋₄alkyl group can be optionally substituted with one or more R₈ and Cy₁ₐ can be optionally substituted with one or more R₉.

In another embodiment, the invention relates to the compounds of formula I wherein each R₃ independently represents C₁₋₄alkyl, halogen, hydroxyC₁₋₄alkyl, C₁₋₄alkoxyC₁₋₄alkyl, —OR₆, Cy₂ₐC₁₋₄alkyl, —SO₂NR₆R₆, —SO₂NR₇COR₅, —NR₆R₆, —NR₇COR₆ or Cy_{1c}, wherein Cy_{1c} can be optionally substituted with one or more R₉, and wherein Cy₂ₐ represents a 5- or 6-membered saturated monocyclic heterocycle which contains 1 or 2 heteroatoms selected from N, S and O and which can be bonded to the rest of the molecule through any available C or N atom, wherein one or more C or S ring atoms can be optionally oxidized forming CO, SO or SO₂ groups, and wherein said Cy₂ₐ can be optionally substituted with one or more R₁₁.

In another embodiment, the invention relates to the compounds of formula I wherein each R₃ independently represents C₁₋₄alkyl, halogen, hydroxyC₁₋₄alkyl, C₁₋₄alkoxyC₁₋₄alkyl, —OR₆, Cy₂ₐC₁₋₄alkyl, —SO₂NR₆R₆, —SO₂NR₇COR₅, —NR₆R₆, —NR₇COR₆ or a ring of formula (i)-(iii), wherein Cy₂ₐ can be optionally substituted with one or more R₁₁.

In another embodiment, the invention relates to the compounds of formula I wherein R₆ in R₃ represents hydrogen or R₅ and R₅ represents C₁₋₄alkyl optionally substituted with one or more R₁₀.

In another embodiment, the invention relates to the compounds of formula I wherein R₆ in R₃ represents hydrogen or R₅ and R₅ represents C₁₋₄alkyl, hydroxyC₁₋₄alkyl or C₁₋₄alkoxyC₁₋₄alkyl.

In another embodiment, the invention relates to the compounds of formula I wherein:

R₁ represents phenyl substituted with one or more R₃;

each R₃ independently represents C₁₋₄alkyl, halogen, haloC₁₋₄alkyl, hydroxyC₁₋₄alkyl, C₁₋₄alkoxyC₁₋₄alkyl, —CN, —OR₆, —SO₂R₅, —SO₂NR₆R₆, —SO₂NR₇COR₅, —NR₆R₆, —NR₇COR₆, —NR₇SO₂R₅ or Cy₁ₐ, wherein Cy₁ₐ can be optionally substituted with one or more R₉; and

Cy₁ₐ represents a 5- or 6-membered saturated monocyclic heterocycle which contains 1 or 2 heteroatoms selected from N, S and O, wherein said ring can be bonded to the rest of the molecule through any available C or N atom, and wherein one or more C or S ring atoms can be optionally oxidized forming CO, SO or SO₂ groups.

In another embodiment, the invention relates to the compounds of formula I wherein:

R₁ represents a ring of formula R_{1b}:

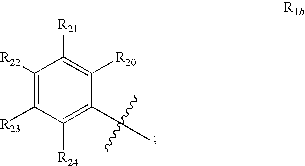

one of R₂₁, R₂₂ and R₂₃ represents hydroxyC₁₋₄alkyl, —CN, —OR₆, —SO₂NR₆R₆, —NR₇COR₆, —NR₇SO₂R₅ or Cy₁ₐ, wherein Cy₁ₐ can be optionally substituted with one or more R₉; and

the remainder of R₂₁, R₂₂ and R₂₃ as well as R₂₀ and R₂₄ are independently selected from hydrogen, C₁₋₄alkyl, halogen and C₁₋₄alkoxy.

In another embodiment, the invention relates to the compounds of formula I wherein:

R₁ represents phenyl substituted with one or more, preferably one or two R₃; and

each R₃ independently represents C₁₋₄alkyl, halogen, —OR₆, —SO₂NR₆R₆, —SO₂NR₇COR₅, —NR₆R₆, —NR₇COR₆ or Cy₁ₐ, wherein the C₁₋₄alkyl group can be optionally substituted with one or more R₈ and Cy₁ₐ can be optionally substituted with one or more R₉.

In another embodiment, the invention relates to the compounds of formula I wherein:

R₁ represents phenyl substituted with one or more, preferably one or two

R₃; and

each R₃ independently represents C₁₋₄alkyl, halogen, hydroxyC₁₋₄alkyl, C₁₋₄alkoxyC₁₋₄alkyl, —OR₆, Cy₂ₐC₁₋₄alkyl, —SO₂NR₆R₆, —SO₂NR₇COR₅, —NR₆R₆, —NR₇COR₆ or Cy_{1c}, wherein Cy_{1c} can be optionally substituted with one or more R₉, and wherein Cy₂ₐ can be optionally substituted with one or more R₁₁.

In another embodiment, the invention relates to the compounds of formula I wherein:

R₁ represents phenyl substituted with one or two R₃, which are placed at positions 3, 4 and/or 5 of the phenyl ring; and

each R₃ independently represents C₁₋₄alkyl, halogen, hydroxyC₁₋₄alkyl,

C₁₋₄alkoxyC₁₋₄alkyl, —OR₆, Cy₂ₐC₁₋₄alkyl, —SO₂NR₆R₆, —SO₂NR₇COR₅, —NR₆R₆, —NR₇COR₆ or Cy_{1c}, wherein Cy_{1c} can be optionally substituted with one or more R₉, and wherein Cy₂ₐ can be optionally substituted with one or more R₁₁.

In another embodiment, the invention relates to the compounds of formula I wherein:

R₁ represents phenyl substituted with one or more, preferably one or two R₃; and

each R₃ independently represents C₁₋₄alkyl, halogen, hydroxyC₁₋₄alkyl, C₁₋₄alkoxyC₁₋₄alkyl, —OR₆, Cy₂ₐC₁₋₄alkyl, —SO₂NR₆R₆, —SO₂NR₇COR₅, —NR₆R₆, —NR₇COR₆ or a ring of formula (i)-(iii), wherein Cy₂ₐ can be optionally substituted with one or more R₁₁.

In another embodiment, the invention relates to the compounds of formula I wherein:

R₁ represents phenyl substituted with one or two R₃, which are placed at positions 3, 4 and/or 5 of the phenyl ring; and

each R₃ independently represents C₁₋₄alkyl, halogen, hydroxyC₁₋₄alkyl, C₁₋₄alkoxyC₁₋₄alkyl, —OR₆, Cy₂ₐC₁₋₄alkyl, —SO₂NR₆R₆, —SO₂NR₇COR₅, —NR₆R₆, —NR₇COR₆ or a ring of formula (i)-(iii), wherein Cy₂ₐ can be optionally substituted with one or more R₁₁.

In another embodiment, the invention relates to the compounds of formula I wherein:

R₁ represents a ring of formula R_{1c}:

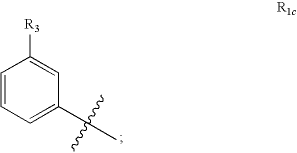

and

R₃ represents C₁₋₄alkyl, —NR₆R₆, —SO₂NR₆R₆, —SO₂NR₇COR₅, —NR₇COR₆ or Cy_{1c}, wherein the C₁₋₄alkyl group can be optionally substituted with one or more R₈ and Cy_{1c} can be optionally substituted with one or more R₉.

In another embodiment, the invention relates to the compounds of formula I wherein:

R₁ represents a ring of formula R_{1c}:

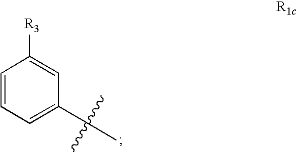

and

R₃ represents hydroxyC₁₋₄alkyl, Cy₂ₐC₁₋₄alkyl, —NR₆R₆, —SO₂NR₆R₆, —SO₂NR₇COR₆, —NR₇COR₆ or Cy_{1c}, wherein Cy_{1c} can be optionally substituted with one or more R₉ and Cy₂ₐ can be optionally substituted with one or more R₁₁.

In another embodiment, the invention relates to the compounds of formula I wherein:

R₁ represents a ring of formula R_{1c}:

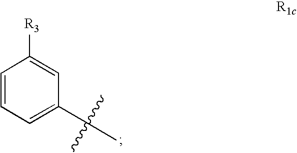

R₃ represents hydroxyC₁₋₄alkyl, Cy₂ₐC₁₋₄alkyl, —NR₆R₆, —SO₂NR₆R₆, —SO₂NR₇COR₆, —NR₇COR₆ or Cy_{1c}, wherein Cy_{1c} can be optionally substituted with one or more R₉ and Cy₂ₐ can be optionally substituted with one or more R₁₁;

R₅ represents C₁₋₄alkyl, hydroxyC₁₋₄alkyl or C₁₋₄alkoxyC₁₋₄alkyl; and

R₆ represents hydrogen or R₅.

In another embodiment, the invention relates to the compounds of formula I wherein:

R₁ represents a ring of formula R_{1c}:

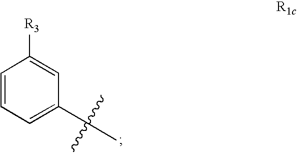

and

R₃ represents —SO₂NR₆R₆, —NR₇COR₆ or Cy₂ₐC₁₋₄alkyl, wherein Cy₂ₐ can be optionally substituted with one or more R₁₁.

In another embodiment, the invention relates to the compounds of formula I wherein:

R₁ represents a ring of formula R_{1c}:

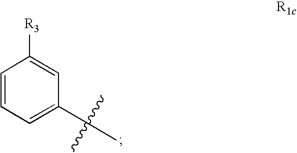

R₃ represents —SO₂NR₆R₆, —NR₇COR₆ or Cy₂ₐC₁₋₄alkyl, wherein Cy₂ₐ can be optionally substituted with one or more R₁₁; and

R₆ represents hydrogen or C₁₋₄alkyl optionally substituted with one or more R₁₀.

In another embodiment, the invention relates to the compounds of formula I wherein:

R₁ represents a ring of formula R_{1c}:

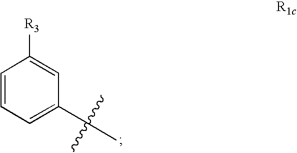

R₃ represents —SO₂NR₆R₆, —NR₇COR₆ or Cy₂ₐC₁₋₄alkyl, wherein Cy₂ₐ can be optionally substituted with one or more R₁₁; and

R₆ represents hydrogen, C₁₋₄alkyl, hydroxyC₁₋₄alkyl or C₁₋₄alkoxyC₁₋₄alkyl.

In another embodiment, the invention relates to the compounds of formula I wherein:

R₁ represents a ring of formula R_{1c}:

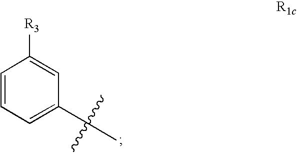

R₃ represents —SO₂NR₆R₆, —NR₇COR₆ or Cy₂ₐC₁₋₄alkyl, wherein Cy₂ₐ can be optionally substituted with one or more R₁₁; and

R₆ represents hydrogen or C₁₋₄alkyl.

In another embodiment, the invention relates to the compounds of formula I wherein:

R₁ represents a ring of formula R_{1d}:

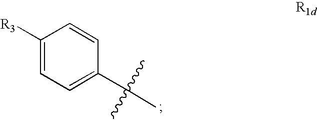

and

R₃ represents C₁₋₄alkyl, —NR₆R₆, —SO₂NR₆R₆ or Cy_{1c}, wherein the C₁₋₄alkyl group can be optionally subtituted with one or more R₈ and Cy_{1c} can be optionally substituted with one or more R₉.

In another embodiment, the invention relates to the compounds of formula I wherein:

R₁ represents a ring of formula R_{1d}:

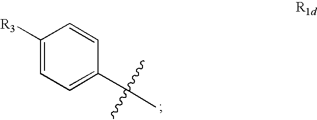

and

R₃ represents hydroxyC₁₋₄alkyl, Cy₂ₐC₁₋₄alkyl, —NR₆R₆, —SO₂NR₆R₆ or Cy_{1c}, wherein Cy_{1c} can be optionally substituted with one or more R₉ and wherein Cy₂ₐ can be optionally substituted with one or more R₁₁.

In another embodiment, the invention relates to the compounds of formula I wherein:

R₁ represents a ring of formula R_{1d}:

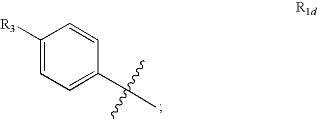

R₃ represents hydroxyC₁₋₄alkyl, Cy₂ₐC₁₋₄alkyl, —NR₆R₆, —SO₂NR₆R₆ or Cy_{1c},

wherein Cy_{1c} can be optionally substituted with one or more R₉ and wherein Cy₂ₐ can be optionally substituted with one or more R₁₁; and

R₆ represents hydrogen or C₁₋₄alkyl optionally substituted with one or more R₁₀.

In another embodiment, the invention relates to the compounds of formula I wherein:

R₁ represents a ring of formula R_{1d}:

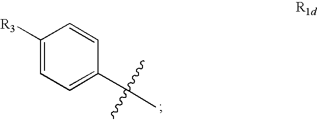

R₃ represents hydroxyC₁₋₄alkyl, Cy₂ₐC₁₋₄alkyl, —NR₆R₆, —SO₂NR₆R₆ or Cy_{1c},

wherein Cy_{1c} can be optionally substituted with one or more R₉ and wherein Cy₂ₐ can be optionally substituted with one or more R₁₁; and

R₆ represents hydrogen, C₁₋₄alkyl, hydroxyC₁₋₄alkyl or C₁₋₄alkoxyC₁₋₄alkyl.

In another embodiment, the invention relates to the compounds of formula I wherein:

R₁ represents a ring of formula R_{1d}:

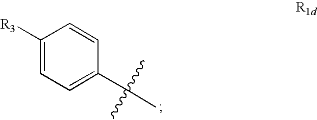

R₃ represents hydroxyC₁₋₄alkyl, Cy₂ₐC₁₋₄alkyl, —NR₆R₆, —SO₂NR₆R₆ or a ring of formula (i)-(iii), wherein Cy₂ₐ can be optionally substituted with one or more R₁₁;

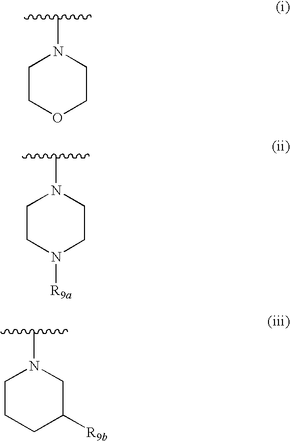

R₆ represents hydrogen, C₁₋₄alkyl, hydroxyC₁₋₄alkyl or C₁₋₄alkoxyC₁₋₄alkyl;

R₉ₐ represents hydrogen or C₁₋₄alkyl; and

R_{9b} represents hydrogen, C₁₋₄alkyl or hydroxy.

In another embodiment, the invention relates to the compounds of formula I wherein:

R₁ represents a ring of formula R_{1d}:

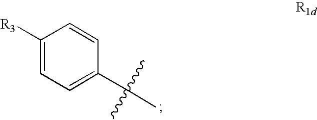

and

R₃ represents —SO₂NR₆R₆ or Cy_{1c} optionally substituted with one or more R₉.

In another embodiment, the invention relates to the compounds of formula I wherein:

R₁ represents a ring of formula R_{1d}:

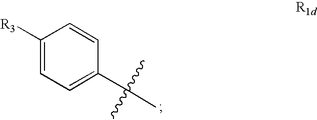

R₃ represents —SO₂NR₆R₆ or Cy_{1c} optionally substituted with one or more R₉; and

R₆ represents hydrogen or C₁₋₄alkyl optionally substituted with one or more R₁₀.

In another embodiment, the invention relates to the compounds of formula I wherein:

R₁ represents a ring of formula R_{1d}:

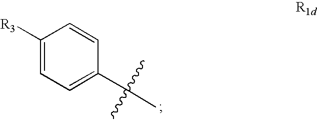

and

R₃ represents Cy_{1c} optionally substituted with one or more R₉.

In another embodiment, the invention relates to the compounds of formula I wherein:

R₁ represents a ring of formula R_{1d}:

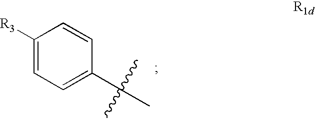

R₃ represents a ring of formula (i)-(iii)

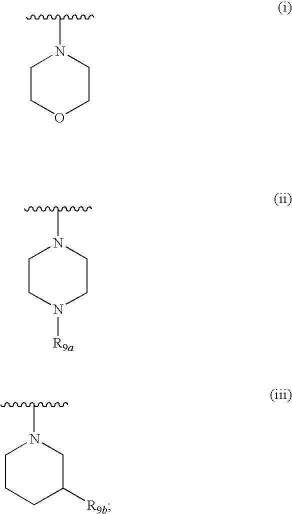

R₉ₐ represents hydrogen or C₁₋₄alkyl; and

R_{9b} represents hydrogen, C₁₋₄alkyl or hydroxy.

In another embodiment, the invention relates to the compounds of formula I wherein:

R₁ represents a ring of formula R₁ₑ:

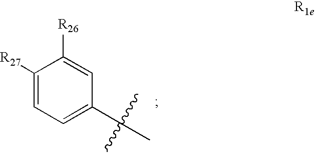

R₂₆ represents halogen or —SO₂NR₆R₆; and

R₂₇ represents C₁₋₄alkyl, C₁₋₄alkoxyalkyl or —OR₆.

In another embodiment, the invention relates to the compounds of formula I wherein:

R₁ represents a ring of formula R₁ₑ:

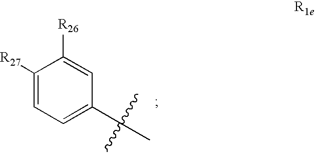

R₂₆ represents halogen or —SO₂NR₆R₆;

R₂₇ represents C₁₋₄alkyl, C₁₋₄alkoxyC₁₋₄alkyl or —OR₆; and

R₆ represents hydrogen, C₁₋₄alkyl, hydroxyC₁₋₄alkyl or C₁₋₄alkoxyC₁₋₄alkyl.

In another embodiment, the invention relates to the compounds of formula I wherein:

R₁ represents a ring of formula R₁ₑ:

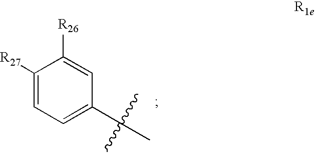

R₂₆ represents halogen or —SO₂NR₆R₆;

R₂₇ represents C₁₋₄alkyl C₁₋₄alkoxyC₁₋₄alkyl or —OR₆; and

R₆ represents hydrogen or C₁₋₄alkyl.

In another embodiment, the invention relates to the compounds of formula I wherein:

R₁ represents a group selected from R_{1c} and R_{1d}:

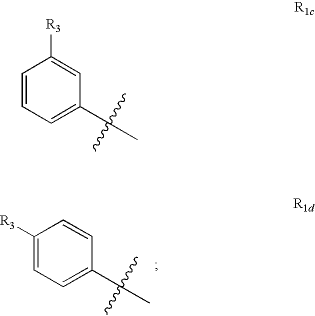

R₃ in R_{1c} represents —SO₂NR₆R₆, —NR₇COR₆ or Cy₂ₐC₁₋₄alkyl, wherein Cy₂ₐ can be optionally substituted with one or more R₁₁; and

R₃ in R_{1d} represents —SO₂NR₆R₆ or Cy_{1c} optionally substituted with one or more R₉.

In another embodiment, the invention relates to the compounds of formula I wherein:

R₁ represents a group selected from R_{1c} and R_{1d}:

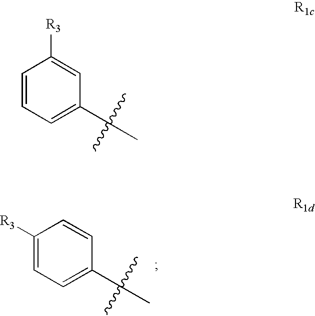

R₃ in R_{1c} represents —SO₂NR₆R₆, —NR₇COR₆ or Cy₂ₐC₁₋₄alkyl, wherein Cy₂ₐ can be optionally substituted with one or more R₁₁;

R₃ in R_{1d} represents —SO₂NR₆R₆ or Cy_{1c} optionally substituted with one or more R₉; and

R₆ represents hydrogen or C₁₋₄alkyl optionally substituted with one or more R₁₀.

In another embodiment, the invention relates to the compounds of formula I wherein R₂ represents phenyl or a 5- or 6-membered aromatic heterocycle bonded to the purine ring through a C atom, which can be optionally fused to a 5- or 6-membered saturated, partially unsaturated or aromatic carbocyclic or heterocyclic ring, wherein R₂ can contain from 1 to 4 heteroatoms selected from N, O and S, wherein the adjacent atoms to the C atom at the position of attachment to the purine ring are C atoms, wherein one or more C or S atoms of the 5- or 6-membered fused ring can be optionally oxidized forming CO, SO or SO₂ groups, and wherein R₂ can be optionally substituted with one or more R₄.

In another embodiment, the invention relates to the compounds of formula I wherein R₂ represents phenyl, pyridyl, indolyl or thienyl, which can all be optionally substituted with one or more R₄.

In another embodiment, the invention relates to the compounds of formula I wherein R₂ represents phenyl, 3-pyridyl, 5-indolyl or 3-thienyl which can all be optionally substituted with one or more R₄.

In another embodiment, the invention relates to the compounds of formula I wherein R₂ represents phenyl optionally substituted with one or more R₄.

In another embodiment, the invention relates to the compounds of formula I wherein R₂ represents phenyl substituted with one or more R₄.

In another embodiment, the invention relates to the compounds of formula I wherein R₂ represents a 5- or 6-membered aromatic heterocycle bonded to the purine ring through a C atom, which can be optionally fused to a 5- or 6-membered saturated, partially unsaturated or aromatic carbocyclic or heterocyclic ring, wherein R₂ contains from 1 to 4 heteroatoms selected from N, O and S, wherein one or more C or S atoms of the 5- or 6-membered fused ring can be optionally oxidized forming CO, SO or SO₂ groups, and wherein R₂ can be optionally substituted with one or more R₄.

In another embodiment, the invention relates to the compounds of formula I wherein R₂ represents a 5- or 6-membered aromatic heterocycle bonded to the purine ring through a C atom, which can be optionally fused to a 5- or 6-membered saturated, partially unsaturated or aromatic carbocyclic or heterocyclic ring, wherein R₂ contains from 1 to 4 heteroatoms selected from N, O and S, wherein the adjacent atoms to the C atom at the position of attachment to the purine ring are C atoms, wherein one or more C or S atoms of the 5- or 6-membered fused ring can be optionally oxidized forming CO, SO or SO₂ groups, and wherein R₂ can be optionally substituted with one or more R₄.

In another embodiment, the invention relates to the compounds of formula I wherein R₂ represents a 5- or 6-membered aromatic heterocycle bonded to the purine ring through a C atom, which can be optionally fused to a 5- or 6-membered aromatic carbocyclic or heterocyclic ring, wherein R₂ contains from 1 to 4 heteroatoms selected from N, O and S, wherein the adjacent atoms to the C atom at the position of attachment to the purine ring are C atoms, and wherein R₂ can be optionally substituted with one or more R₄.

In another embodiment, the invention relates to the compounds of formula I wherein R₂ represents a 5- or 6-membered aromatic heterocycle bonded to the purine ring through a C atom, wherein R₂ contains 1 or 2 heteroatoms selected from N, O and S, and wherein R₂ can be optionally substituted with one or more R₄.

In another embodiment, the invention relates to the compounds of formula I wherein R₂ represents a 5- or 6-membered aromatic heterocycle bonded to the purine ring through a C atom, wherein R₂ contains 1 or 2 heteroatoms selected from N, O and S, wherein the adjacent atoms to the C atom at the position of attachment to the purine ring are C atoms, and wherein R₂ can be optionally substituted with one or more R₄.

In another embodiment, the invention relates to the compounds of formula I wherein R₂ represents 3-pyridyl, 5-indolyl, 3-pyrrolyl, 3-thienyl or 4-pyrazolyl, which can be optionally substituted with one or more R₄.

In another embodiment, the invention relates to the compounds of formula wherein R₂ represents 3-pyridyl optionally substituted with one or more R₄.

In another embodiment, the invention relates to the compounds of formula wherein R₂ represents 4-pyrazolyl optionally substituted with one or more R₄.

In another embodiment, the invention relates to the compounds of formula wherein R₂ represents 3-thienyl optionally substituted with one or more R₄.

In another embodiment, the invention relates to the compounds of formula wherein R₂ represents 5-indolyl optionally substituted with one or more R₄.

In another embodiment, the invention relates to the compounds of formula wherein R₂ represents 3-pyrrolyl optionally substituted with one or more R₄.

In another embodiment, the invention relates to the compounds of formula wherein R₂ is optionally substituted with one or two R₄.

In another embodiment, the invention relates to the compounds of formula wherein R₂ represents 3-pyridyl substituted with one or two R₄.

In another embodiment, the invention relates to the compounds of formula wherein R₂ represents 4-pyrazolyl substituted with one or two R₄.

In another embodiment, the invention relates to the compounds of formula wherein R₂ represents 3-thienyl substituted with one or two R₄.

In another embodiment, the invention relates to the compounds of formula wherein R₂ represents 5-indolyl substituted with one or two R₄.

In another embodiment, the invention relates to the compounds of formula wherein R₂ represents 3-pyrrolyl substituted with one or two R₄.

In another embodiment, the invention relates to the compounds of formula wherein each R₄ independently represents C₁₋₄alkyl, halogen, —CN, —COR₆, —CO₂R₆, —CONR₆R₆, —OR₆, —SR₆, —SO₂R₅, —SO₂NR₆R₆, —SO₂NR₇COR₅, —NR₆R₆, —NR₇COR₆, —NR₇CONR₆R₆, —NR₇SO₂R₅ or Cy₁, wherein the C₁₋₄alkyl group can be optionally substituted with one or more R₈ and Cy₁ can be optionally substituted with one or more R₉.

In another embodiment, the invention relates to the compounds of formula I wherein each R₄ independently represents C₁₋₄alkyl, halogen, —CN, —CONR₆R₆, —OR₆, —SR₆, —SO₂R₆, —SO₂NR₆R₆, —NR₆R₆, —NR₇COR₆ or Cy₁, wherein Cy₁ can be optionally substituted with one or more R₉.

In another embodiment, the invention relates to the compounds of formula I wherein Cy₁ in R₄ is Cy_{1b} and Cy_{1b} represents a 3- to 7-membered saturated monocyclic heterocycle which contains 1 or 2 heteroatoms selected from N, S and O, wherein said ring can be bonded to the rest of the molecule through any available C or N atom, and wherein one or more C or S ring atoms can be optionally oxidized forming CO, SO or SO₂ groups, wherein said Cy_{1b} can be optionally substituted with one or more R₉.

In another embodiment, the invention relates to the compounds of formula I wherein Cy₁ in R₄ is Cy_{1d} and Cy_{1d} represents a 3- to 7-membered saturated monocyclic heterocycle which contains 1 or 2 heteroatoms selected from N, S and O with the proviso that at least it contains 1 N atom, wherein said ring is bonded to the rest of the molecule through a N atom, and wherein one or more C or S ring atoms can be optionally oxidized forming CO, SO or SO₂ groups, wherein said Cy_{1d} can be optionally substituted with one or more R₉.

In another embodiment, the invention relates to the compounds of formula I wherein Cy₁ in R₄ is Cy_{1c} and Cy_{1c} represents a 5- or 6-membered saturated monocyclic heterocycle which contains 1 or 2 heteroatoms selected from N, S and O with the proviso that it contains at least 1 N atom, wherein said ring is bonded to the rest of the molecule through a N atom, wherein one or more C or S ring atoms can be optionally oxidized forming CO, SO or SO₂ groups, and wherein said Cy_{1c} can be optionally substituted with one or more R₉.

In another embodiment, the invention relates to the compounds of formula I wherein:

each R₄ independently represents C₁₋₄alkyl, halogen, —CN, —CONR₆R₆, —OR₆, —SR₆, —SO₂R₆, —SO₂NR₆R₆, —NR₆R₆, —NR₇COR₆ or Cy_{1b}, wherein Cy_{1b} can be optionally substituted with one or more R₉.

In another embodiment, the invention relates to the compounds of formula I wherein each R₄ independently represents C₁₋₄alkyl, halogen, —CONR₆R₆, —SR₆, —SOR₆, —SO₂R₆, —NR₆R₆, —NR₇SO₂R₅, —NR₇CONR₆R₆ or Cy_{1d}, wherein the C₁₋₄alkyl group can be optionally substituted with one or more R₈ and Cy_{1d} can be optionally substituted with one or more R₉.

In another embodiment, the invention relates to the compounds of formula I wherein each R₄ independently represents C₁₋₄alkyl, halogen, hydroxyC₁₋₄alkyl, C₁₋₄alkoxyC₁₋₄alkyl, —CONR₆R₆, —SR₆, —SOR₅, —SO₂R₅, —NR₆R₆, —NR₇SO₂R₅, —NR₇CONR₆R₆ or Cy_{1c}, wherein Cy_{1c} can be optionally substituted with one or more R₉.

In another embodiment, the invention relates to the compounds of formula I wherein R₆ in R₄ represents hydrogen or R₅ and R₅ represents C₁₋₄alkyl optionally substituted with one or more R₁₀.

In another embodiment, the invention relates to the compounds of formula I wherein R₆ in R₄ represents hydrogen or R₅ and R₅ represents C₁₋₄alkyl, hydroxyC₁₋₄alkyl or C₁₋₄alkoxyC₁₋₄alkyl.

In another embodiment, the invention relates to the compounds of formula I wherein:

R₂ represents phenyl, pyridyl, indolyl or thienyl which can be optionally substituted with one or more R₄; and

R₄ represents C₁₋₄alkyl, halogen, —CN, —CONR₆R₆, —OR₆, —SR₆, —SO₂R₅, —SO₂NR₆R₆, —NR₆R₆, —NR₇COR₆ or Cy_{1b}, wherein Cy_{1b} can be optionally substituted with one or more R₉.

In another embodiment, the invention relates to the compounds of formula I wherein:

R₂ represents phenyl, pyridyl, indolyl or thienyl which can be optionally substituted with one or more R₄; and

R₄ represents C₁₋₄alkyl, halogen, —CN, —CONR₆R₆, —OR₆, —SR₆, —SO₂R₅, —SO₂NR₆R₆, —NR₆R₆ or —NR₇COR₆.

In another embodiment, the invention relates to the compounds of formula I wherein:

R₂ represents a group of formula R₂ₐ:

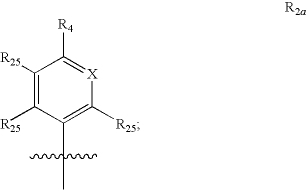

R₄ represents —OR₆, —NR₆R₆ or Cy_{1b}, wherein Cy_{1b} can be optionally substituted with one or more R₉;

X represents CR₂₅ or N; and

each R₂₅ independently represents hydrogen, halogen, C₁₋₄alkyl, C₁₋₄alkoxy, haloC₁₋₄alkoxy or —SC₁₋₄alkyl.

In another embodiment, the invention relates to the compounds of formula I wherein:

R₂ represents a group of formula R₂ₐ:

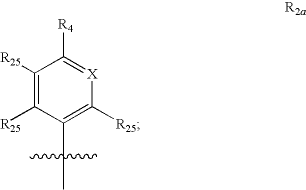

R₄ represents —OR₆, —NR₆R₆ or Cy_{1b}, wherein Cy_{1b} can be optionally substituted with one or more R₉;

X represents N; and

each R₂₅ independently represents hydrogen, halogen, C₁₋₄alkyl, C₁₋₄alkoxy, haloC₁₋₄alkoxy or —SC₁₋₄alkyl.

In another embodiment, the invention relates to the compounds of formula I wherein:

R₂ represents a group of formula:

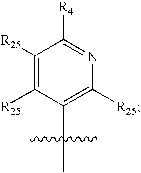

and

each R₂₅ independently represents hydrogen, halogen or C₁₋₄alkyl.

In another embodiment, the invention relates to the compounds of formula I wherein:

R₂ represents a group of formula:

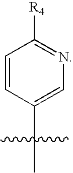

In another embodiment, the invention relates to the compounds of formula I wherein:

R₂ represents a group of formula:

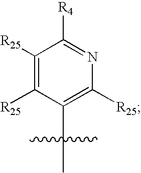

R₄ represents —NR₆R₆ or Cy_{1d}, wherein Cy_{1d} can be optionally substituted with one or more R₉; and

each R₂₅ independently represents hydrogen, halogen or C₁₋₄alkyl.

In another embodiment, the invention relates to the compounds of formula I wherein:

R₂ represents a group of formula:

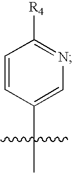

and

R₄ represents —NR₆R₆ or Cy_{1d}, wherein Cy_{1d} can be optionally substituted with one or more R₉.

In another embodiment, the invention relates to the compounds of formula I wherein:

R₂ represents a group of formula:

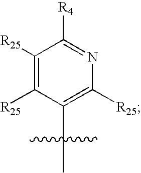

R₄ represents —NR₆R₆ or Cy_{1c}, wherein Cy_{1c} can be optionally substituted with one or more R₉; and

each R₂₅ independently represents hydrogen, halogen or C₁₋₄alkyl.

In another embodiment, the invention relates to the compounds of formula I wherein:

R₂ represents a group of formula:

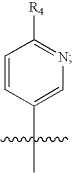

and

R₄ represents —NR₆R₆ or Cy_{1c}, wherein Cy_{1c} can be optionally substituted with one or more R₉.

In another embodiment, the invention relates to the compounds of formula I wherein:

R₂ represents a group of formula:

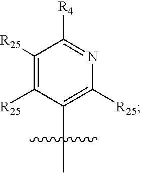

R₄ represents —NR₆R₆ or Cy_{1c}, wherein Cy_{1c} can be optionally substituted with one or more R₉;

R₆ represents C₁₋₄alkyl optionally substituted with one or more R₁₀; and

each R₂₅ independently represents hydrogen, halogen or C₁₋₄alkyl.

In another embodiment, the invention relates to the compounds of formula I wherein:

R₂ represents a group of formula:

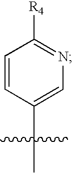

R₄ represents —NR₆R₆ or Cy_{1c}, wherein Cy_{1c} can be optionally substituted with one or more R₉; and

R₆ represents C₁₋₄alkyl optionally substituted with one or more R₁₀.

In another embodiment, the invention relates to the compounds of formula I wherein:

R₂ represents a group of formula:

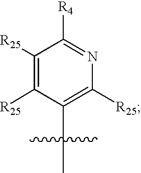

R₄ represents —NR₆R₆ or Cy_{1c}, wherein Cy_{1c} can be optionally substituted with one or more R₉;

R₆ represents C₁₋₄alkyl, hydroxyC₁₋₄alkyl or C₁₋₄alkoxyC₁₋₄alkyl;

R₉ represents C₁₋₄alkyl, —OR₁₈, —CONR₁₈R₁₈ or —COR₁₈; and

each R₂₅ independently represents hydrogen, halogen or C₁₋₄alkyl.

In another embodiment, the invention relates to the compounds of formula I wherein:

R₂ represents a group of formula:

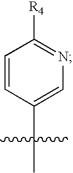

R₄ represents —NR₆R₆ or Cy_{1c}, wherein Cy_{1c} can be optionally substituted with one or more R₉;

R₆ represents C₁₋₄alkyl, hydroxyC₁₋₄alkyl or C₁₋₄alkoxyC₁₋₄alkyl; and

R₉ represents C₁₋₄alkyl, —OR₁₈, —CONR₁₈R₁₈ or —COR₁₈.

In another embodiment, the invention relates to the compounds of formula I wherein:

R₂ represents a group of formula:

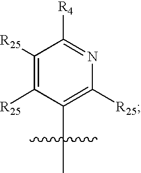

R₄ represents —NR₆R₆;

R₆ represents C₁₋₄alkyl optionally substituted with one or more R₁₀; and

each R₂₅ independently represents hydrogen, halogen or C₁₋₄alkyl.

In another embodiment, the invention relates to the compounds of formula I wherein:

R₂ represents a group of formula:

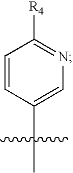

R₄ represents —NR₆R₆; and

R₆ represents C₁₋₄alkyl optionally substituted with one or more R₁₀.

In another embodiment, the invention relates to the compounds of formula I wherein:

R₂ represents a group of formula:

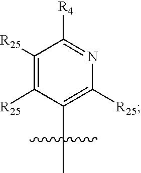

R₄ represents —NR₆R₆;

R₆ represents C₁₋₄alkyl, hydroxyC₁₋₄alkyl or C₁₋₄alkoxyC₁₋₄alkyl; and

each R₂₅ independently represents hydrogen, halogen or C₁₋₄alkyl.

In another embodiment, the invention relates to the compounds of formula I wherein:

R₂ represents a group of formula:

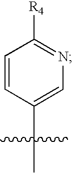

R₄ represents —NR₆R₆; and

R₆ represents C₁₋₄alkyl, hydroxyC₁₋₄alkyl or C₁₋₄alkoxyC₁₋₄alkyl.

In another embodiment, the invention relates to the compounds of formula I wherein R₂ represents a group of formula:

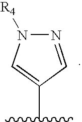

In another embodiment, the invention relates to the compounds of formula I wherein:

R₂ represents a group of formula:

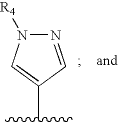

R₄ represents C₁₋₄alkyl optionally substituted with one or more R₈.

In another embodiment, the invention relates to the compounds of formula I wherein:

R₂ represents a group of formula:

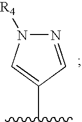

and

R₄ represents C₁₋₄alkyl, hydroxyC₁₋₄alkyl or C₁₋₄alkoxyC₁₋₄alkyl.

In another embodiment, the invention relates to the compounds of formula I wherein R₂ represents

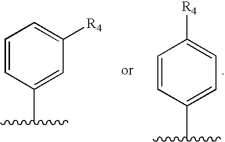

In another embodiment, the invention relates to the compounds of formula I wherein R₂ represents a group of formula:

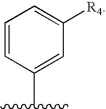

In another embodiment, the invention relates to the compounds of formula I wherein:

R₂ represents a group of formula:

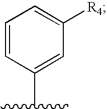

and

R₄ represents —CONR₆R₆, —SR₆, —SOR₅, or —SO₂R₅.

In another embodiment, the invention relates to the compounds of formula I wherein:

R₂ represents a group of formula:

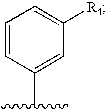

R₄ represents —CONR₆R₆, —SR₆, —SOR₅, or —SO₂R₅;

R₅ represents C₁₋₄alkyl optionally substituted with one or more R₁₀; and

R₆ represents hydrogen or R₅.

In another embodiment, the invention relates to the compounds of formula I wherein:

R₂ represents a group of formula:

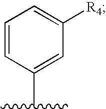

R₄ represents —CONR₆R₆, —SR₆, —SOR₅, or —SO₂R₅;

R₅ represents C₁₋₄alkyl, haloC₁₋₄alkyl, hydroxyC₁₋₄alkyl or C₁₋₄alkoxyC₁₋₄alkyl; and

R₆ represents hydrogen or R₅.

In another embodiment, the invention relates to the compounds of formula I wherein R₂ represents a group of formula:

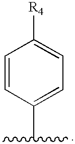

In another embodiment, the invention relates to the compounds of formula I wherein:

R₂ represents a group of formula:

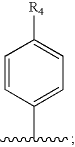

and

R₄ represents —NR₆R₆, —NR₇SO₂R₅, or —NR₇CONR₆R₆.

In another embodiment, the invention relates to the compounds of formula I wherein:

R₂ represents a group of formula:

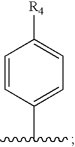

R₄ represents —NR₆R₆, —NR₇SO₂R₅, or —NR₇CONR₆R₆;

R₅ represents C₁₋₄alkyl optionally substituted with one or more R₁₀; and

R₆ represents hydrogen or R₅.

In another embodiment, the invention relates to the compounds of formula I wherein:

R₂ represents a group of formula:

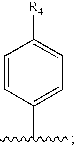

R₄ represents —NR₆R₆, —NR₇SO₂R₅, or —NR₇CONR₆R₆;

R₅ represents C₁₋₄alkyl, hydroxyC₁₋₄alkyl or C₁₋₄alkoxyC₁₋₄alkyl; and

R₆ represents hydrogen or R₅.

Furthermore, the present invention covers all possible combinations of the particular and preferred embodiments described above.

In another embodiment, the invention relates to a compound of formula I which provides more than 50% inhibition of JAK3 activity at 10 μM, more preferably at 1 μM and still more preferably at 0.1 μM, in a JAK3 assay such as the one described in example 27.

In another embodiment, the invention relates to a compound of formula I selected from the list of compounds described in examples 1 to 26a.

The compounds of the present invention contain one or more basic nitrogens and may, therefore, form salts with organic or inorganic acids. Examples of these salts include: salts with inorganic acids such as hydrochloric acid, hydrobromic acid, hydroiodic acid, nitric acid, perchloric acid, sulfuric acid or phosphoric acid; and salts with organic acids such as methanesulfonic acid, trifluoromethanesulfonic acid, ethanesulfonic acid, benzenesulfonic acid, p-toluenesulfonic acid, fumaric acid, oxalic acid, acetic acid, maleic acid, ascorbic acid, citric acid, lactic acid, tartaric acid, malonic acid, glycolic acid, succinic acid and propionic acid, among others. Some of the compounds of the present invention may contain one or more acidic protons and, therefore, they may also form salts with bases. Examples of these salts include: salts with inorganic cations such as sodium, potassium, calcium, magnesium, lithium, aluminium, zinc, etc; and salts formed with pharmaceutically acceptable amines such as ammonia, alkylamines, hydroxylalkylamines, lysine, arginine, N-methylglucamine, procaine and the like.

There is no limitation on the type of salt that can be used, provided that these are pharmaceutically acceptable when used for therapeutic purposes. The term pharmaceutically acceptable salt refers to those salts which are, according to medical judgment, suitable for use in contact with the tissues of humans and other mammals without undue toxicity, irritation, allergic response and the like. Pharmaceutically acceptable salts are well known in the art.

The salts of a compound of formula I can be obtained during the final isolation and purification of the compounds of the invention or can be prepared by treating a compound of formula I with a sufficient amount of the desired acid or base to give the salt in a conventional manner. The salts of the compounds of formula I can be converted into other salts of the compounds of formula I by ion exchange using ionic exchange resins.

The compounds of formula I and their salts may differ in some physical properties but they are equivalent for the purposes of the present invention. All salts of the compounds of formula I are included within the scope of the invention.

The compounds of the present invention may form complexes with solvents in which they are reacted or from which they are precipitated or crystallized. These complexes are known as solvates. As used herein, the term solvate refers to a complex of variable stoichiometry formed by a solute (a compound of formula I or a salt thereof) and a solvent. Examples of solvents include pharmaceutically acceptable solvents such as water, ethanol and the like. A complex with water is known as a hydrate. Solvates of compounds of the invention (or salts thereof), including hydrates, are included within the scope of the invention.

The compounds of formula I may exist in different physical forms, i.e. amorphous and crystalline forms. Moreover, the compounds of the invention may have the ability to crystallize in more than one form, a characteristic which is known as polymorphism. Polymorphs can be distinguished by various physical properties well known in the art such as X-ray diffraction pattern, melting point or solubility. All physical forms of the compounds of formula I, including all polymorphic forms (“polymorphs”) thereof, are included within the scope of the invention.

Some of the compounds of the present invention may exist as several diastereoisomers and/or several optical isomers. Diastereoisomers can be separated by conventional techniques such as chromatography or fractional crystallization. Optical isomers can be resolved by conventional techniques of optical resolution to give optically pure isomers. This resolution can be carried out on any chiral synthetic intermediate or on products of formula I. Optically pure isomers can also be individually obtained using enantiospecific synthesis. The present invention covers all individual isomers as well as mixtures thereof (for example racemic mixtures or mixtures of diastereomers), whether obtained by synthesis or by physically mixing them.

The compounds of formula I can be obtained by following the processes described below. As it will be obvious to one skilled in the art, the exact method used to prepare a given compound may vary depending on its chemical structure. Moreover, in some of the processes described below it may be necessary or advisable to protect the reactive or labile groups by conventional protecting groups. Both the nature of these protecting groups and the procedures for their introduction or removal are well known in the art (see for example Greene T. W. and Wuts P. G. M, “Protective Groups in Organic Synthesis”, John Wiley & Sons, 3^{rd} edition, 1999). As an example, as protecting groups of an amino function the tetrahydropyranyl (THP) group can be used. Whenever a protecting group is present, a later deprotection step will be required, which can be performed under standard conditions in organic synthesis, such as those described in the above-mentioned reference.

Unless otherwise stated, in the methods described below the meanings of the different substituents are the meanings described above with regard to a compound of formula I.

In general, compounds of formula I can be obtained in three steps by the method described in Scheme 1:

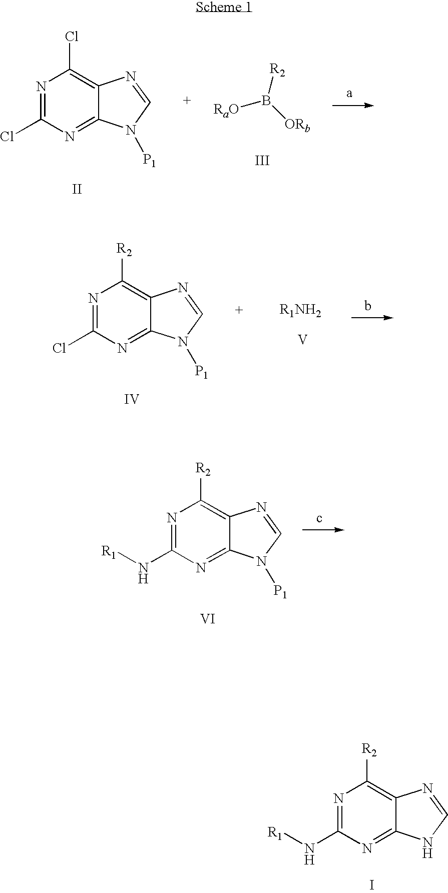

wherein R₁ and R₂ have the meaning previously described in relation with a compound of formula I; P₁ represents an amine protecting group, such as for example tetrahydropyranyl (THP); and Rₐ and R_{b} represent H or C₁₋₄alkyl, or can be bonded forming together with the B and O atoms a 5- or 6-membered ring that can be optionally substituted with one or more methyl groups.

In a first step (step a), a compound of formula II is reacted with a compound of formula III under the conditions reported in the literature for Suzuki couplings to give a compound of formula IV. For example, the reaction can be carried out in the presence of a base, such as Na₂CO₃, NaOH, Cs₂CO₃, CsF or Ba(OH)₂, and a palladium catalyst, such as Pd(PPh₃)₄, Pd₂(dba)₃ or Pd(OAc)₂, in a solvent, such as dimethoxyethane, toluene, N,N-dimethylformamide, tetrahydrofuran or dioxane, optionally in the presence of water, and heating, preferably at around 90° C.

In step b a compound of formula IV is reacted with an amine of formula V in the presence of a base, such as potassium tert-butoxide, Cs₂CO₃, LiHMDS, K₂CO₃ or K₂PO₃, in the presence of a phosphine, such as BINAP or 4,5-bis(diphenylphosphine)-9,9-dimethyl-9H-xanthene (Xantphos), and of a palladium catalyst, such as Pd₂(dba)₃ or Pd(OAc)₂, in a solvent such as toluene, dioxane or tetrahydrofuran, and heating, preferably at around 100° C., to give a compound of formula VI.

Finally, the protecting group of a compound of formula VI is cleavaged under the standard conditions described in the literature to give a compound I. For example, in case THP is used as P₁, the cleavage is performed by treating compound VI with a 4M dioxane/HCl_{(g)} mixture at room temperature.

Alternatively, the compounds of formula I can also be obtained using the method described in Scheme 2:

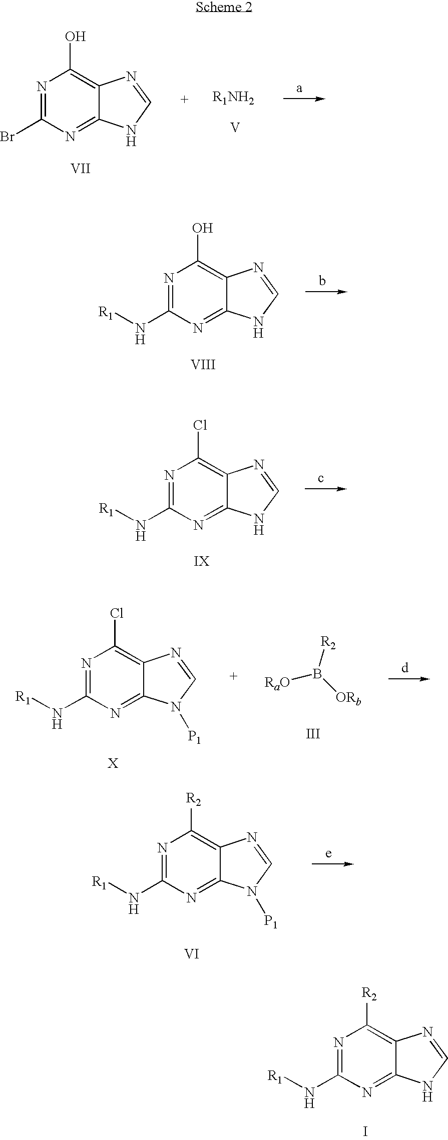

wherein R₁, R₂, P₁, Rₐ and R_{b} have the meaning previously described.

Step a is carried out by reacting VII with an amine of formula V in a solvent, such as 2-methoxyethanol or n-butanol, heating, preferably at around 120° C., to give a compound of formula VIII.

Thereafter a compound of formula VIII is converted into a compound of formula IX in the presence of a chlorinating agent, such as POCl₃ or dichlorophenylphosphoric acid, and a base such as N,N-dimethylaniline, and heating, preferably at reflux.

In a third step, the amino group of a compound of formula IX is protected with an amine protecting group P₁, such as THP, under standard conditions, to give a compound of formula X. If P₁ is THP, the reaction is carried out in the presence of an acid, such as p-toluensulfonic acid, pyridinium p-toluensulfonate, Amberlyst® or HCl, in a solvent, such as ethyl acetate, and heating, preferably at around 50° C.

The conversion of X into a compound of formula VI by reaction with a compound III is carried out in the same conditions described in step a of Scheme 1.

Finally, a compound of formula VI is deprotected following the method described in step c of Scheme 1, to give a compound of formula I.

Alternatively, a compound of formula I wherein R₂=6-R₄*-pyridin-3-yl and R₄*=—NR₆R₆ or Cy₁ bonded through a N atom to the pyridine ring (compounds Ia) can be also obtained by the method described in Scheme 3:

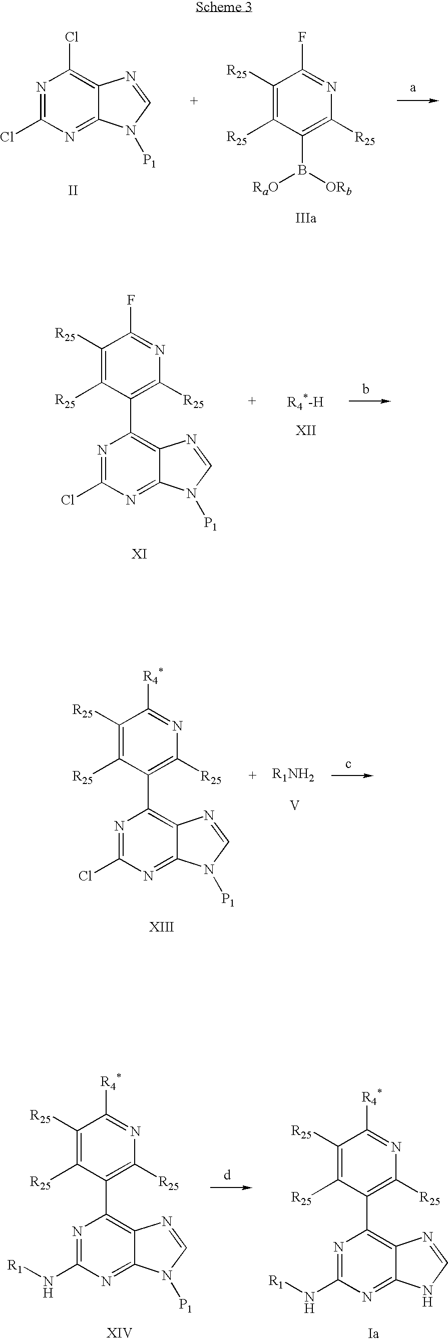

wherein R₄* represents —NR₆R₆ or Cy₁ bonded through a N atom to the pyridine ring, each R₂₅ independently represents hydrogen, halogen, C₁₋₄alkyl, C₁₋₄alkoxy, haloC₁₋₄alkoxy or —SC₁₋₄alky, and P₁, R₁, Cy₁, Rₐ, and R_{b} have the meaning previously described.

In a first step, the compound of formula II is allowed to react with a compound of formula IIIa following a similar procedure to that described for step a of Scheme 1 to give a compound of formula XI.

The compound of formula XI thus obtained is allowed to react with an amine of formula XII, in a solvent such as n-butanol, in the presence of a base such as diisopropylethylamine, and heating, preferably at around 120° C., to give a compound of formula XIII.

The compound of formula XIII thus obtained is then allowed to react with an amine of formula V following the procedure described in step b of Scheme 1 to give a compound of formula XIV.

Finally a compound of formula XIV is deprotected to give a compound of formula Ia following the procedure described in step c of Scheme 1.

Alternatively, a compound of formula Ia can be obtained from a compound of formula XI in three steps, as shown in Scheme 4:

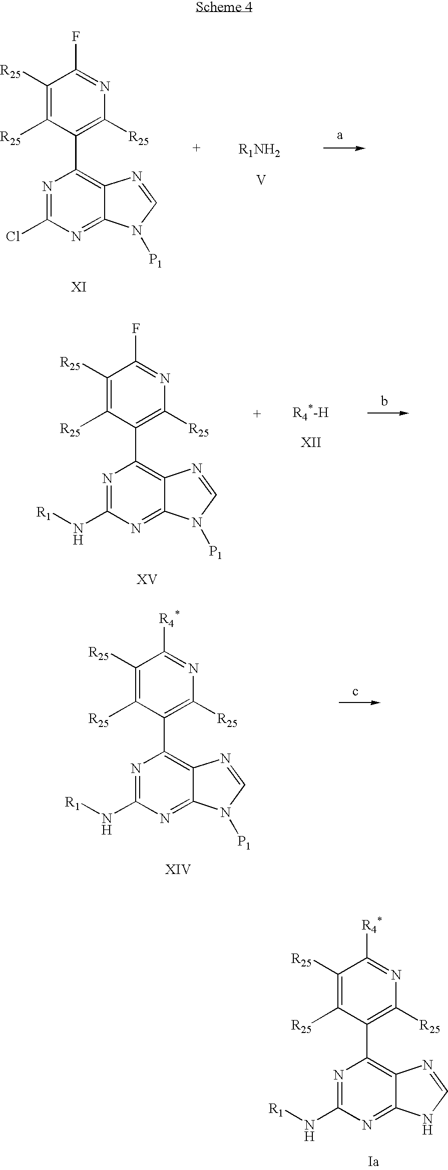

wherein P₁, R₁, R₄* and R₂₅ have the meaning previously described.

In a first step, a compound of formula XI is allowed to react with an amine of formula V following the procedure described in step b of Scheme 1 to yield a compound of formula XV.

Next, a compound of formula XV is allowed to react with an amine of formula XII following a similar procedure to that described in step b of Scheme 3, to give a compound of formula XIV.

And, finally, the amino protecting group of a compound of formula XIV is cleavaged using the method described in step c of Scheme 1, to give a compound of formula Ia.

The compounds of formula II can be prepared from 2,6-dichloropurine following any of the methods described in the literature for protecting amino groups.

The compounds of formula III and IIIa are commercially available or can be prepared by well-known methods described in the literature.

The compounds of formula III with a cyclic structure (IIIb) can be prepared from a compound of formula XVI following the procedure shown in Scheme 5:

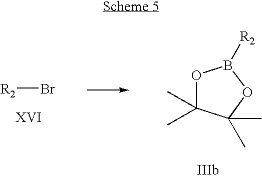

wherein R₂ has the meaning previously described.

The reaction is carried out by reacting a compound of formula XVI with bis(pinacolato)diboron and [1,1′-bis(diphenylphosphine)ferrocene]-dichloropalladium in the presence of a base, such as potassium acetate, in a solvent, such as N,N-dimethylformamide or dioxane, and heating, preferably at around 90° C., to give a compound of formula IIIb.

The compounds of formula V, VII, XII and XVI are commercially available or can be prepared by well-known methods described in the literature, and can be protected with suitable protecting groups.

Furthermore, some compounds of the present invention can also be obtained from other compounds of formula I by appropriate conversion reactions of functional groups in one or several steps, using well-known reactions in organic chemistry under the standard experimental conditions.

Said transformations can be carried out upon R₁ or R₂ groups and include, for example:

the reduction of a nitro group to give an amino group, for example by treatment with hydrogen, hydrazine or formic acid in the presence of a suitable catalyst such as Pd/C; or by treatment with sodium borohydride in the presence of NiCl₂, or SnCl₂;

the substitution of a primary or secondary amine by treatment with an alkylating agent under standard conditions, or by reductive amination, i.e. by treatment with an aldehyde or a ketone in the presence of a reducing agent such as sodium cyanoborohydride or sodium triacetoxyborohydride;

the conversion of an amine into a sulfonamide by reaction with a sulfonyl halide, such as sulfonyl chloride, optionally in the presence of catalytic amounts of a base such as 4-dimethylaminopyridine, in a suitable solvent such as dioxane, chloroform, dichloromethane or pyridine, optionally in the presence of a base such as triethylamine or pyridine;

the conversion of an amine into an amide, carbamate or urea under standard conditions;

the alkylation of an amide by treatment with an alkylating agent under basic conditions;

the conversion of an alcohol into an ether, ester or carbamate under standard conditions;

the alkylation of a thiol to give a thioeter under standard conditions;

the partial or total oxidation of an alcohol to give ketones, aldehydes or carboxylic acids under standard oxidizing conditions;

the reduction of an aldehyde or ketone by treatment with a reducing agent such as sodium borohydride;

the reduction of a carboxylic acid or a carboxylic acid derivative to an alcohol by treatment with a reducing agent such as diisobutylaluminium hydride or LiAlH₄;

the oxidation of a thioeter to a sulfoxide or sulfone under standard conditions;

the conversion of an alcohol into a halogen by reaction with SOCl₂, PBr₃, tetrabutylammonium bromide in the presence of P₂O₅, or PI₃;

the conversion of halogen into an amine by reaction with an amine, optionally in the presence of a suitable solvent, and preferably heating; and

the conversion of a primary amide into a —CN group under standard conditions.

Likewise, any of the aromatic rings of the compounds of the present invention can undergo electrophilic aromatic substitution reactions or nucleophilic aromatic substitution reactions, widely described in the literature.

Some of these interconversion reactions are explained in greater detail in the examples.

As it will be obvious to those skilled in the art, these interconversion reactions can be carried out upon the compounds of formula I as well as upon any suitable synthesis intermediate thereof.

As mentioned above, the compounds of the present invention act by inhibiting JAK/STAT signaling pathways, particularly by inhibiting JAK3 activity. Therefore, the compounds of the invention are expected to be useful to treat or prevent diseases in which JAKs, particularly JAK3, play a role in mammals, including human beings. These diseases include, but are not limited to, transplant rejection; immune, autoimmune or inflammatory diseases; neurodegenerative diseases; and proliferative disorders (see e.g. O'Shea J. J. et al, Nat. Rev. Drug. Discov. 2004, 3(7):555-64; Cetkovic-Cvrlje M. et al, Curr. Pharm. Des. 2004, 10(15):1767-84; Cetkovic-Cvrlje M. et al, Arch. Immunol. Ther. Exp. (Warsz), 2004, 52(2):69-82).

Acute or chronic transplant rejection reactions that can be prevented or treated with the compounds of the present invention include any kind of cell, tissue or organ xenotransplants or allografts, such as of heart, lung, liver, kidney, pancreas, uterus, joints, pancreatic islets, bone marrow, limbs, cornea, skin, hepatocytes, pancreatic beta cells, pluripotential cells, neuronal cells and myocardial cells, as well as graft-versus-host reactions (see e.g. Rousvoal G. et al, Transpl. Int. 2006, 19(12):1014-21; Borie D C. et al, Transplantation 2005, 79(7):791-801; Paniagua R. et al, Transplantation 2005, 80(9):1283-92; Higuchi T. et al, J. Heart Lung Transplant. 2005, 24(10):1557-64; Saemann M D. et al, Transpl Int. 2004, 17(9):481-89; Silva Jr H T. et al, Drugs 2006, 66(13):1665-1684).

Immune, autoimmune or inflammatory diseases that can be treated or prevented with the compounds of the present invention include among others, rheumatic diseases (e.g. rheumatoid arthritis and psoriatic arthritis), autoimmune hematological disorders (e.g. hemolytic anemia, aplastic anemia, idiopathic thrombocytopenia, and neutropenia), autoimmune gastritis and inflammatory bowel diseases (e.g. ulcerative colitis and Crohn's disease), scleroderma, type I diabetes and complications from diabetes, type B hepatitis, type C hepatitis, primary biliary cirrhosis, myasthenia gravis, multiple sclerosis, systemic lupus erythematosus, psoriasis, atopic dermatitis, contact dermatitis, eczema, skin sunburns, suppression of HIV replication, infertility of autoimmune origin, autoimmune thyroid disease (Grave's disease), interstitial cystitis, and mast cell-mediated allergic reactions such as asthma, angiodema, anaphylaxis, bronchitis, rhinitis and sinusitis (see e.g. Sorbera L A. et al, Drugs of the Future 2007, 32(8):674-680; O'Shea J. J. et al, Nat. Rev. Drug. Discov. 2004, 3(7):555-64; Cetkovic-Cvrlje M. et al, Curr. Pharm. Des. 2004, 10(15):1767-84; Muller-Ladner U. et al, J. Immunol. 2000, 164(7): 3894-3901; Walker J G. et al, Ann. Rheum. Dis. 2006, 65(2):149-56; Milici A J. et al, Arthritis Rheum 0.2006, 54 (9, Suppl): abstr 789; Kremer J M. et al, Arthritis Rheum. 2006, 54, 4116, presentation no. L40; Cetkovic-Cvrlje M. et al, Arch Immunol. Ther. Exp. (Warsz), 2004, 52(2):69-82; Malaviya R. et al, J. Pharmacol. Exp. Ther. 2000, 295(3):912-26; Malaviya R. et al, J. Biol. Chem. 1999, 274(38):27028-38; Wilkinson B et al, Ann. Rheum. Dis. 2007, 66(Suppl 2): Abst. THU0099; Matsumoto M. et al, J. Immunol. 1999, 162(2):1056-63).

Neurodegenerative diseases that can be treated or prevented with the compounds of the present invention include, among others, amyotrophic lateral sclerosis and Alzheimer's disease (see e.g. Trieu V N. et al, Biochem. Biophys. Res. Commun. 2000, 267(1):22-5).

Proliferative disorders that can be treated or prevented with the compounds of the present invention include, among others, leukemias, lymphomas, glioblastoma multiforme, colon carcinoma, as well as thromboembolic and allergic complications associated with these diseases (see e.g. Sudbeck E A. et al, Clin. Cancer Res. 1999, 5(6):1569-82; Narla R K. et al, Clin. Cancer Res. 1998, 4(10):2463-71; Lin Q. et al, Am J. Pathol. 2005, 167(4):969-80; Tibbles H E. et al, J. Biol. Chem. 2001, 276(21):17815-22).

Biological assays that can be used to determine the ability of a compound to inhibit JAKs, particularly JAK3, are well known in the art. For example, a compound to be tested can be incubated in the presence of JAK3 to determine whether inhibition of JAK3 enzymatic activity occurs, as described in the assay of example 27. Other in vitro useful assays that can be used to measure JAK3-inhibitory activity include cellular assays, for example IL-2-induced proliferation of human T lymphocytes. The immunosuppressive activity of the compounds of the invention can be tested using standard in vivo animal models for immune and autoimmune diseases, which are well known in the art. For example, the following assays can be used: delayed-type hypersensitivity (DTH) (see e.g. the method disclosed in Kudlacz E. et al, Am J. Transplant. 2004, 4(1):51-7, the contents of which are incorporated herein by reference), rheumatoid arthritis models such as collagen-induced arthritis (see e.g. the method disclosed in Holmdahl R et al, APMIS, 1989, 97(7):575-84, the contents of which are incorporated herein by reference), multiple sclerosis models such as experimental autoimmune encephalomyelitis (EAE) (see e.g. the method disclosed in González-Rey et al, Am. J. Pathol. 2006, 168(4): 1179-88, the contents of which are incorporated herein by reference) and transplant rejection models (see e.g. the various animal models disclosed in the references listed above in relation to the treatment or prevention of transplant rejection, incorporated herein by reference).

For selecting active compounds, testing at 10 μM must result in an activity of more than 50% inhibition of JAK3 activity in the test provided in example 27. More preferably, when tested in this assay compounds should exhibit more than 50% inhibition at 1 μM, and still more preferably, they should exhibit more than 50% inhibition at 0.1 μM.

The present invention also relates to a pharmaceutical composition that comprises a compound of the present invention (or a pharmaceutically acceptable salt or solvate thereof) and one or more pharmaceutically acceptable excipients. The excipients must be “acceptable” in the sense of being compatible with the other ingredients of the composition and not deleterious to the recipients thereof.

The compounds of the present invention can be administered in the form of any pharmaceutical formulation, the nature of which, as it is well known, will depend upon the nature of the active compound and its route of administration. Any route of administration may be used, for example oral, parenteral, nasal, ocular, rectal and topical administration.

Solid compositions for oral administration include tablets, granulates and capsules. In any case the manufacturing method is based on a simple mixture, dry granulation or wet granulation of the active compound with excipients. These excipients can be, for example, diluents such as lactose, microcrystalline cellulose, mannitol or calcium hydrogenphosphate; binding agents such as for example starch, gelatin or povidone; disintegrants such as sodium carboxymethyl starch or sodium croscarmellose; and lubricating agents such as for example magnesium stearate, stearic acid or talc. Tablets can be additionally coated with suitable excipients by using known techniques with the purpose of delaying their disintegration and absorption in the gastrointestinal tract and thereby provide a sustained action over a longer period, or simply to improve their organoleptic properties or their stability. The active compound can also be incorporated by coating onto inert pellets using natural or synthetic film-coating agents. Soft gelatin capsules are also possible, in which the active compound is mixed with water or an oily medium, for example coconut oil, mineral oil or olive oil.

Powders and granulates for the preparation of oral suspensions by the addition of water can be obtained by mixing the active compound with dispersing or wetting agents; suspending agents and preservatives. Other excipients can also be added, for example sweetening, flavouring and colouring agents.

Liquid forms for oral administration include emulsions, solutions, suspensions, syrups and elixirs containing commonly-used inert diluents, such as purified water, ethanol, sorbitol, glycerol, polyethylene glycols (macrogols) and propylene glycol. Said compositions can also contain coadjuvants such as wetting, suspending, sweetening, flavouring agents, preservatives and buffers.

Injectable preparations, according to the present invention, for parenteral administration, comprise sterile solutions, suspensions or emulsions, in an aqueous or non-aqueous solvent such as propylene glycol, polyethylene glycol or vegetable oils. These compositions can also contain coadjuvants, such as wetting, emulsifying, dispersing agents and preservatives. They may be sterilized by any known method or prepared as sterile solid compositions, which will be dissolved in water or any other sterile injectable medium immediately before use. It is also possible to start from sterile materials and keep them under these conditions throughout all the manufacturing process.

For the rectal administration, the active compound can be preferably formulated as a suppository on an oily base, such as for example vegetable oils or solid semisynthetic glycerides, or on a hydrophilic base such as polyethylene glycols (macrogol).

The compounds of the invention can also be formulated for their topical application for the treatment of pathologies occurring in zones or organs accessible through this route, such as eyes, skin and the intestinal tract. Formulations include creams, lotions, gels, powders, solutions and patches wherein the compound is dispersed or dissolved in suitable excipients.

For the nasal administration or for inhalation, the compound can be formulated as an aerosol and it can be conveniently released using suitable propellants.

The dosage and frequency of doses will depend upon the nature and severity of the disease to be treated, the age, the general condition and body weight of the patient, as well as the particular compound administered and the route of administration, among other factors. A representative example of a suitable dosage range is from about 0.01 mg/Kg to about 100 mg/Kg per day, which can be administered as single or divided doses.

The following examples illustrate the scope of the invention.

### EXAMPLES

The following abbreviations have been used in the examples:

AcN: acetonitrile
AcOH: acetic acid
BINAP: 2,2′-bis(diphenylphosphine)-1,1′-binaphthyl
n-BuOH: 1-butanol
CDI: 1,1′-carbonyldiimidazole
d. doublet
dd: double doublet

### DIEA: N,N-diisopropylethylamine

DMAP: 4-(dimethylamino)pyridine
DME: 1,2-dimethoxyethane

### DMF: N,N-dimethylformamide

EDC: N-[3-(dimethylamino)propyl]-N′-ethylcarbodiimide
EtOAc: ethyl acetate
EtOH: ethanol
HBTU: O-Benzotriazol-1-yl-N,N,N′,N′,-tetramethyluronium hexafluorophosphate
HOBT: 1-hydroxybenzotriazole
HPLC: high performance liquid chromatography
LC-MS: liquid chromatography-mass spectroscopy
m: multiplet
MeOH: methanol

### NMM: N-methylmorpholine

NMR: nuclear magnetic resonance
Pd(PPh₃)₄: tetrakis(triphenylphosphine) palladium (0)
Pd₂(dba)₃: tris(dibenzylidenacetone)dipalladium(0)
s: singlet
TEA: triethylamine
THF: tetrahydrofurane
TMS: tetramethylsylane
t_{R}: retention time
X-Phos: 2-dicyclohexylphosphino-2′,4′,6′-triisopropyl-biphenyl

LC-MS spectra have been performed using the following chromatographic methods:

Method 1: Column X-Terra, MS C18 5 μm (100 mm×2.1 mm), temperature: 30° C., flow: 0.35 mL/min, eluent: A=AcN, B=NH₄HCO₃ 10 mM, gradient: 0 min A 10%; 10 min A 90%; 15 min A 90%; 15.01 min A 10%.
Method 2: Column X-bridge, MS C18 2.5 μm (50 mm×2.1 mm), temperature: 50° C., flow: 0.50 mL/min, eluent: A=NH₄HCO₃ 10 mM, B=AcN, C=H₂O, gradient: 0 min A 10%, B 10%; 4 min A 10%, B 85%; 4.75 min A 10%, B 85%; 4.76 min A 10%, B 10%.
Method 3: Column Tracer Excel 120, ODSB 5 μm (10 mm×0.21 mm), temperature: 30° C., flow: 0.35 mL/min, eluent: A=AcN, B=0.1% HCOOH, gradient: 0 min 10% A-10 min 90% A.
Method 4: Column YMC, 3 μm (50 mm×4.6), temperature: 30° C., flow: 2.6 mL/min, eluent: A=H₂O (0.1% HCOOH) B=AcN (0.1% HCOOH), gradient: 0 min 5% B; 4.8 min 95% B; 6 min 95% B.
Method 5: Column Acquity HPLC BEH C18 1.7 μm (2.1×50 mm), temperature: 40° C., flow: 0.50 mL/min, eluent: A=AcN, B=NH₄HCO₃ 10 mM, gradient: 0 min A 10%; 0.25 min A 10%; 3.00 min A 90%; 3.75 min A 90%.

### Reference Example 1

### 2,6-Dichloro-9-(tetrahydropyran-2-yl)-9H-purine

To a suspension of 2,6-dichloropurine (2.00 g, 10.58 mmol) in EtOAc (36 mL) under Ar-atmosphere, 3,4-dihydro-2H-pyrane (2.40 mL, 26.40 mmol) and p-toluensulfonic acid (0.30 g, 1.59 mmol) were added. The resulting mixture was stirred at 57° C. for 4 h. It was allowed to reach room temperature. EtOAc was evaporated. The crude product obtained was chromatographed over silica gel using Hexane/EtOAc mixtures of increasing polarity as eluent, to afford 2.30 g of the title compound (80% yield).

LC-MS (method 1): t_{R}=6.79 min; m/z=271 (MH⁻).

### Reference Example 2

### 2-Chloro-6-(6-fluoropyridin-3-yl)-9-(tetrahydropyran-2-yl)-9H-purine

To a solution of reference example 1 (0.40 g, 1.46 mmol) in DME (14 mL) under Ar-atmosphere, 2-fluoro-5-pyridylboronic acid (0.20 g, 1.46 mmol), Pd(PPh₃)₄ (0.17 g, 0.14 mmol) and a solution of Na₂CO₃ (0.31 g, 2.92 mmol) in H₂O (1.46 mL) were added. The mixture was heated at 90° C. overnight. After cooling, it was diluted with EtOAc and washed thrice with H₂O. The organic phase was dried over Na₂SO₄ and concentrated to dryness. The crude product obtained was chromatographed over silica gel using Hexane/EtOAc mixtures of increasing polarity as eluent, to afford 0.16 g of the title compound (33% yield).

LC-MS (method 1): t_{R}=8.32 min; m/z=334 (MH⁺).

### Reference Example 3

### 2-[4-(tert-Butoxycarbonylamino)piperidin-1-yl]-5-(4,4,5,5-tetramethyl-[1,3,2]dioxaborolan-2-yl)pyridine

### a) 5-Bromo-2-[4-(tert-butoxycarbonylamino)piperidin-1-yl]pyridine

To a suspension of 2,5-dibromopyridine (3.43 g, 14.50 mmol) and DIEA (3.78 mL, 21.70 mmol) in n-BuOH (35 mL), 4-(tert-butoxycarbonylamino)piperidine (3.19 g, 0.06 mmol) was added. The mixture was heated for 48 h at 120° C., cooled and concentrated to dryness. The crude product obtained was chromatographed over silica gel using Hexane/EtOAc mixtures of increasing polarity as eluent, to afford 2.83 g of the desired compound (55% yield).

¹H NMR (300 MHZ, CDCl₃) 8 (TMS): 8.17 (d, J=3.0 Hz, 1H), 7.50 (dd, J=8.8 J=3.0 Hz, 1H), 6.55 (d, J=8.8 Hz, 1H, 1H), 4.45 (broad s, 1H), 4.14 (m, 2H), 3.75 (m, 1H), 2.96 (m, 2H), 2.00 (m, 2H), 1.44 (s, 9H), 1.42 (m, 2H).

### b) Title Compound

To a solution of the compound obtained in the previous section (2.83 g, 7.97 mmol) in DMF (91 mL), bis(pinacolato)diboron (4.05 g, 15.90 mmol), potassium acetate (3.90 g, 39.80 mmol) and [1,1′-bis(diphenylphosphine)ferrocene]dichloropalladium (II) (0.09 g, 0.11 mmol) were added. The reaction mixture was heated at 90° C. overnight. It was cooled until room temperature. DMF was evaporated, the residue was taken up in EtOAc and washed twice with H₂O. The organic phase was dried over Na₂SO₄ and concentrated to dryness. The crude product obtained was chromatographed over silica gel using Hexane/EtOAc mixtures of increasing polarity as eluent, to afford the desired compound in a quantitative yield.

LC-MS (method 2) t_{R}=3.18 min; m/z=404.5 (MH⁻).

### Reference Example 4

### 2-[4-(4-Acetyl-[1,4]diazepan-1-yl)phenyl]-4,4,5,5-tetramethyl-[1,3,2]dioxaborolane

### a) 1-Acetyl-4-(4-bromophenyl)-[1,4]diazepan

To a solution of 1,4-dibromobenzene (3.30 g, 14 mmol) in toluene (44 mL) under Ar-atmosphere, sodium tert-butoxide (1.88 g, 19.60 mol), BINAP (0.17 g, 0.28 mmol), Pd₂(dba)₃ (0.13 g, 0.14 mmol) and 1-acetylhomopiperazine (2 g, 14 mmol) were added at room temperature. The reaction mixture was heated at 80° C. overnight. The resulting mixture was cooled, diluted with MeOH and filtered over Celite®. The filtrate was concentrated to dryness. The crude product obtained was chromatographed over silica gel using Hexane/EtOAc mixtures of increasing polarity as eluent, to afford 3.42 g of the desired compound (82% yield).

LC-MS (method 1): t_{R}=7.08 min; m/z=299 (MH⁺).

### b) Title Compound

Following a similar procedure to that described in reference example 3 section b, but using the compound obtained in the previous section instead of 5-bromo-2-[4-(tert-butoxycarbonylamino)piperidin-1-yl]pyridine, the desired compound was obtained (56% yield).

LC-MS (method 1): t_{R}=7.59 min; m/z=345 (MH⁺).

Following a similar procedure to that described in reference example 4, but using in each case the corresponding starting materials, the following compound was obtained:

| Reference | | Starting | HPLC | t_{R} | |
|---|---|---|---|---|---|
| example | Compound name | material | method | (min) | m/z |
| 4a | 2-{4-[3-(hydroxy- | 3-hydroxy- | 1 | 8.46 | 318 |
| | methyl)piperidin-1- | methyl- | | | |
| | yl]phenyl}-4,4,5,5- | piperidine | | | |
| | tetramethyl- | hydrochloride | | | |
| | [1,3,2]dioxa- | | | | |
| | borolane | | | | |

### Reference Example 5

### 2-[4-(4-tert-Butoxycarbonyl-[1,4]diazepan-1-yl)phenyl]-4,4,5,5-tetramethyl-[1,3,2]dioxaborolane

Following a similar procedure to that described in reference example 4, but using 1-tert-butoxycarbonylhomopiperazine instead of 1-acetylhomopiperazine, the desired compound was obtained (16% yield).

LC-MS (method 1): t_{R}=10.97 min; m/z=403 (MH⁺).

### Reference Example 6

### 2-{4-[((4-tert-Butoxycarbonyl)piperazin-1-yl)sulfonyl]phenyl}-4,4,5,5-tetramethyl-[1,3,2]dioxaborolane

### a) 4-Bromo-1-[((4-tert-butoxycarbonyl)piperazin-1-yl)sulfonyl]benzene

To a solution of 4-bromobenzenesulfonyl chloride (10 g, 39.13 mmol) in pyridine (120 mL), DMAP (1 mg) and 1-tert-butoxycarbonylpiperazine (7.20 g, 39.13 mmol) were added. The mixture was stirred at 60° C. for 18 h. It was cooled until room temperature and evaporated. The residue was washed with a saturated aqueous solution of NaHCO₃ and extracted thrice with EtOAc. The organic phase was dried over Na₂SO₄ and concentrated to dryness. The crude product obtained was chromatographed over silica gel using Hexane/EtOAc mixtures of increasing polarity as eluent, to afford 8.50 g of the desired compound (53% yield).

### b) Title Compound

Following a similar procedure to that described in reference example 3 section b, but using the compound obtained in the previous section instead of 5-bromo-2-[4-(tert-butoxycarbonylamino)piperidin-1-yl]pyridine, the desired compound was obtained (63% yield).

LC-MS (method 1): t_{R}=6.64 min; m/z=369 (MH⁻).

### Reference Example 7

### 3-Amino-N-(2-hydroxyethyl)benzenesulfonamide

### a) N-(2-hydroxyethyl)-3-nitrobenzenesulfonamide

To a solution of 3-nitrobenzenesulfonyl chloride (0.50 g, 2.25 mmol) in THF (5 mL), 2-aminoethanol (1.83 mL, 30.38 mmol) was added. The mixture was stirred at room temperature for 18 h. It was diluted with EtOAc and washed thrice with 0.5 N HCl. The organic phase was dried over Na₂SO₄ and concentrated to dryness. The crude product thus obtained was directly used in the next step.

### b) Title Compound

To a solution of the compound obtained in the previous section (0.64 g, 2.60 mmol) in MeOH (15 mL) under Ar-atmosphere, 10% Pd/C (64 mg) was added at room temperature. The resulting mixture was stirred under H₂ overnight, filtered and the filtrate was concentrated to dryness. The crude product thus obtained was chromatographed over silica gel using Hexane/EtOAc mixtures of increasing polarity as eluent, to afford 0.39 g of the desired compound (69% yield).

LC-MS (method 1): t_{R}=2.21 min; m/z=217 (MH⁺).

### Reference Example 8

### [4-(3-Hydroxypiperidin-1-yl)phenyl]amine

### a) 4-(3-Hydroxypiperidin-1-yl)nitrobenzene

To a solution of 4-fluoronitrobenzene (1 g, 7.09 mmol) in AcN (16 mL), 3-hydroxypiperidine hydrochloride (1.04 g, 7.57 mmol) and DIEA (1.32 mL, 7.57 mmol) were added. The mixture was stirred and refluxed for 18 h. The resulting mixture was cooled until room temperature and evaporated. The crude product thus obtained was chromatographed over silica gel using Hexane/EtOAc mixtures of increasing polarity as eluent, to afford 1.15 g of the desired compound (51% yield).

### b) Title Compound

Following a similar procedure to that described in reference example 7 section b, but using the compound obtained in the previous section, the desired compound was obtained in quantitative yield.

LC-MS (method 1): t_{R}=3.06 min; m/z=193 (MH⁺).

Following a similar procedure to that described in reference example 8, but using in each case the corresponding starting materials, the following compounds were obtained:

| Reference | | | HPLC | t_{R} | |
|---|---|---|---|---|---|
| Example | Compound name | Starting material | method | (min) | m/z |
| 8a | [4-(3-tert- | 3-tert- | 1 | 5.41 | 308 |
| | butoxycarbonylaminopyrrolidin- | butoxycarbonylaminopyrrolidine | | | |
| | 1-yl)phenyl]amine | | | | |
| 8b | [4-(3-hydroxypyrrolidin-1- | 3- | 1 | 2.53 | 179 |
| | yl)phenyl]amine | hydroxypyrrolidine | | | |
| 8c | [4-(3-tert- | 3-tert- | 1 | 6.67 | 292 |
| | butoxycarbonylaminopiperidin-1- | butoxycarbonylaminopiperidine | | | |
| | yl)phenyl]amine | | | | |
| 8d | [4-(3-(S)-hydroxypiperidin-1- | 3-(S)- | 1 | 5.94 | 223 |
| | yl)phenyl]amine | hydroxypiperidine | | | |
| | | hydrochloride (1) | | | |
| 8e | [4-(3-(R)-Hydroxypiperidin-1- | 3-(R)- | 1 | 5.94 | 223 |
| | yl)phenyl]amine | hydroxypiperidine | | | |
| | | hydrochloride (1) | | | |
| 8f | [4-(cis-3,5-dimethylpiperazin-1- | cis-2,6- | 5 | 0.56 | 206 |
| | yl)phenyl]amine | dimethylpiperazine | | | |

(1) Step b was performed as described below in reference example 10 section b.

### Reference Example 9

### 2-{4-[(S)-3-hydroxypiperidin-1-yl]phenyl}-4,4,5,5-tetramethyl-[1,3,2]dioxaborolane

### a) 4-Bromo-1-(3-(S)-hydroxypiperidin-1-yl)benzene

To a solution of reference example 8d (0.37 g, 1.92 mmol) in 8.2 mL HBr 48%, a solution of NaNO₂ (0.133 g, 1.92 mmol) in 1.4 mL of H₂O was slowly added at 0° C. The mixture was stirred for 15 minutes and added to a solution of CuBr (0.151 g, 1.06 mmol) in 2.7 mL HBr 48%. The resulting mixture was stirred and refluxed for 2 h. The suspension thus obtained was partitioned between 2N NaOH and ethyl acetate. The organic layer was washed with aqueous NaCl, dried over Na₂SO₄ and concentrated to dryness. The desired compound was obtained (0.387 g, 84%).

### b) Title Compound

Following a similar procedure to that described in reference example 3 section b, but using the compound obtained in the previous section instead of 5-bromo-2-[4-(tert-butoxycarbonylamino)piperidin-1-yl]pyridine, the desired compound was obtained (71% yield).

LC-MS (method 1): t_{R}=8.02 min; m/z=304 (MH⁺).

### Reference Example 10

### 3-(4-Aminophenyl)-1-[(2-(trimethylsilyl)ethoxy)methyl]-1H-pyrazole

### a) 3-(4-Nitrophenyl)-1-[(2-(trimethylsilyl)ethoxy)methyl]-1H-pyrazole

To a solution of 3-(4-nitrophenyl)pyrazole (200 mg, 1.06 mmol) in CHCl₃ (3 mL) and DIEA (0.55 mL, 3.18 mmol) under Ar-atmosphere, 2-(trimethylsilyl)-ethoxymethyl chloride (282 μL, 1.59 mmol) was added at 0° C. The resulting mixture was stirred at room temperature overnight. Water was added and the phases were separated. The aqueous layer was extracted twice with CHCl₃. The combined organic phases were dried over Na₂SO₄ and concentrated to dryness. The crude product thus obtained was chromatographed over silica gel using hexane/EtOAc mixtures of increasing polarity as eluent, to afford the desired compound in quantitative yield.

LC-MS: (method 1): t_{R}=10.51 min; m/z=320 (MH⁺)

### b) Title Compound

To a solution of the compound obtained in the previous section (350 mg, 1.08 mmol) and NiCl₂.6H₂O (104 mg, 044 mmol) in MeOH/THF (27 mL/14 mL), NaBH₄ (175 mg, 4.62 mmol) was added. The resulting mixture was stirred for 1 h at room temperature. The mixture was partitioned between 1N NaOH and ethyl acetate, and the organic layer was washed with aqueous NaCl and dried over Na₂SO₄. The crude product thus obtained was chromatographed over silica gel using hexane/EtOAc mixtures of increasing polarity as eluent, to afford the desired compound in quantitative yield.

LC-MS (method 1): t_{R}=8.54 min; m/z=290 (MH⁺).

### Reference Example 11

### 2-[2-Methoxycarbonyl-1-(4-toluoyl)sulfonylpyrrole-4-yl]-4,4,5,5-tetramethyl-[1,3,2]dioxaborolane

### a) 4-Iodo-2-methyloxycarbonyl-1-(4-toluoyl)sulfonylpyrrole

To a solution of 4-iodo-2-methoxycarbonylpyrrole (4.576 g, 18.3 mmol) in 50 mL dichloromethane was added triethylamine (5.7 mL, 40.10 mmol), N,N-dimethylaminopyridine (0.245 g, 2.00 mmol), and p-toluoylsulfonyl chloride (3.823 g, 20.05 mmol). The mixture was stirred at room temperature overnight. The solution was consecutively washed with 1N HCl, NaHCO₃ saturated aqueous solution, and NaCl saturated solution. The organic layer was dried over Na₂SO₄ and concentrated to dryness. The resulting crude product was recrystallized in tert-buthylmethylether to obtain 4.562 g (62% yield) of the title compound as a yellow solid.

### b) Title Compound

To a solution of the compound obtained in the previous section (1.00 g, 2.47 mmol) in DMF (30 mL), bis(pinacolato)diboron (1.25 g, 4.92 mmol), potassium acetate (1.21 g, 12.34 mmol) and [1,1′-bis(diphenylphosphine)ferrocene]dichloropalladium (II) (0.20 g, 0.245 mmol) were added. The reaction mixture was heated at 95° C. overnight under an Ar-atmosphere. The mixture was cooled to room temperature, the solvent was evaporated, and the residue was triturated with 200 mL diethyl ether. The resulting suspension was filtered and evaporated to dryness. The crude product thus obtained was chromatographed over silica gel using hexane/EtOAc mixtures of increasing polarity as eluent, to afford 0.86 g of the desired compound (86% yield).

LC-MS (method 5) t_{R}=2.95 min; m/z=405 (MH⁺).

### Reference Example 12

### 4-(2-Hydroxy-2-methylpropyl)phenylamine

### a) Ethyl 4-benzyloxycarbonylaminophenylacetate

To a solution of ethyl 4-aminophenylacetate (1.00 g, 5.5 mmol) and Na₂CO₃ (0.77 g, 7.2 mmol) in H₂O:THF (10 mL:3 mL), benzyl chloroformiate (0.8 mL, 5.5 mmol) was added. The mixture was stirred at room temperature overnight. The resulting mixture was diluted with dichloromethane (50 mL) and partitioned between H₂O and dichloromethane. The organic phase was dried over Na₂SO₄ and concentrated to dryness. The crude product thus obtained was chromatographed over silica gel using hexane/EtOAc mixtures of increasing polarity as eluent, to afford 1.1 g of the desired compound.

### b) Benzyl 4-(2-hydroxy-2-methylpropyl)phenylcarbamate

To a solution of the compound obtained in the previous section (1.1 g, 3.537 mmol) in THF (30 mL) at 0° C., a solution of methylmagnesium bromide (12.2 mL, 3M in diethyl ether) was added. The resulting mixture was stirred for 1 h at room temperature and the resulting suspension was evaporated to dryness. The crude product thus obtained was chromatographed over silica gel using hexane/EtOAc mixtures of increasing polarity as eluent, to afford 0.87 g of the desired compound (82% yield).

### c) Title Compound

Following a similar procedure to that described in reference example 7 section b, but using the compound obtained in the previous section, the desired compound was obtained in quantitative yield.

LC-MS (method 5): t_{R}=1.19 min; m/z=166 (MH⁺).

### Reference Example 13

### N-(3-Aminophenyl)-N-methylacetamide

### a) N-(3-Nitrophenyl)-N-methylacetamide

To a solution of 3-nitro-N-methylaniline (650 mg, 4.27 mmol) in CH₂Cl₂ (10 mL) under Ar-atmosphere, acetyl chloride (0.33 mL, 4.7 mmol), a catalytic amount of DMAP and DIEA (1.49 mL, 8.5 mmol) were added. The resulting mixture was stirred at room temperature overnight. The resulting residue was diluted with H₂O, the phases were separated and the aqueous phase extracted with CH₂Cl₂. The combined organic phases were dried over Na₂SO₄ and concentrated to dryness. The crude product thus obtained was directly used in the next step.

LC-MS (method 5): t_{R}=1.43 min; m/z=195 (MH⁺).

### b) Title Compound

Following a similar procedure to that described in reference example 7 section b, but using the compound obtained in the previous section, the desired compound was obtained (65% yield).

LC-MS (method 5): t_{R}=1.02 min; m/z=165 (MH⁺).

Following a similar procedure to that described in reference example 13, but using in each case the corresponding starting materials, the following compounds were obtained:

| Refer- | | | | | |
|---|---|---|---|---|---|
| ence | | reagent for | HPLC | t_{R} | |
| example | Compound name | step a) | method | (min) | m/z |
| 13a | N-(3-aminophenyl)-N- | Isobutyryl | 5 | 1.89 | 193 |
| | isopropylacetamide | chloride | | | |
| 13b | N-(3-aminophenyl)-N- | Cyclopropane | 5 | 1.38 | 191 |
| | cyclopropylacetamide | carbonyl | | | |
| | | chloride | | | |

### Reference Example 14

### 2-[1-(Methanesulfonyl)-1H-indol-5-yl]-4,4,5,5-tetramethyl-[1,3,2]dioxaborolane

### a) 5-Bromo-1-(methanesulfonyl)-1H-indole

To a solution of 5-bromoindole (1 g, 5.1 mmol) in THF (10 mL) under Ar-atmosphere, methanesulfonyl chloride (0.88 mL, 12.75 mmol), and TEA (2.23 mL, 15.3 mmol) were added. The mixture was stirred at room temperature overnight. The resulting mixture was evaporated and the crude product thus obtained was directly used in the next step.

LC-MS (method 4): t_{R}=3.30 min; m/z=276 (MH⁺).

### b) Title Compound

Following a similar procedure to that described in reference example 3 section b, but using the compound obtained in the previous section instead of 5-bromo-2-[4-(tent-butoxycarbonylamino)piperidin-1-yl]pyridine, the desired compound was obtained (56% yield).

LC-MS (method 4): t_{R}=3.68 min; m/z=322 (MH⁺).

### Example 1

### 6-[6-(4-Aminopiperidin-1-yl)pyridin-3-yl]-2-[4-(4-morpholino)phenyl]amino-9H-purine

### a) 6-[6-[4-(tert-Butoxycarbonyl)aminopiperidin-1-yl]pyridin-3-yl]-2-chloro-9-(tetrahydropyran-2-yl)-9H-purine

Following a similar procedure to that described in reference example 2, but using the compound obtained in reference example 3 instead of 2-fluoro-5-pyridylboronic acid, the desired compound was obtained (93% yield).

LC-MS (method 1): t_{R}=9.72 min; m/z=514 (MH⁺).

### b) 6-{6-[4-(tert-Butoxycarbonyl)aminopiperidin-1-yl]pyridin-3-yl}-2-[4-(4-morpholino)phenyl]amino-9-(tetrahydropyran-2-yl)-9H-purine

To a solution of the compound obtained in the previous section (70 mg, 0.136 mmol) in toluene (1.75 mL) under Ar-atmosphere, sodium tert-butoxide (18 mg, 0.190 mmol), BINAP (7 mg, 0.010 mmol), Pd₂(dba)₃ (16 mg, 0.005 mmol) and [4-(4-morpholino)phenyl]amine (36 mg, 0.200 mmol) were added at room temperature. The reaction mixture was heated at 100° C. overnight. It was diluted with EtOAc and washed thrice with H₂O. The organic phase was dried over Na₂SO₄ and concentrated to dryness. The crude product obtained was chromatographed over silica gel using Hexane/EtOAc mixtures of increasing polarity as eluent, to afford 63 mg of the desired compound (71% yield).

LC-MS (method 1): t_{R}=9.52 min; m/z=656 (MH⁺)

### c) Title Compound

In a flask were mixed, under Ar-atmosphere, the compound obtained in the previous section (63 mg, 0.09 mmol) and a mixture of 4M dioxane/HCl_{(g)} (3 mL). It was stirred at room temperature overnight and concentrated to dryness. The resulting residue was washed with a 1N NaOH and extracted with CHCl₃. The organic phase was dried over Na₂SO₄ and concentrated to dryness. The crude product obtained was chromatographed over silica gel using CHCl₃/MeOH/NH₃ mixtures of increasing polarity as eluent, to afford 26 mg of the desired compound (58% yield).

LC-MS (method 1): t_{R}=4.95 min; m/z=472 (MH⁺).

Following a similar procedure to that described in example 1, but using in each case the corresponding starting materials, these compounds were obtained:

| Exam- | | Reagent | Reagent for | HPLC | t_{R} | |
|---|---|---|---|---|---|---|
| ple | Compound name | for step a) | step b) | method | (min) | m/z |
| 1a | 6-[6-(4-aminopiperidin-1- | Reference | (4- | 1 | 4.72 | 465 |
| | yl)pyridin-3-yl]-2-(4- | Example 3 | methanesulfonylphenyl)amine | | | |
| | methanesulfonylphenyl)amino- | | | | | |
| | 9H-purine | | | | | |
| 1b | 2-(4- | Reference | N-(4- | 1 | 4.41 | 444 |
| | acetamidophenyl)amino-6- | Example 3 | aminophenyl)acetamide | | | |
| | [6-(4-aminopiperidin-1- | | | | | |
| | yl)pyridin-3-yl]-9H-purine | | | | | |
| 1c | 2-(4-acetylphenyl)amino-6- | Reference | 4′- | 1 | 5.23 | 429 |
| | [6-(4-aminopiperidin-1- | Example 3 | aminoacetophenone | | | |
| | yl)pyridin-3-yl]-9H-purine | | | | | |
| 1d | 6-[6-(4-aminopiperidin-1- | Reference | (4- | 1 | 6.21 | 433 |
| | yl)pyridin-3-yl]-2-(4- | Example 3 | methylsulfanylphenyl)amine | | | |
| | methylsulfanylphenyl)amino- | | | | | |
| | 9H-purine | | | | | |
| 1e | 6-[4-(4-acetyl- | Reference | 2-aminothiazole | 1 | 5.56 | 435 |
| | [1,4]diazepan-1-yl)phenyl]- | Example 4 | | | | |
| | 2-(2-thiazolyl)amino-9H- | | | | | |
| | purine | | | | | |
| 1f | 6-[6-(4-aminopiperidin-1- | Reference | 4- | 1 | 5.42 | 431 |
| | yl)pyridin-3-yl]-2-(4- | Example 3 | aminobenzylic | | | |
| | methoxymethylphenyl)amino- | | alcohol | | | |
| | 9H-purine | | | | | |
| 1g | 6-[6-(4-aminopiperidin-1- | Reference | 4-aminophenol | 1 | 4.49 | 403 |
| | yl)pyridin-3-yl]-2-(4- | Example 3 | | | | |
| | hydroxyphenyl)amino-9H- | | | | | |
| | purine | | | | | |
| 1h | 6-[6-(4-aminopiperidin-1- | Reference | (4- | 1 | 7.00 | 519 |
| | yl)pyridin-3-yl]-2-(4- | Example 3 | trifluoromethanesulfonylphenyl)amine | | | |
| | trifluoromethanesulfonylphenyl)amino- | | | | | |
| | 9H-purine | | | | | |
| 1i | 6-[6-(4-aminopiperidin-1- | Reference | 4- | 1 | 5.55 | 412 |
| | yl)pyridin-3-yl]-2-(4- | Example 3 | aminobenzonitrile | | | |
| | cyanophenyl)amino-9H- | | | | | |
| | purine | | | | | |
| 1j | 6-[6-(4-aminopiperidin-1- | Reference | [3-(1- | 1 | 6.63 | 470 |
| | yl)pyridin-3-yl]-2-[3-(1- | Example 3 | piperidinyl)phenyl]amine | | | |
| | piperidinyl)phenyl]amino- | | | | | |
| | 9H-purine | | | | | |
| 1k | 6-[6-(4-aminopiperidin-1- | Reference | [4-(1- | 3 | 3.41 | 470 |
| | yl)pyridin-3-yl]-2-[4-(1- | Example 3 | piperidinyl)phenyl]amine | | | |
| | piperidinyl)phenyl]amino- | | | | | |
| | 9H-purine | | | | | |
| 1l | 6-[6-(4-aminopiperidin-1- | Reference | 4- | 1 | 6.59 | 491 |
| | yl)pyridin-3-yl]-2-(4- | Example 3 | aminobenzophenone | | | |
| | benzoylphenyl)amino-9H- | | | | | |
| | purine | | | | | |
| 1m | 6-[6-(4-aminopiperidin-1- | Reference | 2-(4- | 1 | 4.71 | 431 |
| | yl)pyridin-3-yl]-2-[4-(2- | Example 3 | aminophenyl)ethanol | | | |
| | hydroxyethyl)phenyl]amino- | | | | | |
| | 9H-purine | | | | | |
| 1n | 6-[6-(4-aminopiperidin-1- | Reference | [4-(4- | 3 | 2.80 | 485 |
| | yl)pyridin-3-yl]-2-[4-(4- | Example 3 | methylpiperazin- | | | |
| | methylpiperazin-1- | | 1- | | | |
| | yl)phenyl]amino-9H-purine | | yl)phenyl]amine | | | |
| 1o | 6-[6-(4-aminopiperidin-1- | Reference | (3- | 1 | 4.34 | 465 |
| | yl)pyridin-3-yl]-2-(3- | Example 3 | methanesulfonylphenyl)amine | | | |
| | methanesulfonylphenyl)amino- | | | | | |
| | 9H-purine | | | | | |
| 1p | 6-[6-(4-aminopiperidin-1- | Reference | [3-(4- | 1 | 5.79 | 464 |
| | yl)pyridin-3-yl]-2-[3-(4- | Example 3 | pyridyl)phenyl]amine | | | |
| | pyridyl)phenyl]amino-9H- | | | | | |
| | purine | | | | | |
| 1q | 2-(3- | Reference | N-(3- | 1 | 4.67 | 444 |
| | acetamidophenyl)amino-6- | Example 3 | aminophenyl)acetamide | | | |
| | [6-(4-aminopiperidin-1- | | | | | |
| | yl)pyridin-3-yl]-9H-purine | | | | | |
| 1r | 6-[6-(4-aminopiperidin-1- | Reference | N-(3- | 1 | 4.81 | 480 |
| | yl)pyridin-3-yl]-2-(3- | Example 3 | aminophenyl)methylsulfonamide | | | |
| | methylsulfonamidophenyl)amino- | | | | | |
| | 9H-purine | | | | | |
| 1s | 6-[6-(4-aminopiperidin-1- | Reference | 3-amino-N-tert- | 1 | 4.50 | 466 |
| | yl)pyridin-3-yl]-2-(3- | Example 3 | butylbenzenesulfonamide | | | |
| | aminosulfonylphenyl)amino- | | | | | |
| | 9H-purine | | | | | |
| 1t | 6-[6-(4-aminopiperidin-1- | Reference | 4-amino-N-tert- | 1 | 4.39 | 466 |
| | yl)pyridin-3-yl]-2-(4- | Example 3 | butylbenzenesulfonamide | | | |
| | aminosulfonylphenyl)amino- | | | | | |
| | 9H-purine | | | | | |
| 1u | 6-[6-(4-aminopiperidin-1- | Reference | (3- | 1 | 6.23 | 433 |
| | yl)pyridin-3-yl]-2-(3- | Example 3 | methylsulfanylphenyl)amine | | | |
| | methylsulfanylphenyl)amino- | | | | | |
| | 9H-purine | | | | | |
| 1v | 6-[6-(4-aminopiperidin-1- | Reference | 3- | 1 | 6.66 | 491 |
| | yl)pyridin-3-yl]-2-(3- | Example 3 | aminobenzophenone | | | |
| | benzoylphenyl)amino-9H- | | | | | |
| | purine | | | | | |
| 1w | 6-[6-(4-aminopiperidin-1- | Reference | (3- | 1 | 7.36 | 493 |
| | yl)pyridin-3-yl]-2-(3- | Example 3 | benzyloxyphenyl)amine | | | |
| | benzyloxyphenyl)amino- | | | | | |
| | 9H-purine | | | | | |
| 1x | 6-[6-(4-aminopiperidin-1- | Reference | (3- | 1 | 6.76 | 478 |
| | yl)pyridin-3-yl]-2-(3- | Example 3 | phenylaminophenyl)amine | | | |
| | phenylaminophenyl)amino- | | | | | |
| | 9H-purine | | | | | |
| 1y | 6-[6-(4-aminopiperidin-1- | Reference | [4-(1- | 3 | 2.50 | 471 |
| | yl)pyridin-3-yl]-2-[4-(1- | Example 3 | piperazinyl)phenyl]amine | | | |
| | piperazinyl)phenyl]amino- | | | | | |
| | 9H-purine | | | | | |
| 1z | 6-[6-(4-aminopiperidin-1- | Reference | [3-(1- | 1 | 5.86 | 471 |
| | yl)pyridin-3-yl]-2-[3-(1- | Example 3 | piperazinyl)phenyl]amine | | | |
| | piperazinyl)phenyl]amino- | | | | | |
| | 9H-purine | | | | | |
| 1aa | 6-[6-(4-aminopiperidin-1- | Reference | (3,4,5- | 1 | 5.30 | 477 |
| | yl)pyridin-3-yl]-2-(3,4,5- | Example 3 | trimethoxyphenyl)amine | | | |
| | trimethoxyphenyl)amino- | | | | | |
| | 9H-purine | | | | | |
| 1ab | 6-[6-(4-aminopiperidin-1- | Reference | (3,4- | 1 | 5.08 | 447 |
| | yl)pyridin-3-yl]-2-(3,4- | Example 3 | dimethoxyphenyl)amine | | | |
| | dimethoxyphenyl)amino- | | | | | |
| | 9H-purine | | | | | |
| 1ac | 6-[6-(4-aminopiperidin-1- | Reference | 4-amino-N,N- | 1 | 5.54 | 494 |
| | yl)pyridin-3-yl]-2-[3-(N,N- | Example 3 | dimethylaminobenzenesulfonamide | | | |
| | dimethylaminosulfonyl)phenyl]amino- | | | | | |
| | 9H-purine | | | | | |
| 1ad | 6-[6-(4-aminopiperidin-1- | Reference | Reference | 1 | 4.58 | 510 |
| | yl)pyridin-3-yl]-2-{3-[N-(2- | Example 3 | Example 7 | | | |
| | hydroxyethyl)aminosulfonyl]phenyl}- | | | | | |
| | amino-9H-purine | | | | | |
| 1ae | 6-[6-(4-aminopiperidin-1- | Reference | 3-amino-N- | 1 | 4.98 | 480 |
| | yl)pyridin-3-yl]-2-[3-(N- | Example 3 | methylbenzene | | | |
| | methylaminosulfonyl)phenyl]amino- | | sulfonamide | | | |
| | 9H-purine | | | | | |
| 1af | 6-[6-(4-aminopiperidin-1- | Reference | 4-amino-N- | 1 | 4.91 | 480 |
| | yl)pyridin-3-yl]-2-[4-(N- | Example 3 | methylbenzene | | | |
| | methylaminosulfonyl)phenyl]amino- | | sulfonamide | | | |
| | 9H-purine | | | | | |
| 1ag | 6-[6-(4-aminopiperidin-1- | Reference | (3,5- | 1 | 5.81 | 447 |
| | yl)pyridin-3-yl]-2-(3,5- | Example 3 | dimethoxyphenyl)amine | | | |
| | dimethoxyphenyl)amino- | | | | | |
| | 9H-purine | | | | | |
| 1ah | 6-[6-(4-aminopiperidin-1- | Reference | [4-(1,1- | 1 | 4.83 | 520 |
| | yl)pyridin-3-yl]-2-[4-(1,1- | Example 3 | dioxothiomorpholin- | | | |
| | dioxothiomorpholin-4- | | 4- | | | |
| | yl)phenyl]amino-9H-purine | | yl)phenyl]amine | | | |
| 1ai | 6-[6-(4-aminopiperidin-1- | Reference | [4-(N,N- | 1 | 6.62 | 458 |
| | yl)pyridin-3-yl]-2-[4-(N,N- | Example 3 | diethylamino)phenyl]amine | | | |
| | diethylamino)phenyl]amino- | | | | | |
| | 9H-purine | | | | | |
| 1aj | 6-[6-(4-aminopiperidin-1- | Reference | 4-amino-N-(2- | 1 | 4.49 | 510 |
| | yl)pyridin-3-yl]-2-{4-[N-(2- | Example 3 | hydroxyethyl)benzenesulfonamide | | | |
| | hydroxyethyl)aminosulfonyl]phenyl}- | | | | | |
| | amino-9H-purine | | | | | |
| 1ak | 6-[6-(4-aminopiperidin-1- | Reference | 3- | 1 | 5.85 | 412 |
| | yl)pyridin-3-yl]-2-(3- | Example 3 | aminobenzonitrile | | | |
| | cyanophenyl)amino-9H- | | | | | |
| | purine | | | | | |
| 1al | 2-(4- | 5- | 3-amino-N-tert- | 4 | 2.13 | 406 |
| | aminosulfonylphenyl)amino- | indolylboronic | butylbenzenesulfonamide | | | |
| | 6-(1H-indol-5-yl)-9H- | acid | | | | |
| | purine | | | | | |
| 1am | 6-[6-(4-aminopiperidin-1- | Reference | (3- | 1 | 7.34 | 477 |
| | yl)pyridin-3-yl]-2-(3- | Example 3 | benzylphenyl)amine | | | |
| | bencylphenyl)amino-9H- | | | | | |
| | purine | | | | | |
| 1an | 6-[6-(4-aminopiperidin-1- | Reference | Reference | 1 | 4.88 | 486 |
| | yl)pyridin-3-yl]-2-[4-(3- | Example 3 | Example 8 | | | |
| | hydroxypiperidin-1- | | | | | |
| | yl)phenyl]amino-9H-purine | | | | | |
| 1ao | 2-(3-acetylphenyl)amino-6- | Reference | 3′- | 1 | 5.37 | 429 |
| | [6-(4-aminopiperidin-1- | Example 3 | aminoacetophenone | | | |
| | yl)pyridin-3-yl]-9H-purine | | | | | |
| 1ap | 6-[4-(4-acetyl- | Reference | (3,4- | 1 | 6.69 | 472 |
| | [1,4]diazepan-1-yl)phenyl]- | Example 4 | methylendioxyphenyl)amine | | | |
| | 2-(3,4- | | | | | |
| | methylendioxyphenyl)amino- | | | | | |
| | 9H-purine | | | | | |
| 1aq | 6-[4-(4-acetyl- | Reference | N-tert- | 1 | 5.41 | 511 |
| | [1,4]diazepan-1-yl)phenyl]- | Example 4 | butoxycarbonyl- | | | |
| | 2-[4-(piperidin-3- | | 3-(4- | | | |
| | yl)phenyl]amino-9H-purine | | aminophenyl)piperidine | | | |
| 1ar | 6-[4-(4-acetyl- | Reference | Reference | 1 | 5.26 | 512 |
| | [1,4]diazepan-1-yl)phenyl]- | Example 4 | Example 8a | | | |
| | 2-[4-(3-aminopyrrolidin-1- | | | | | |
| | yl)phenyl]amino-9H-purine | | | | | |
| 1as | 6-[4-([1,4]diazpan-1- | Reference | [4-(4- | 1 | 5.67 | 471 |
| | yl)phenyl]-2-[4-(4- | Example 5 | morpholino)phenyl]amine | | | |
| | morpholino)phenyl]amino- | | | | | |
| | 9H-purine | | | | | |
| 1at | 6-[4-(4-acetyl- | Reference | [4-(4- | 1 | 5.91 | 513 |
| | [1,4]diazepan-1-yl)phenyl]- | Example 4 | morpholino)phenyl]amine | | | |
| | 2-[4-(4- | | | | | |
| | morpholino)phenyl]amino- | | | | | |
| | 9H-purine | | | | | |
| 1au | 6-[4-(4-acetyl- | Reference | Reference | 1 | 5.62 | 527 |
| | [1,4]diazepan-1-yl)phenyl]- | Example 4 | Example 8 | | | |
| | 2-[4-(3-hydroxypiperidin-1- | | | | | |
| | yl)phenyl]amino-9H-purine | | | | | |
| 1av | 2-(3-acetylphenyl)amino-6- | 3- | 3′- | 4 | 2.90 | 360 |
| | (3-methoxyphenyl)-9H- | methoxyphenylboronic | aminoacetophenone | | | |
| | purine | acid | | | | |
| 1aw | 6-[4-(4-acetyl- | Reference | 3- | 1 | 5.21 | 429 |
| | [1,4]diazepan-1-yl)phenyl]- | Example 4 | aminopyridine | | | |
| | 2-(3-pyridyl)amino-9H- | | | | | |
| | purine | | | | | |
| 1ax | 6-[4-(4-acetyl- | Reference | 4- | 1 | 5.10 | 429 |
| | [1,4]diazepan-1-yl)phenyl]- | Example 4 | aminopyridine | | | |
| | 2-(4-pyridyl)amino-9H- | | | | | |
| | purine | | | | | |
| 1ay | 2-(4- | 3- | 4-amino-N-tert- | 4 | 3.85 | 435 |
| | aminosulfonylphenyl)amino- | trifluoromethylphenyl | butylbenzenesulfonamide | | | |
| | 6-(3- | boronic | | | | |
| | trifluoromethylphenyl)-9H- | acid | | | | |
| | purine | | | | | |
| 1az | 2-(4- | 3- | 4-amino-N-tert- | 4 | 2.33 | 397 |
| | aminosulfonylphenyl)amino- | methoxyphenylboronic | butylbenzenesulfonamide | | | |
| | 6-(3-methoxyphenyl)-9H- | acid | | | | |
| | purine | | | | | |
| 1ba | 2-(4- | 3- | 4-amino-N-tert- | 4 | 2.68 | 401 |
| | aminosulfonylphenyl)amino- | chlorophenylboronic | butylbenzenesulfonamide | | | |
| | 6-(3-chlorophenyl)-9H- | acid | | | | |
| | purine | | | | | |
| 1bb | 6-[6-(4-aminopiperidin-1- | Reference | 6-amino-2- | 1 | 4.93 | 442 |
| | yl)pyridin-3-yl]-2-(2- | Example 3 | methylbenzooxazol | | | |
| | methylbenzooxazol-6- | | | | | |
| | yl]amino-9H-purine | | | | | |
| 1bc | 6-[4-(N- | 6-[4-(N- | 3-amino-N-tert- | 4 | 1.92 | 424 |
| | acetyl)aminophenyl]-2-(3- | acetyl)aminophenyl]boronic | butylbenzenesulfonamide | | | |
| | aminosulfonylphenyl)amino- | acid | | | | |
| | 9H-purine | | | | | |
| 1bd | 2-[4-(3-hydroxypyrrolidin-1- | 3- | Reference | 4 | 2.12 | 403 |
| | yl)phenyl]amino-6-(3- | methoxyphenylboronic | Example 8b | | | |
| | methoxyphenyl)-9H-purine | acid | | | | |
| 1be | 2-(3- | 3- | (3- | 4 | 3.42 | 409 |
| | phenylaminophenyl)amino- | methoxyphenylboronic | phenylaminophenyl)amine | | | |
| | 6-(3-methoxyphenyl)-9H- | acid | | | | |
| | purine | | | | | |
| 1bf | 2-[4-(3-aminopyrrolidin-1- | 3- | Reference | 4 | 1.72 | 402 |
| | yl)phenyl]amino-6-(3- | methoxyphenylboronic | Example 8a | | | |
| | methoxyphenyl)-9H-purine | acid | | | | |
| 1bg | 2-[4-(3-hydroxypiperidin-1- | 3- | Reference | 4 | 1.73 | 417 |
| | yl)phenyl]amino-6-(3- | methoxyphenylboronic | Example 8 | | | |
| | methoxyphenyl)-9H-purine | acid | | | | |
| 1bh | 2-(3- | 3- | (3- | 4 | 3.88 | 447 |
| | phenylaminophenyl)amino- | trifluoromethylphenyl | phenylaminophenyl)amine | | | |
| | 6-(3-trifluoromethylphenyl)- | boronic | | | | |
| | 9H-purine | acid | | | | |
| 1bi | 2-(3- | 3- | (3- | 4 | 3.37 | 385 |
| | phenylaminophenyl)amino- | thiophenboronic | phenylaminophenyl)amine | | | |
| | 6-(thien-3-yl)-9H-purine | acid | | | | |
| 1bj | 2-(3- | 3- | 3-amino-N-tert- | 4 | 2.38 | 397 |
| | aminosulfonylphenyl)amino- | methoxyphenylboronic | butylbenzenesulfonamide | | | |
| | 6-(3-methoxyphenyl)-9H- | acid | | | | |
| | purine | | | | | |
| 1bk | 2-(3- | 3- | 3-amino-N-tert- | 4 | 2.87 | 435 |
| | aminosulfonylphenyl)amino- | trifluoromethylphenyl | butylbenzenesulfonamide | | | |
| | 6-(3- | boronic | | | | |
| | trifluoromethylphenyl)-9H- | acid | | | | |
| | purine | | | | | |
| 1bl | 6-(1H-indol-5-yl)-2-(3- | 5- | (3- | 4 | 3.05 | 418 |
| | phenylaminophenyl)amino- | indolylboronic | phenylaminophenyl)amine | | | |
| | 9H-purine | acid | | | | |
| 1bm | 2-(3- | 3- | 3-amino-N-tert- | 4 | 2.25 | 373 |
| | aminosulfonylphenyl)amino- | thiophenboronic | butylbenzenesulfonamide | | | |
| | 6-(thien-3-yl)-9H-purine | acid | | | | |
| 1bn | 6-(2,5-difluorophenyl)-2-(3- | 2,5- | (3- | 4 | 3.22 | 415 |
| | phenylaminophenyl)amino- | difluorophenyl | phenylaminophenyl)amine | | | |
| | 9H-purine | boronic | | | | |
| | | acid | | | | |
| 1bo | 6-(3- | 3- | (3- | 4 | 3.15 | 457 |
| | methanesulfonylphenyl)-2- | methanesulfonylphenylboronic | phenylaminophenyl)amine | | | |
| | (3- | acid | | | | |
| | phenylaminophenyl)amino- | | | | | |
| | 9H-purine | | | | | |
| 1bp | 6-[4-(N- | 6-[4-(N- | (3- | 4 | 2.85 | 436 |
| | acetyl)aminophenyl]-2-(3- | acetyl)aminophenyl]boronic | phenylaminophenyl)amine | | | |
| | phenylaminophenyl)amino- | acid | | | | |
| | 9H-purine | | | | | |
| 1bq | 2-(3- | 3- | 3-amino-N-tert- | 4 | 2.12 | 445 |
| | aminosulfonylphenyl)amino- | methanesulfonylphenylboronic | butylbenzenesulfonamide | | | |
| | 6-(3- | acid | | | | |
| | methanesulfonylphenyl)- | | | | | |
| | 9H-purine | | | | | |
| 1br | 2-(3- | Reference | 3-amino-N-tert- | 1 | 5.44 | 466 |
| | Aminosufonylphenyl)amino- | example 9 | butylbenzenesulfonamide | | | |
| | 6-{4-[(S)-3- | | | | | |
| | hydroxypiperidin-1- | | | | | |
| | yl]phenyl}-9H-purine | | | | | |
| 1bs | 2-[3- | 4- | 3- | 5 | 1.29 | 424 |
| | (aminosulfonyl)phenyl]amino- | (methylaminocarbonyl)phenyl | aminobenzenesulfonamide (1) | | | |
| | 6-(4- | boronic | | | | |
| | (methylaminocarbonyl)phenyl)- | acid | | | | |
| | 9H-purine | | | | | |
| 1bt | 2-[3- | 4- | N-(3- | 5 | 1.48 | 428 |
| | (acetylamino)phenyl]amino- | (cyclopropylaminocarbonyl)phenyl | aminophenyl)acetamide (1) | | | |
| | 6-(4- | boronic | | | | |
| | (cyclopropylaminocarbonyl)phenyl)- | acid | | | | |
| | 9H-purine | | | | | |
| 1bu | 2-[3- | 3- | N-(3- | 5 | 1.31 | 388 |
| | (acetylamino)phenyl]amino- | carbamoylphenylboronic | aminophenyl)acetamide (1) | | | |
| | 6-(3-carbamoyl)phenyl- | acid | | | | |
| | 9H-purine | | | | | |
| 1bv | 2-[3- | 3- | 3- | 5 | 1.26 | 410 |
| | (aminosulfonyl)phenyl]amino- | carbamoylphenylboronic | aminobenzenesulfonamide (1) | | | |
| | 6-(3-carbamoyl)phenyl- | acid | | | | |
| | 9H-purine | | | | | |
| 1bw | 6-(3-carbamoyl)phenyl-2- | 3- | [4-(4- | 5 | 1.47 | 416 |
| | [4-(4- | carbamoylphenylboronic | morpholino)phenyl]amine (1) | | | |
| | morpholino)phenyl]amino- | acid | | | | |
| | 9H-purine | | | | | |
| 1bx | 6-(3-carbamoyl)phenyl-2- | 3- | 4-(2- | 5 | 1.33 | 375 |
| | [4-(2- | carbamoylphenylboronic | hydroxyethyl)phenylamine (1) | | | |
| | hydroxyethyl)phenyl]amino- | acid | | | | |
| | 9H-purine | | | | | |
| 1by | 6-(3-carbamoyl)phenyl-2- | 3- | Reference | 5 | 1.23 | 454 |
| | [4-(2- | carbamoylphenylboronic | Example 7 (1) | | | |
| | hydroxyethyl)sulfonylphenyl]amino- | acid | | | | |
| | 9H-purine | | | | | |
| 1bz | 2-[4-(2- | 3- | Reference | 5 | 1.61 | 417 |
| | hydroxyethyl)sulfonylphenyl]amino- | thiopheneboronic | Example 7 (1) | | | |
| | 6-(thien-3-yl)-9H- | acid | | | | |
| | purine | | | | | |
| 1ca | 6-(3-carbamoyl)phenyl-2- | 3- | [4-(4- | 5 | 1.37 | 429 |
| | [4-(4-methylpiperazin-1- | carbamoylphenylboronic | methylpiperazin- | | | |
| | yl)phenyl]amino-9H-purine | acid | 1- | | | |
| | | | yl)phenyl]amine (1) | | | |
| 1cb | 6-(4-acetylamino)phenyl-2- | 4- | 2-methyl-5- | 4 | 2.05 | 438 |
| | (4-methyl-3- | (acetylamino)phenyl | aminobenzenesulfonamide | | | |
| | aminosulfonylphenyl)amino- | boronic | | | | |
| | 9H-purine | acid | | | | |
| 1cc | 6-(4-acetylamino)phenyl-2- | 4- | 5-amino-2- | 4 | 1.87 | 454 |
| | (4-methoxy-3- | (acetylamino)phenyl | methoxybenzene | | | |
| | aminosulfonylphenyl)amino- | boronic | sulfonamide | | | |
| | 9H-purine | acid | | | | |
| 1cd | 6-(4-acetylamino)phenyl-2- | 4- | 3-amino-N- | 4 | 2.15 | 438 |
| | (3- | (acetylamino)phenyl | methylbenzene | | | |
| | methylaminosulfonylphenyl)amino- | boronic | sulfonamide | | | |
| | 9H-purine | acid | | | | |
| 1ce | 6-(3-carbamoyl)phenyl-2- | 3- | 3-(pyrrolidin-1- | 5 | 1.35 | 451 |
| | [3-(pyrrolidin-1- | carbamoylphenylboronic | ylmethyl)aniline (1) | | | |
| | ylmethyl)phenyl]amino-9H- | acid | | | | |
| | purine | | | | | |
| 1cf | 6-(3- | 3- | 5-amino-2- | 4 | 2.12 | 475 |
| | methanesulfonyl)phenyl-2- | (methanesulfonyl)phenylboronic | methoxybenzenesulfonamide (1) | | | |
| | (4-methoxy-3- | acid | | | | |
| | aminosulfonylbenzene)amino- | | | | | |
| | 9H-purine | | | | | |
| 1cg | 2-(3- | 4-N- | 3- | 5 | 1.74 | 410 |
| | Aminosufonylphenyl)amino- | (dimethylamino)phenyl | aminobenzenesulfonamide (1) | | | |
| | 6-(4- | boronic | | | | |
| | dimethylaminophenyl)-9H- | acid | | | | |
| | purine | | | | | |
| 1ch | 2-[3- | 3- | N-(3- | 5 | 1.90 | 391 |
| | (acetylamino)phenyl]amino | (methylsulfanyl)phenylboronic | aminophenyl)acetamide | | | |
| | 6-(3- | acid | | | | |
| | methylsulfanylphenyl)-9H- | | | | | |
| | purine | | | | | |
| 1ci | 2-[4-(3-(R)- | 3- | Reference | 1 | 7.16 | 433 |
| | hydroxypiperidin-1- | (methylsulfanyl)phenylboronic | Example 8e (1) | | | |
| | yl)phenyl]amino-6-(3- | acid | | | | |
| | methylsulfanylphenyl)-9H- | | | | | |
| | purine | | | | | |
| 1cj | 2-(3- | 4- | 3- | 1 | 6.72 | 413 |
| | aminosulfonyl)phenylamino- | (methylsulfanyl)phenylboronic | aminobenzenesulfonamide (1) | | | |
| | 6-(4- | acid | | | | |
| | methylsulfanylphenyl)-9H- | | | | | |
| | purine | | | | | |
| 1ck | 2-[3- | 3- | N-(3- | 5 | 1.45 | 423 |
| | (acetylamino)phenyl]amino | (Methanesulfonyl)phenylboronic | aminophenyl)acetamide (1) | | | |
| | 6-(4- | acid | | | | |
| | methanesulfonyl)phenyl- | | | | | |
| | 9H-purine | | | | | |
| 1cl | 2-[(4-piperidin-3- | 3- | 4-(N-tert- | 4 | 1.72 | 377 |
| | yl)phenyl]amino-6-(thien-3- | thiopheneboronic | butoxycarbonylpiperidin- | | | |
| | yl)-9H-purine | acid | 3- | | | |
| | | | yl)phenylamine | | | |
| 1cm | 2-(4- | 3- | 2-(4- | 4 | 2.37 | 338 |
| | hydroxyethylphenyl)amino- | thiopheneboronic | aminophenyl)ethanol | | | |
| | 6-(thien-3-yl)-9H-purine | acid | | | | |
| 1cn | 6-[4-(N- | 4-(N- | 3,4- | 4 | 2.15 | 405 |
| | acetyl)aminophenyl]-2- | acetyl)aminophenylboronic | dimethoxyphenylamine | | | |
| | (3,4- | acid | | | | |
| | dimethoxyphenyl)amino- | | | | | |
| | 9H-purine | | | | | |
| 1co | 2-[3- | 3- | N-(3- | 4 | 2.23 | 423 |
| | (acetylamino)phenyl]amino- | (methanesulfonyl)phenylboronic | aminophenyl)acetamide | | | |
| | (3- | acid | | | | |
| | methanesulfonyl)phenyl- | | | | | |
| | 9H-purine | | | | | |
| 1cp | 2-(3-hydroxyphenyl)amino- | 3- | 3- | 4 | 2.27 | 382 |
| | 6-(3- | (methanesulfonyl)phenylboronic | hydroxyaniline | | | |
| | methanesulfonyl)phenyl- | acid | | | | |
| | 9H-purine | | | | | |
| 1cq | 2-[4-(1,1- | 3- | [4-(1,1- | 4 | 2.33 | 499 |
| | dioxothiomorpholin-4- | (methanesulfonyl)phenylboronic | dioxothiomorpholin- | | | |
| | yl)phenyl]amino-6-(3- | acid | 4- | | | |
| | methanesulfonyl)phenyl- | | yl)phenyl]amine | | | |
| | 9H-purine | | | | | |
| 1cr | 2-(3,4- | 3- | 3,4- | 4 | 2.48 | 426 |
| | dimethoxyphenyl)amino-6- | (methanesulfonyl)phenylboronic | dimethoxyphenylamine | | | |
| | (3- | acid | | | | |
| | methanesulfonyl)phenyl- | | | | | |
| | 9H-purine | | | | | |
| 1cs | 6-[4-(N- | 4-(N- | [4-(1,1- | 4 | 2.07 | 478 |
| | acetyl)amino]phenyl-2-[4- | acetyl)aminophenylboronic | dioxothiomorpholin- | | | |
| | (1,1-dioxothiomorpholin-4- | acid | 4- | | | |
| | yl) phenyl]amino-9H-purine | | yl)phenyl]amine | | | |
| 1ct | 2-[(4- | 3- | 2-(4- | 2 | 2.22 | 410 |
| | hydroxyethyl)phenyl)]amino- | (methanesulfonyl)phenylboronic | aminophenyl)ethanol | | | |
| | 6-(3- | acid | | | | |
| | methanesulfonyl)phenyl- | | | | | |
| | 9H-purine | | | | | |
| 1cu | 6-(3- | 3- | 3- | 4 | 2.93 | 411 |
| | methanesulfonyl)phenyl-2- | (methanesulfonyl)phenylboronic | nitrophenylamine | | | |
| | (3-nitrophenyl)amino-9H- | acid | | | | |
| | purine | | | | | |
| 1cv | 6-[4- | 4- | [4-(4- | 5 | 2.00 | 416 |
| | (dimethylamino)]phenyl-2- | (dimethylamino)phenylboronic | morpholino)phenyl]amine | | | |
| | [4-(4- | acid | | | | |
| | morpholino)phenyl]amino- | | | | | |
| | 9H-purine | | | | | |
| 1cw | 2-[(3- | 3- | Ethyl-3- | 4 | 3.02 | 438 |
| | ethoxycarbonyl)phenyl]amino- | (methanesulfonyl)phenylboronic | aminobenzoate | | | |
| | 6-(3- | acid | | | | |
| | methanesulfonyl)phenyl- | | | | | |
| | 9H-purine | | | | | |
| 1cx | 6-(4-N- | N-tert- | [4-(4- | 4 | 1.90 | 402 |
| | methylamio)phenyl-2-[4- | butoxycarbonyl- | morpholino)phenyl]amine | | | |
| | (4- | N- | | | | |
| | morpholino)phenyl]amino- | methylamine- | | | | |
| | 9H-purine | 4- | | | | |
| | | (4,4,5,5- | | | | |
| | | tetramethyl- | | | | |
| | | 1,3,2- | | | | |
| | | dioxoborolan- | | | | |
| | | 2- | | | | |
| | | yl)aniline | | | | |
| 1cy | 2-(3- | 4- | 3- | 5 | 1.13 | 410 |
| | aminosufonylphenyl)amino- | carbamoylphenylboronic | aminobenzenesulfonamide (1) | | | |
| | 6-(4-carbamoylphenyl) | acid | | | | |
| | 9H-purine | | | | | |
| 1cz | 2-[(3-fluoro-4- | 3- | 3-fluoro-4- | 4 | 2.77 | 414 |
| | methoxy)phenyl]amino-6- | (methanesulfonyl)phenylboronic | methoxyaniline (1) | | | |
| | (3- | acid | | | | |
| | methanesulfonyl)phenyl- | | | | | |
| | 9H-purine | | | | | |
| 1da | 6-(3- | 3- | 4-[4-tert- | 4 | 1.63 | 449 |
| | methanesulfonyl)phenyl-2- | (methanesulfonyl)phenylboronic | butoxycarbonylpiperazin- | | | |
| | [(4-piperazin-1- | acid | 1- | | | |
| | yl)phenyl]amino-9H-purine | | yl]phenylamine | | | |
| 1db | 6-[4-(N- | 4-(N- | 3-(pyrrolidin-1- | 4 | 1.53 | 428 |
| | acetyl)amino]phenyl-2-[(3- | acetyl)aminophenylboronic | ylmethyl)aniline (1) | | | |
| | pyrrolidin-1-ylmethyl)phenyl]amino- | acid | | | | |
| | 9H-purine | | | | | |
| 1dc | 6-(3- | 3- | 3-(pyrrolidin-1- | 4 | 1.63 | 449 |
| | methanesulfonyl)phenyl-2- | (methanesulfonyl)phenylboronic | ylmethyl)aniline (1) | | | |
| | [(3-pyrrolidin-1- | acid | | | | |
| | ylmethyl)phenyl]amino-9H- | | | | | |
| | purine | | | | | |
| 1dd | 6-(3- | 3- | N-tert- | 4 | 1.42 | 435 |
| | methanesulfonyl)phenyl-2- | (methanesulfonyl)phenylboronic | butoxycarbonyl- | | | |
| | [3-(2- | acid | 2-(3- | | | |
| | pyrrolidinyl)phenyl]amino- | | aminophenyl)pyrrolidine (1) | | | |
| | 9H-purine | | | | | |
| 1de | 2-(3- | Reference | 3- | 4 | 2.55 | 484 |
| | aminosufonylphenyl)amino- | Example | aminobenzenesulfonamide (1) | | | |
| | 6-[1-(methanesulfonyl)- | 14 | | | | |
| | 1H-indol-5-yl]-9H-purine | | | | | |

(1) In section b, wo equivalents of K₂CO₃ were used instead of NaOtBu, 0.1 equivalents of X-Phos were used instead of BINAP, and tert-butanol was used instead of toluene.

### Example 2

### 6-[6-(4-Acetyl[1,4]diazepan-1-yl)pyridin-3-yl]-2-[4-(4-morpholino)phenyl]amino-9H-purine

### a) 6-[6-(4-Acetyl[1,4]diazepan-1-yl)pyridin-3-yl]-2-chloro-9-(tetrahydropyran-2-yl)-9H-purine

To a solution of reference example 2 (0.30 g, 0.89 mmol) and DIEA (0.47 mL, 2.69 mmol) in n-BuOH (25 mL), N-acetylhomopiperazine (0.51 g, 3.59 mmol) was added. The mixture was heated for 18 h at 120° C., it was allowed to cool, and was concentrated to dryness. The crude product obtained was chromatographed over silica gel using hexane/EtOAc mixtures of increasing polarity as eluent, to afford 0.26 g of the desired compound (63% yield).

LC-MS (method 1): t_{R}=7.24 min; m/z=456 (MH⁺).

### b) 6-[6-(4-Acetyl[1,4]diazepan-1-yl)pyridin-3-yl]-2-[4-(4-morpholino)phenyl]amino-9-(tetrahydropyran-2-yl)-9H-purine

Following a similar procedure to that described in example 1 section b, but using the compound obtained in the previous section, the desired compound was obtained (76% yield).

LC-MS (method 2): t_{R}=2.55 min; m/z=598 (MH⁺).

### c) Title Compound

Following a similar procedure to that described in example 1 section c, but using the compound obtained in the previous section, the title compound of the example was obtained (53% yield).

LC-MS (method 1): t_{R}=4.34 min; m/z=514 (MH⁺).

Following a similar procedure to that described in example 2, but using in each case the corresponding starting materials, these compounds were obtained:

| | | reagent for step | reagent for | HPLC | t_{R} | |
|---|---|---|---|---|---|---|
| Example | Compound name | a) | step b) | method | (min) | m/z |
| 2a | 6-[6-(3-hydroxypiperidin-1- | 3- | (3- | 1 | 7.45 | 479 |
| | yl)pyridin-3-yl]-2-(3- | hydroxypiperidine | phenylaminophenyl)amine | | | |
| | phenylaminophenyl)amino- | hydrochloride | | | | |
| | 9H-purine | | | | | |
| 2b | 6-[6-(4- | N- | (3- | 1 | 7.07 | 520 |
| | acetyl[1,4]diazepan-1- | acetylhomopiperazine | phenylaminophenyl)amine | | | |
| | yl)pyridin-3-yl]-2-(3- | | | | | |
| | phenylaminophenyl)amino- | | | | | |
| | 9H-purine | | | | | |
| 2c | 6-[6-(4- | N- | Reference | 1 | 5.35 | 528 |
| | acetyl[1,4]diazepan-1- | acetylhomopiperazine | Example 8 | | | |
| | yl)pyridin-3-yl]-2-[4-(3- | | | | | |
| | hydroxypiperidin-1- | | | | | |
| | yl)phenyl]amino-9H-purine | | | | | |
| 2d | 6-[6-(4- | N- | (3- | 1 | 6.69 | 473 |
| | acetyl[1,4]diazepan-1- | acetylhomopiperazine | ethoxyphenyl)amine | | | |
| | yl)pyridin-3-yl]-2-(3- | | | | | |
| | ethoxyphenyl)amino-9H- | | | | | |
| | purine | | | | | |
| 2e | 2-[4-(3-hydroxypiperidin-1- | 3- | Reference | 3 | 3.58 | 487 |
| | yl)phenyl]amino-6-[6-(3- | hydroxypiperidine | Example 8 | | | |
| | hydroxypiperidin-1- | hydrochloride | | | | |
| | yl)pyridin-3-yl]-9H-purine | | | | | |
| 2f | 6-[6-(3-hydroxypiperidin-1- | 3- | [4-(4- | 3 | 4.20 | 473 |
| | yl)pyridin-3-yl]-2-[4-(4- | hydroxypiperidine | morpholino)phenyl]amine | | | |
| | morpholino)phenyl]amino- | hydrochloride | | | | |
| | 9H-purine | | | | | |
| 2g | 6-[6-(3-aminopiperidin-1- | 3-N-tert- | [4-(4- | 3 | 3.90 | 472 |
| | yl)pyridin-3-yl]-2-[4-(4- | butoxycarbonylaminopiperidine | morpholino)phenyl]amine | | | |
| | morpholino)phenyl]amino- | | | | | |
| | 9H-purine | | | | | |
| 2h | 6-[6-(3-aminopiperidin-1- | 3-N-tert- | (3- | 1 | 6.52 | 431 |
| | yl)pyridin-3-yl]-2-(3- | butoxycarbonylaminopiperidine | ethoxyphenyl)amine | | | |
| | ethoxyphenyl)amino-9H- | | | | | |
| | purine | | | | | |
| 2i | 6-[6-(3-aminopiperidin-1- | 3-N-tert- | (3- | 1 | 7.13 | 478 |
| | yl)pyridin-3-yl]-2-(3- | butoxycarbonylaminopiperidine | phenylaminophenyl)amine | | | |
| | phenylaminophenyl)amino- | | | | | |
| | 9H-purine | | | | | |
| 2j | 6-[6-(3-aminopiperidin-1- | 3-N-tert- | Reference | 1 | 5.16 | 486 |
| | yl)pyridin-3-yl]-2-[4-(3- | butoxycarbonylaminopiperidine | Example 8 | | | |
| | hydroxypiperidin-1- | | | | | |
| | yl)phenyl]amino-9H-purine | | | | | |
| 2k | 6-[6-(4- | N- | 3-amino-N- | 1 | 4.93 | 508 |
| | acetyl[1,4]diazepan-1- | acetylhomopiperazine | tert- | | | |
| | yl)pyridin-3-yl]-2-(3- | | butylbenzenesulfonamide | | | |
| | aminosulfonylphenyl)amino- | | | | | |
| | 9H-purine | | | | | |
| 2l | 2-(3-ethoxyphenyl)amino- | N-tert- | (3- | 1 | 6.18 | 417 |
| | 6-[6-(piperazin-1- | butoxycarbonylpiperazine | ethoxyphenyl)amine | | | |
| | yl)pyridin-3-yl]-9H-purine | | | | | |
| 2m | 2-(3- | N-tert- | 3-amino-N- | 1 | 4.27 | 452 |
| | aminosulfonylphenyl)amino- | butoxycarbonylpiperazine | tert- | | | |
| | 6-[6-(piperazin-1- | | butylbenzenesulfonamide | | | |
| | yl)pyridin-3-yl]-9H-purine | | | | | |
| 2n | 2-(3- | N-tert- | (3- | 1 | 6.81 | 464 |
| | phenylaminophenyl)amino- | butoxycarbonylpiperazine | phenylaminophenyl)amine | | | |
| | 6-[6-(piperazin-1- | | | | | |
| | yl)pyridin-3-yl]-9H-purine | | | | | |
| 2o | 6-[6-(4-methylpiperazin-1- | N- | [4-(4- | 4 | 1.37 | 472 |
| | yl)pyridin-3-yl]-2-[4-(4- | methylpiperazine | morpholino)phenyl]amine | | | |
| | morpholino)phenyl]amino- | | | | | |
| | 9H-purine | | | | | |
| 2p | 6-(6- | cyclohexylamine | [4-(4- | 4 | 1.90 | 471 |
| | cyclohexylaminopyridin-3- | | morpholino)phenyl]amine | | | |
| | yl)-2-[4-(4- | | | | | |
| | morpholino)phenyl]amino- | | | | | |
| | 9H-purine | | | | | |
| 2q | 2-[4-(4- | N-tert- | [4-(4- | 4 | 1.35 | 458 |
| | morpholino)phenyl]amino- | butoxycarbonylpiperazine | morpholino)phenyl]amine | | | |
| | 6-[6-(piperazin-1- | | | | | |
| | yl)pyridin-3-yl]-9H-purine | | | | | |
| 2r | 2-(3- | N- | 3- | 5 | 1.90 | 439 |
| | aminosufonylphenyl)amino- | methylpropylamine | aminobenzenesulfonamide (1) | | | |
| | 6-[6-(N- | | | | | |
| | methylpropylamino)pyridin- | | | | | |
| | 3-yl]-9H-purine | | | | | |
| 2s | 2-(3- | N- | N-(3- | 5 | 1.89 | 417 |
| | acetylaminophenyl)amino- | methylpropylamine | aminophenyl)acetamide | | | |
| | 6-[6-(N- | | | | | |
| | methylpropylamino)pyridin- | | | | | |
| | 3-yl]-9H-purine | | | | | |
| 2t | 2-(3- | 4- | N-(3- | 4 | 1.5 | 445 |
| | acetylaminophenyl)amino- | hydroxypiperidine (2) | aminophenyl)acetamide | | | |
| | 6-[6-(4-hydroxypiperidin-1- | | | | | |
| | yl)pyridin-3-yl]-9H-purine | | | | | |
| 2u | 2-(3-acetylphenyl)amino- | 4- | 3′- | 4 | 1.75 | 430 |
| | 6-[6-(4-hydroxypiperidin-1- | hydroxypiperidine (2) | aminoacetophenone (1) | | | |
| | yl)pyridin-3-yl]-9H-purine | | | | | |
| 2v | 2-(3- | 4- | 3- | 4 | 1.47 | 467 |
| | aminosufonylphenyl)amino- | hydroxypiperidine (2) | aminobenzenesulfonamide (1) | | | |
| | 6-[6-(4-hydroxypiperidin- | | | | | |
| | 1-yl)pyridin-3-yl]-9H- | | | | | |
| | purine | | | | | |
| 2w | 6-[6-(3-hydroxypiperidin-1- | 3- | Reference | 4 | 167 | 454 |
| | yl)pyridin-3-yl]-2-[4- | hydroxypiperidine | Example 10 (3) | | | |
| | (pyrazol-3- | hydrochloride | | | | |
| | yl)phenyl]amino-9H-purine | | | | | |

(1) Two equivalents of K₂CO₃ were used instead of NaOtBu, 0.1 equivalents of X-Phos were used instead of BINAP, and tert-buthanol was used instead of toluene.
(2) Using ethanol, instead of n-butanol.
(3) An additional deprotection step was necessary: over a solution of the product obtained in section c (1 eq) in THF (10 mL) 4.8 eq. of a 1M solution of TBAF were added. The reaction was refluxed for 5 h and the mixture thus obtained was partitioned between H₂O and dichloromethane. The organic phase was dried over Na₂SO₄ and concentrated to dryness. The crude product thus obtained was chromatographed over silica gelusing CHCl₃:MeOH:NH₃ mixtures of increasing polarity as eluent, to afford the desired compound.

### Example 3

### 6-[6-(4-Acetylpiperazin-1-yl)pyridin-3-yl]-2-(3-phenylaminophenyl)amino-9H-purine

To a solution of example 2n (24.90 mg, 0.05 mmol) in CH₂Cl₂ (0.50 mL) and TEA (11.22 μL, 0.08 mmol) under Ar-atmosphere, acetic anhydride (5.58 μL, 0.06 mmol) was added at 0° C. The resulting mixture was stirred at room temperature for 1 h. Water was added and the phases were separated. The aqueous layer was extracted twice with CH₂Cl₂. The combined organic phases were dried over Na₂SO₄ and concentrated to dryness. The crude product obtained was chromatographed over silica gel using 10% EtOAc/MeOH as eluent, to afford 16.4 mg of the title compound of the example (60% yield).

LC-MS (method 1): t_{R}=6.82 min; m/z=506 (MH⁺).

Following a similar procedure to that described in example 3, but using in each case the corresponding starting materials, these compounds were obtained:

| | | Starting | HPLC | | |
|---|---|---|---|---|---|
| Example | Compound name | material | method | t_{R} (min) | m/z |
| 3a | 6-[6-(4-acetylpiperazin-1-yl)pyridin-3-yl]-2- | Example | 1 | 6.40 | 459 |
| | (3-ethoxyphenyl)amino-9H-purine | 2l | | | |
| 3b | 6-[6-(4-acetylpiperazin-1-yl)pyridin-3-yl]-2- | Example | 1 | 4.75 | 494 |
| | (3-aminosulfonylphenyl)amino-9H-purine | 2m | | | |

### Example 4

### 6-[6-(4-Aminopiperidin-1-yl)pyridin-3-yl]-2-[3-(4-morpholino)phenyl]amino-9H-purine

### a) 6-Hydroxy-2-[3-(4-morpholino)phenyl]amino-9H-purine

To a solution of 2-bromohypoxanthine (0.50 g, 2.32 mmol) in 2-methoxyethanol (6 mL) and H₂O (5 mL), [3-(4-morpholino)phenyl]amine (0.86 g, 4.87 mmol) was added. The mixture was heated for 18 h at 120° C. It was cooled and H₂O (20 mL) was added. A white solid precipitated which was separated by filtration and dried under vacuum. 0.71 g of the desired compound was obtained (99% yield).

LC-MS (method 1): t_{R}=3.62 min; m/z=313 (MH⁺).

### b) 6-Chloro-2-[3-(4-morpholino)phenyl]amino-9H-purine

In a flask were mixed, under Ar-atmosphere, the compound obtained in the previous section (0.71 g, 2.30 mmol), POCl₃ (5.5 mL) and N,N-dimethylaniline (0.70 mL). The mixture was refluxed for 1 h. It was cooled and, at 0° C., the mixture was poured over H₂O. Sodium acetate was added until pH=4. A white solid precipitated which was separated by filtration and dried in a vacuum heater. 0.46 g of the desired compound was obtained (61% yield).

LC-MS (method 2): t_{R}=2.13 min; m/z=331 (MH⁺).

### c) 6-Chloro-2-[3-(4-morpholino)phenyl]amino-9-(tetrahydropyran-2-yl)-9H-purine

Following a similar procedure to that described in reference example 1, but using the compound obtained in the previous section, 0.41 g of the desired compound was obtained (71% yield).

LC-MS (method 1): t_{R}=7.34 min; m/z=415 (MH⁺).

### d) 6-[6-[4-(tert-Butoxycarbonyl)aminopiperidin-1-yl]pyridin-3-yl]-2-[3-(4-morpholino)phenyl]amino-9-(tetrahydropyran-2-yl)-9H-purine

Following a similar procedure to that described in example 1 section a, but using the compound obtained in the previous section, the desired compound was obtained (50% yield).

LC-MS (method 1): t_{R}=7.67 min; m/z=656 (MH⁺).

### e) Title Compound

Following a similar procedure to that described in example 1 section c, but using the compound obtained in the previous section, the title compound of the example was obtained (8% yield).

LC-MS (method 1): t_{R}=5.52 min; m/z=472 (MH⁺).

Following a similar procedure to that described in example 4, but using in each case the corresponding starting materials, these compounds were obtained:

| | | reagent for | reagent for step | HPLC | t_{R} | |
|---|---|---|---|---|---|---|
| Example | Compound name | step a) | b) | method | (min) | m/z |
| 4a | 2-(3-ethoxyphenyl)amino-6- | (3- | 3-pyridylboronic | 1 | 6.48 | 333 |
| | (3-pyridyl)-9H-purine | methoxyphenyl)amine | acid | | | |
| 4b | 6-[6-(4-aminopiperidin-1- | aniline | Reference | 1 | 5.51 | 387 |
| | yl)pyridin-3-yl]-2- | | Example 3 | | | |
| | phenylamino-9H-purine | | | | | |
| 4c | 2-(3-methoxyphenyl)amino- | (3- | 4- | 4 | 3.60 | 386 |
| | 6-(4-trifluoromethylphenyl)- | methoxyphenyl)amine | trifluoromethylphenylboronic | | | |
| | 9H-purine | | acid | | | |
| 4d | 6-(3-methoxyphenyl)-2-(3- | (3- | 3- | 4 | 3.05 | 348 |
| | methoxyphenyl)amino-9H- | methoxyphenyl)amine | methoxyphenylboronic | | | |
| | purine | | acid | | | |
| 4e | 6-[4-(piperazin-1- | aniline | Reference | 1 | 6.43 | 436 |
| | sulfonyl)phenyl]-2- | | Example 6 | | | |
| | phenylamino-9H-purine | | | | | |
| 4f | 6-(3-methoxyphenyl)-2-[4- | [4-(4- | 3-methoxyphenylboronic | 4 | 2.32 | 403 |
| | (4- | morpholino)phenyl]amine | acid | | | |
| | morpholino)phenyl]amino- | | | | | |
| | 9H-purine | | | | | |
| 4g | 6-(5-bromo-2-fluorophenyl)- | [4-(4- | 5-bromo-2- | 1 | 7.15 | 471 |
| | 2-[4-(4- | morpholino)phenyl]amine | fluorophenylboronic | | | |
| | morpholino)phenyl]amino- | | acid | | | |
| | 9H-purine | | | | | |
| 4h | 6-(1H-indol-5-yl)-2-[4-(4- | [4-(4- | 5-indolylboronic | 4 | 2.00 | 412 |
| | morpholino)phenyl]amino- | morpholino)phenyl]amine | acid | | | |
| | 9H-purine | | | | | |
| 4i | 6-(3-chlorophenyl)-2-[4-(4- | [4-(4- | 3- | 4 | 2.78 | 407 |
| | morpholino)phenyl]amino- | morpholino)phenyl]amine | chlorophenylboronic | | | |
| | 9H-purine | | acid | | | |
| 4j | 6-(3-acetylphenyl)-2-[4-(4- | [4-(4- | 3- | 4 | 2.25 | 415 |
| | morpholino)phenyl]amino- | morpholino)phenyl]amine | acetylphenylboronic | | | |
| | 9H-purine | | acid | | | |
| 4k | 6-(3-cyanophenyl)-2-[4-(4- | [4-(4- | 3- | 4 | 2.38 | 398 |
| | morpholino)phenyl]amino- | morpholino)phenyl]amine | cyanophenylboronic | | | |
| | 9H-purine | | acid | | | |
| 4l | 2-[4-(4- | [4-(4- | 3- | 4 | 2.18 | 379 |
| | morpholino)phenyl]amino- | morpholino)phenyl]amine | thiophenboronic | | | |
| | 6-(thien-3-yl)-9H-purine | | acid | | | |
| 4m | 6-[4-(N- | [4-(4- | 6-[4-(N- | 4 | 1.82 | 430 |
| | acetyl)aminophenyl]-2-[4- | morpholino)phenyl]amine | acetyl)aminophenyl]boronic | | | |
| | (4- | | acid | | | |
| | morpholino)phenyl]amino- | | | | | |
| | 9H-purine | | | | | |
| 4n | 6-(3-chloro-4-fluorophenyl)- | [4-(4- | 3-chloro-4- | 4 | 2.93 | 425 |
| | 2-[4-(4- | morpholino)phenyl]amine | fluorophenylboronic | | | |
| | morpholino)phenyl]amino- | | acid | | | |
| | 9H-purine | | | | | |
| 4o | 6-(5-fluoro-2- | [4-(4- | 5-fluoro-2- | 4 | 2.02 | 421 |
| | methoxyphenyl)-2-[4-(4- | morpholino)phenyl]amine | methoxyphenylboronic | | | |
| | morpholino)phenyl]amino- | | acid | | | |
| | 9H-purine | | | | | |
| 4p | 6-(2,5-difluorophenyl)-2-[4- | [4-(4- | 2,5- | 4 | 2.10 | 409 |
| | (4- | morpholino)phenyl]amine | difluorophenylboronic | | | |
| | morpholino)phenyl]amino- | | acid | | | |
| | 9H-purine | | | | | |
| 4q | 6-(3-fluoro-4- | [4-(4- | 3-fluoro-4- | 4 | 2.47 | 421 |
| | methoxyphenyl)-2-[4-(4- | morpholino)phenyl]amine | methoxyphenylboronic | | | |
| | morpholino)phenyl]amino- | | acid | | | |
| | 9H-purine | | | | | |
| 4r | 6-[6-(4-aminopiperidin-1- | (3- | Reference | 1 | 5.99 | 431 |
| | yl)pyridin-3-yl]-2-(3- | ethoxyphenyl)amine | Example 3 | | | |
| | ethoxyphenyl)amino-9H- | | | | | |
| | purine | | | | | |
| 4s | 6-(3- | [4-(4- | 3- | 4 | 2.02 | 451 |
| | methanesulfonylphenyl)-2- | morpholino)phenyl]amine | methanesulfonylphenylboronic | | | |
| | [4-(4- | | acid | | | |
| | morpholino)phenyl]amino- | | | | | |
| | 9H-purine | | | | | |
| 4t | 6-(2-chloro-5- | [4-(4- | 2-chloro-5- | 4 | 2.27 | 421 |
| | methylphenyl)-2-[4-(4- | morpholino)phenyl]amine | methylphenylboronic | | | |
| | morpholino)phenyl]amino- | | acid | | | |
| | 9H-purine | | | | | |
| 4u | 6-[4-(N- | [4-(4- | 4-(N- | 4 | 2.17 | 458 |
| | isobutyryl)aminophenyl]-2- | morpholino)phenyl]amine | isobutyryl)aminophenylboronic | | | |
| | [4-(4- | | acid | | | |
| | morpholino)phenyl]amino- | | | | | |
| | 9H-purine | | | | | |
| 4v | 6-[4-(N- | [4-(4- | 4-(N- | 4 | 2.47 | 492 |
| | benzoyl)aminophenyl]-2-[4- | morpholino)phenyl]amine | benzoyl)aminophenyl | | | |
| | (4- | | boronic acid | | | |
| | morpholino)phenyl]amino- | | | | | |
| | 9H-purine | | | | | |
| 4w | 6-(3-bromophenyl)-2-[4-(4- | [4-(4- | 3- | 1 | 8.05 | 451 |
| | morpholino)phenyl]amino- | morpholino)phenyl]amine | bromophenylboronic | | | |
| | 9H-purine | | acid | | | |

### Example 5

### 2-(4-Aminosulfonylphenyl)amino-6-[6-(piperidin-4-yl)aminopyridin-3-yl]-9H-purine

### a) 2-(4-tert-Butylaminosulfonylphenyl)amino-6-(6-fluoropyridin-3-yl)-9-(tetrahydropyran-2-yl)-9H-purine

Following a similar procedure to that described in example 1 section b, but using reference example 2 and 4-tert-butylaminosulfonylaniline, the desired compound was obtained (90% yield).

LC-MS (method 1): t_{R}=8.86 min; m/z=526 (MH⁺).

### b) 2-(4-tert-Butylaminosulfonylphenyl)amino-6-[6-[1-(tert-butoxycarbonyl)piperidin-4-yl]aminopyridin-3-yl]-9-(tetrahydropyran-2-yl)-9H-purine

Following a similar procedure to that described in example 2 section a, but using the compound obtained in the previous section and 4-amino-1-tert-butoxycarbonylpiperidine, the desired compound was obtained (86% yield).

LC-MS (method 1): t_{R}=9.81 min; m/z=706 (MH⁺).

### c) Title Compound

Following a similar procedure to that described in example 1 section c, but using the compound obtained in the previous section, the title compound of the example was obtained (31% yield).

LC-MS (method 1): t_{R}=4.28 min; m/z=466 (MH⁺).

Following a similar procedure to that described in example 5, but using in each case the corresponding starting materials, these compounds were obtained:

| | | reagent for | reagent for | HPLC | t_{R} | |
|---|---|---|---|---|---|---|
| Example | Compound name | step a) | step b) | method | (min) | m/z |
| 5a | 2-[4-(4- | [4-(4- | morpholine | 4 | 1.72 | 459 |
| | morpholino)phenyl]amino-6- | morpholino)phenyl]amine | | | | |
| | [6-(morpholin-4-yl)pyridin-3- | | | | | |
| | yl]-9H-purine | | | | | |
| 5b | 2-[4-(1,1- | [4-(1,1- | 3- | 4 | 1.65 | 521 |
| | dioxothiomorpholin-4- | dioxothiomorpholin- | hydroxypiperidine | | | |
| | yl)phenyl]amino-6-[6-(3- | 4- | hydrochloride | | | |
| | hydroxypiperidin-1- | yl)phenyl]amine | | | | |
| | yl)pyridin-3-yl]-9H-purine | | | | | |
| 5c | 2-[4-(4- | [4-(4- | piperidine | 4 | 1.80 | 457 |
| | morpholino)phenyl]amino-6- | morpholino)phenyl]amine | | | | |
| | [6-(piperidin-1-yl)pyridin-3- | | | | | |
| | yl]-9H-purine | | | | | |
| 5d | 6-[6-(3-hydroxypyrrolidin-1- | [4-(4- | 3- | 4 | 1.40 | 459 |
| | yl)pyridin-3-yl]-2-[4-(4- | morpholino)phenyl]amine | hydroxypyrrolidine | | | |
| | morpholino)phenyl]amino- | | | | | |
| | 9H-purine | | | | | |
| 5e | 6-[6-(2- | [4-(4- | 2-aminoethanol | 4 | 1.32 | 433 |
| | hydroxyethyl)aminopyridin- | morpholino)phenyl]amine | | | | |
| | 3-yl]-2-[4-(4- | | | | | |
| | morpholino)phenyl]amino- | | | | | |
| | 9H-purine | | | | | |
| 5f | 6-[6-(3- | [4-(4- | 3- | 4 | 1.36 | 447 |
| | hydroxypropyl)aminopyridin- | morpholino)phenyl]amine | aminopropanol | | | |
| | 3-yl]-2-[4-(4- | | | | | |
| | morpholino)phenyl]amino- | | | | | |
| | 9H-purine | | | | | |
| 5g | 6-[6-(3- | [4-(4- | 3- | 4 | 1.25 | 486 |
| | dimethylaminopyrrolidin-1- | morpholino)phenyl]amine | dimethylaminopyrrolidine | | | |
| | yl)pyridin-3-yl]-2-[4-(4- | | | | | |
| | morpholino)phenyl]amino- | | | | | |
| | 9H-purine | | | | | |
| 5h | 6-[6-(3-hydroxypiperidin-1- | 3- | 3- | 4 | 2.48 | 480 |
| | yl)pyridin-3-yl]-2-(3- | phenoxyaniline | hydroxypiperidine | | | |
| | phenoxyphenyl)amino-9H- | | hydrochloride | | | |
| | purine | | | | | |
| 5i | 2-[4-(3-aminopiperidin-1- | Reference | 3- | 4 | 1.30 | 486 |
| | yl)phenyl]amino-6-[6-(3- | Example 8c | hydroxypiperidine | | | |
| | hydroxypiperidin-1- | | hydrochloride | | | |
| | yl)pyridin-3-yl]-9H-purine | | | | | |
| 5j | 6-[6-(3-aminopyrrolidin-1- | [4-(4- | 3-tert- | 4 | 1.15 | 458 |
| | yl)pyridin-3-yl]-2-[4-(4- | morpholino)phenyl]amine | butoxycarbonyl | | | |
| | morpholino)phenyl]amino- | | aminopyrrolidine | | | |
| | 9H-purine | | | | | |
| 5k | 6-[6-(2- | [4-(4- | ethylendiamine | 4 | 1.14 | 432 |
| | aminoethyl)aminopyridin-3- | morpholino)phenyl]amine | | | | |
| | yl]-2-[4-(4- | | | | | |
| | morpholino)phenyl]amino- | | | | | |
| | 9H-purine | | | | | |
| 5l | 6-[6-(4-hydroxipiperidin-1- | [4-(4- | 4- | 4 | 1.47 | 473 |
| | yl)pyridin-3-yl]-2-[4-(4- | morpholino)phenyl]amine | hydroxypiperidine | | | |
| | morpholino)phenyl]amino- | | hydrochloride | | | |
| | 9H-purine | | | | | |
| 5m | 6-[6-(4- | [4-(4- | 4- | 4 | 1.25 | 486 |
| | aminomethylpiperidin-1- | morpholino)phenyl]amine | aminomethylpiperidine | | | |
| | yl)pyridin-3-yl]-2-[4-(4- | | | | | |
| | morpholino)phenyl]amino- | | | | | |
| | 9H-purine | | | | | |
| 5n | 2-[4-(4- | [4-(4- | 3- | 4 | 1.19 | 472 |
| | morpholino)phenyl]amino-6- | morpholino)phenyl]amine | (aminomethyl)- | | | |
| | [6-(pyrrolidin-3- | | 1-tert- | | | |
| | ylmethyl)aminopyridin-3-yl]- | | butoxycarbonyl | | | |
| | 9H-purine | | pyrrolidine | | | |
| 5o | 6-[6-(3-hydroxypiperidin-1- | 3-methoxy-5- | 3- | 4 | 2.42 | 486 |
| | yl)pyridin-3-yl]-2-(3- | trifluoromethylaniline | hydroxypiperidine | | | |
| | methoxy-5- | | hydrochloride | | | |
| | trifluorophenyl)amino-9H- | | | | | |
| | purine | | | | | |
| 5p | 6-(6-methoxypyridin-3-yl)-2- | [4-(4- | N- | 5 | 1.79 | 404 |
| | [4-(4- | morpholino)phenyl]amine | methylpropylamine (1) | | | |
| | morpholino)phenyl]amino- | | | | | |
| | 9H-purine | | | | | |
| 5q | 2-[3-(2- | 3-(2- | N- | 5 | 2.03 | 404 |
| | hydroxyethyl)phenyl]amino- | hydroxyethyl)phenylamine (2) | methylpropylamine | | | |
| | 6-[6-(N- | | | | | |
| | methylpropylamino)pyridin- | | | | | |
| | 3-yl]-9H-purine | | | | | |
| 5r | 2-(3- | 3-amino-N- | (S)-3- | 1 | 5.15 | 467 |
| | aminosufonylphenyl)amino- | tert- | hydroxypiperidine | | | |
| | 6-[6-(3-(S)-hydroxypiperidin- | butylbenzene | hydrochloride | | | |
| | 1-yl)pyridin-3-yl]-9H-purine | sulfonamide | | | | |
| 5s | 2-(3- | 3-amino-N- | (R)-3- | 1 | 5.14 | 467 |
| | aminosulfonylphenyl)amino- | tert- | hydroxypiperidine | | | |
| | 6-[6-(3-(R)-hydroxypiperidin- | butylbenzene | hydrochloride | | | |
| | 1-yl)pyridin-3-yl]-9H-purine | sulfonamide | | | | |
| 5t | 6-[6-(3-acetylpiperidin-1- | 3-amino-N- | 3- | 1 | 6.10 | 493 |
| | yl)pyridin-3-yl]-2-(3- | tert- | acetylpiperidine | | | |
| | aminosulfonylphenyl)amino- | butylbenzene | hydrochloride | | | |
| | 9H-purine | sulfonamide | | | | |
| 5u | 6-[(6-N- | [4-(4- | N- | 5 | 2.16 | 445 |
| | methylpropylamino)pyridin- | morpholino)phenyl]amine (3) | methylpropylamine | | | |
| | 3-yl]-2-[4-(4- | | | | | |
| | morpholino)phenyl]amino- | | | | | |
| | 9H-purine | | | | | |
| 5v | 2-[4-(4-methylpiperazin-1- | [4-(4- | N- | 5 | 2.01 | 458 |
| | yl)phenyl]amino-6-[(6-N- | methylpiperazin- | methylpropylamine | | | |
| | methylpropylamino)pyridin- | 1- | | | | |
| | 3-yl]-9H-purine | yl)phenyl]amine | | | | |
| 5w | 2-[4-(3-hydroxypiperidin-1- | Reference | N- | 5 | 2.03 | 460 |
| | yl)phenyl]amino-6-[(6-N- | Example 8 | methylpropylamine | | | |
| | methylpropylamino)pyridin- | | | | | |
| | 3-yl]-9H-purine | | | | | |
| 5x | 6-[(6-N- | N-tert- | N- | 5 | 1.78 | 443 |
| | methylpropylamino)pyridin- | butoxycarbonyl- | methylpropylamine | | | |
| | 3-yl]-2-[4-(piperidin-3- | 3-(4- | | | | |
| | yl)phenyl]amino-9H-purine | aminophenyl)piperidine | | | | |
| 5y | 2-[(4- | 4-(2- | N- | 5 | 1.97 | 404 |
| | hydroxyethyl)phenyl]amino- | hydroxyethyl)phenylamine | methylpropylamine | | | |
| | 6-[6-(N- | | | | | |
| | methypropylamino)pyridin-3- | | | | | |
| | yl]-9H-purine | | | | | |
| 5z | 2-[4-(N-diethylamino)phenyl]amino- | N,N-diethyl- | N- | 5 | 2.7 | 431 |
| | 6-(6-N- | 1,4- | methylpropylamine | | | |
| | methylpropylamino)pyridin- | phenylenediamine | | | | |
| | 3-yl]-9H-purine | | | | | |
| 5aa | 6-(6-N- | 3-(pyrrolidin- | N- | 5 | 2.1 | 443 |
| | methylpropylamino)pyridin- | 1- | methylpropylamine | | | |
| | 3-yl]-2-[(3-pyrrolidinyl-1- | ylmethyl)aniline | | | | |
| | ylmethyl)phenyl]amino-9H- | | | | | |
| | purine | | | | | |
| 5ab | 2-(3- | N-(3- | N- | 5 | 2.21 | 445 |
| | isobutyroylaminophenyl)amino- | aminophenyl)isobutyramide | methylpropylamine | | | |
| | 6-[(6-N- | | | | | |
| | methylpropylamino)pyridin- | | | | | |
| | 3-yl]-9H-purine | | | | | |
| 5ac | 2-[4-(2- | Reference | N- | 5 | 1.84 | 483 |
| | hydroxyethyl)aminosulfonylphenyl]amino- | Example 7 | methylpropylamine | | | |
| | 6-[(6-N- | | | | | |
| | methylpropylamino)pyridin- | | | | | |
| | 3-yl]-9H-purine | | | | | |
| 5ad | 6-[6-(3-(R)-hydroxypiperidin- | [4-(4- | (R)-3- | 5 | 1.65 | 473 |
| | 1-yl)pyridin-3-yl]-2-(4- | morpholino)phenyl]amine | hydroxypiperidine | | | |
| | morpholino)phenyl]amino- | | hydrochloride | | | |
| | 9H-purine | | | | | |
| 5ae | 6-[6-(3-(S)-hydroxypiperidin- | [4-(4- | (S)-3- | 5 | 1.65 | 473 |
| | 1-yl)pyridin-3-yl]-2-(4- | morpholino)phenyl]amine | hydroxypiperidine | | | |
| | morpholino)phenyl]amino- | | hydrochloride | | | |
| | 9H-purine | | | | | |
| 5af | 2-[4-(4-methylpiperazin-1- | [4-(4- | (R)-2- | 5 | 1.99 | 470 |
| | yl)phenyl]amino-6-[6-(2-(R)- | methylpiperazin- | methylpyrrolidine | | | |
| | methylpyrrolidin-1-yl)pyridin- | 1- | | | | |
| | 3-yl]-9H-purine | yl)phenyl]amine | | | | |
| 5ag | 2-[4-(4-methylpiperazin-1- | [4-(4- | (S)-2- | 5 | 1.99 | 470 |
| | yl)phenyl]amino-6-[6-(2-(S)- | methylpiperazin- | methylpyrrolidine | | | |
| | methylpyrrolidin-1-yl)pyridin- | 1- | | | | |
| | 3-yl]-9H-purine | yl)phenyl]amine | | | | |
| 5ah | 2-[4-(4-methylpiperazin-1- | [4-(4- | (S)-2- | 5 | 2.22 | 484 |
| | yl)phenyl]amino-6-[6-(2-(S)- | methylpiperazin- | methylpiperidine | | | |
| | methylpiperidin-1-yl)pyridin- | 1- | | | | |
| | 3-yl]-9H-purine | yl)phenyl]amine | | | | |
| 5ai | 2-[4-(4-methylpiperazin-1- | [4-(4- | (R)-2- | 5 | 2.22 | 484 |
| | yl)phenyl]amino-6-[6-(2-(R)- | methylpiperazin- | methylpiperidine | | | |
| | methylpiperidin-1-yl)pyridin- | 1- | | | | |
| | 3-yl]-9H-purine | yl)phenyl]amine | | | | |
| 5aj | 6-(6-N- | [4-(4- | N,N- | 5 | 1.68 | 430 |
| | dimethylamino)pyridin-3-yl]- | methylpiperazin- | dimethylamine | | | |
| | 2-[4-(4-methylpiperazin-1- | 1- | | | | |
| | yl)phenyl]amino-9H-purine | yl)phenyl]amine | | | | |
| 5ak | 6-[6-(2- | [4-(4- | N-(2- | 5 | 1.72 | 475 |
| | methoxyethyl)methylaminopyridin- | methylpiperazin- | methoxyethyl)methylamine | | | |
| | 3-yl]-2-[4-(4- | 1- | | | | |
| | methylpiperazin-1- | yl)phenyl]amine | | | | |
| | yl)phenyl]amino-9H-purine | | | | | |
| 5al | 2-[(4- | 4-(2- | N-(2- | 5 | 1.68 | 420 |
| | hydroxyethyl)phenyl]amino- | hydroxyethyl)phenylamine | methoxyethyl)methylamine | | | |
| | 6-[6-(2- | | | | | |
| | methoxyethyl)methylaminopyridin- | | | | | |
| | 3-yl]-9H-purine | | | | | |
| 5am | 2-[(3- | 3-(2- | N-(2- | 5 | 1.74 | 421 |
| | hydroxyethyl)phenyl]amino- | hydroxyethyl)phenylamine | methoxyethyl)methylamine | | | |
| | 6-[6-(2- | | | | | |
| | methoxyethyl)methylaminopyridin- | | | | | |
| | 3-yl]-9H-purine | | | | | |
| 5an | 6-[6-(2- | 4-(pyrrolidin- | N-(2- | 5 | 1.60 | 457 |
| | methoxyethyl)methylaminopyridin- | 1- | methoxyethyl)methylamine | | | |
| | 3-yl]-2-[4-(pyrrolidin-1- | ylmethyl)aniline | | | | |
| | ylmethyl)phenyl]amino-9H- | | | | | |
| | purine | | | | | |
| 5ao | 6-[6-(2- | [4-(piperazin- | N-(2- | 5 | 1.42 | 460 |
| | methoxyethyl)methylaminopyridin- | 1- | methoxyethyl)methylamine | | | |
| | 3-yl]-2-[4-(piperazin-1- | yl)phenyl]amine | | | | |
| | yl)phenyl]amino-9H-purine | | | | | |
| 5ap | 2-[4-(2-hydroxy-2- | Reference | N- | 5 | 2.18 | 433 |
| | methylpropyl)]phenylamino- | Example 12 | methylpropylamine | | | |
| | 6-[(6-N- | | | | | |
| | methylpropylamino)pyridin- | | | | | |
| | 3-yl]-9H-purine | | | | | |
| 5aq | 2[4-(cis-3,5- | Reference | N- | 5 | 1.85 | 472 |
| | dimethylpiperazin-1- | Example 8f (3) | methylpropylamine | | | |
| | yl)phenyl]amino-6-[(6-N- | | | | | |
| | methylpropylamino)pyridin- | | | | | |
| | 3-yl]-9H-purine | | | | | |
| 5ar | 2-(N-methyl-3- | Reference | N- | 5 | 2.04 | 431 |
| | acetylaminophenyl)amino-6- | Example 13 (3) | methylpropylamine | | | |
| | [(6-N- | | | | | |
| | methylpropylamino)pyridin- | | | | | |
| | 3-yl]-9H-purine | | | | | |
| 5as | 2-(N-methyl-3- | Reference | N- | 5 | 2.25 | 457 |
| | isobutyroylaminophenyl)amino- | Example 13a (3) | methylpropylamine | | | |
| | 6-[(6-N- | | | | | |
| | methylpropylamino)pyridin- | | | | | |
| | 3-yl]-9H-purine | | | | | |
| 5at | 2-(N-methyl-3- | Reference | N- | 5 | 2.31 | 459 |
| | cyclopropylcarbonylaminophenyl)amino- | Example 13b (3) | methylpropylamine | | | |
| | 6-[(6-N- | | | | | |
| | methylpropylamino)pyridin- | | | | | |
| | 3-yl]-9H-purine | | | | | |

(1) Methanol was added at deprotection step 5c to improve solubility
(2) Cesium carbonate was used instead of NaOtBu.
(3) Two equivalents of K₂CO₃ were used instead of NaOtBu, 0.1 equivalents of X-Phos were used instead of BINAP, and tert-buthanol was used instead of toluene.

### Example 6

### 2-(4-Aminosulfonylphenyl)amino-6-(6-hydroxypyridin-3-yl)-9H-purine

In a flask were mixed, under Ar-atmosphere, the compound obtained in example 5 section a (70 mg, 0.13 mmol) and a mixture of 4M dioxane/HCl_{(g)} (5 mL) with dioxane (4 mL). The reaction mixture was stirred at room temperature overnight and concentrated to dryness. The residue was washed with a saturated NaHCO₃ aqueous solution and extracted thrice with EtOAc. The phases were separated and the organic phase was dried over Na₂SO₄ and concentrated to dryness. The crude product obtained was purified by HPLC to afford 11 mg of the title compound of the example (21% yield).

LC-MS (method 1): t_{R}=3.93 min; m/z=384 (MH⁺).

### Example 7

### 2-(4-Aminocarbonylphenyl)amino-6-[6-(4-aminopiperidin-1-yl)pyridin-3-yl]-9H-purine

### a) 6-[6-[4-(tert-Butoxycarbonyl)aminopiperidin-1-yl]pyridin-3-yl]-2-(4-ethoxycarbonylphenyl)amino-9-(tetrahydropyran-2-yl)-9H-purine

Following a similar procedure to that described in example 1 section b, but using ethyl 4-aminobenzoate instead of [4-(4-morpholino)phenyl]amine, the desired compound was obtained (34% yield).

LC-MS (method 1): t_{R}=10.54 min; m/z=643 (MH⁺).

### b) 6-[6-[4-(tert-Butoxycarbonyl)aminopiperidin-1-yl]pyridin-3-yl]-2-(4-carboxyphenyl)amino-9-(tetrahydropyran-2-yl)-9H-purine

To a solution of the compound obtained in the previous section (93 mg, 0.14 mmol) in EtOH (2 mL), a solution of KOH (54 mg, 0.96 mmol) in H₂O (2 mL) was added. The reaction mixture was stirred at 90° C. for 72 h. It was cooled until room temperature. The residue was washed with H₂O and extracted with EtOAc. The aqueous layer was cooled to 0° C. and adjusted to pH=4 by the addition of 1N HCl and it was extracted thrice with EtOAc. The combined organic phases were dried over anhydrous MgSO₄ and concentrated to dryness, to afford 60 mg (67% yield) of the desired compound.

LC-MS (method 2): t_{R}=2.27 min; m/z=615 (MH⁺).

### c) 2-(4-Aminocarbonylphenyl)amino-6-[6-[4-(tert-butoxycarbonyl)aminopiperidin-1-yl]pyridin-3-yl]-9-(tetrahydropyran-2-yl)-9H-purine

To a solution of the compound obtained in the previous section (60 mg, 0.09 mmol) in DMF (1.5 mL) under Ar-atmosphere, EDC.HCl (23 mg, 0.10 mmol), HOBT (13 mg, 0.09 mmol), NMM (43 μL, 0.36 mmol) and finally a 30% aqueous NH₃ solution (43 μL, 0.97 mmol) were added. The resulting mixture was stirred at room temperature overnight and concentrated to dryness. The resulting residue was diluted in a mixture EtOAc/H₂O (1:1), the phases were separated and the aqueous layer was extracted with EtOAc. The combined organic phases were dried over Na₂SO₄ and concentrated to dryness. The crude product obtained was chromatographed over silica gel using hexane/EtOAc mixtures of increasing polarity as eluent, to afford 46 mg of the desired compound (76% yield).

LC-MS (method 2): t_{R}=2.69 min; m/z=614 (MH⁺). 699/64

### d) Title Compound

Following a similar procedure to that described in example 1 section c, but using the compound obtained in the previous section, the title compound of the example was obtained (53% yield).

LC-MS (method 1): t_{R}=4.22 min; m/z=430 (MH⁺).

### Example 8

### 6-[3-(N-Isobutyl-N-acetylamino)phenyl]-2-[4-(4-morpholino)phenyl]amino-9H-purine

### a) N-Isobutyl-3-(4,4,5,5-tetramethyl-1,2,3-dioxaborolan-2-yl)aniline

To a solution of 3-(4,4,5,5-tetramethyl-1,2,3-dioxaborolan-2-yl)aniline (250 mg, 1.14 mmol) in CH₂Cl₂ (1 mL) under Ar-atmosphere, a solution of isobutyraldehyde (103 μL, 1.14 mmol) in CH₂Cl₂ (2 mL) was added. The resulting mixture was cooled to 0° C. and sodium triacetoxyborohydride (483 mg, 2.28 mmol) was added. The resulting mixture was stirred at room temperature overnight and diluted with EtOAc. It was treated with 0.2M NaHCO₃. The phases were separated and the aqueous phase extracted thrice with EtOAc. The combined organic phases were dried over Na₂SO₄ and concentrated to dryness. The crude product obtained was chromatographed over silica gel using hexane/EtOAc mixtures of increasing polarity as eluent, to afford 280 mg of the title compound (89% yield).

LC-MS (method 1): t_{R}=6.32 min; m/z=276 (MH⁺).

### b) 2-Chloro-6-[3-(N-isobutylamino)phenyl]-9-(tetrahydropyran-2-yl)-9H-purine

Following a similar procedure to that described in reference example 2, but using the compound obtained in the previous section instead of 2-fluoro-5-pyridylboronic acid, the desired compound was obtained (63% yield).

LC-MS (method 1): t_{R}=10.55 min; m/z=386 (MH⁺).

### c) 2-Chloro-6-[3-(N-isobutyl-N-acetylamino)phenyl]-9-(tetrahydropyran-2-yl)-9H-purine

To a solution of the compound obtained in the previous section (57 mg, 0.14 mmol) in CH₂Cl₂ (2 mL) under Ar-atmosphere, acetyl chloride (16 μL, 0.22 mmol) and DIEA (77 μL, 0.45 mmol) were added. The resulting mixture was stirred at room temperature overnight and concentrated to dryness. The resulting residue was diluted in a mixture of EtOAc/H₂O (1:1), the phases were separated and the aqueous phase extracted with EtOAc. The combined organic phases were dried over Na₂SO₄ and concentrated to dryness. The crude product obtained was chromatographed over silica gel using EtOAc/MeOH mixtures of increasing polarity as eluent, to afford 30 mg of the desired compound (47% yield).

LC-MS (method 1): t_{R}=9.52 min; m/z=428 (MH⁺).

### d) 6-[3-(N-Isobutyl-N-acetylamino)phenyl]-2-[4-(4-morpholino)phenyl]amino-9-(tetrahydropyran-2-yl)-9H-purine

Following a similar procedure to that described in example 1 section b, but starting from the compound obtained in the previous section, the desired compound was obtained (25% yield).

LC-MS (method 1): t_{R}=9.18 min; m/z=570 (MH⁺).

### e) Title Compound

Following a similar procedure to that described in example 1 section c, but using the compound obtained in the previous section, the title compound of the example was obtained (11% yield).

LC-MS (method 1): t_{R}=7.03 min; m/z=486 (MH⁺).

### Example 9

### 2-(3-Aminophenyl)amino-6-[6-(4-aminopiperidin-1-yl)pyridin-3-yl]-9H-purine

### a) 6-{6-[4-(tert-Butoxycarbonyl)aminopiperidin-1-yl]pyridin-3-yl}-2-(3-nitrophenyl)amino-9-(tetrahydropyran-2-yl)-9H-purine

Following a similar procedure to that described in example 1 section b, but using 3-nitroaniline instead of [4-(4-morpholino)phenyl]amine, the desired compound was obtained (56% yield).

LC-MS (method 2): t_{R}=3.45 min; m/z=616 (MH⁺).

### b) 2-(3-Aminophenyl)amino-6-{6-[4-(tert-butoxycarbonyl)aminopiperidin-1-yl]pyridin-3-yl}-9-(tetrahydropyran-2-yl)-9H-purine

Following a similar procedure to that described in reference example 7 section b, but starting from the compound obtained in the previous section, the desired compound was obtained (59% yield).

LC-MS (method 1): t_{R}=8.82 min; m/z=586 (MH⁺).

### c) Title Compound

Following a similar procedure to that described in example 1 section c, but using the compound obtained in the previous section, the title compound of the example was obtained (31% yield).

LC-MS (method 1): t_{R}=4.54 min; m/z=402 (MH⁺).

### Example 10

### 2-[4-(4-Morpholino)phenyl]amino-6-[6-(piperidin-3-ylamino)pyridin-3-yl]-9H-purine

### a) 2-[4-(4-morpholino)phenyl]amino-6-[6-[1-(tert-butoxycarbonyl)piperidin-3-ylamino]pyridin-3-yl]-9-(tetrahydropyran-2-yl)-9H-purine

Following a similar procedure to that described in example 2 section a, but using the compound obtained in example 5a section a, and 3-amino-1-(tert-butoxycarbonyl)piperidine, 0.027 g of the desired compound was obtained (20% yield).

### b) Title Compound

Following a similar procedure to that described in example 1 section c, but using the compound obtained in the previous section. In this case, the crude product obtained was chromatographed over a SCX-2 column instead of silica gel using MeOH—NH₃(MeOH) mixtures of increasing polarity as eluent. The title compound of the example was obtained (88% yield).

LC-MS (method 4): t_{R}=1.28 min; m/z=472 (MH⁺).

Following a similar procedure to that described in example 10, but using in each case the corresponding starting materials, these compounds were obtained:

| | | reagent for | HPLC | t_{R} | |
|---|---|---|---|---|---|
| Example | Compound name | step a) | method | (min) | m/z |
| 10a | 2-[4-(4-morpholino)phenyl]amino-6-[6- | N- | 4 | 1.55 | 431 |
| | (N-propylamino)pyridin-3-yl]-9H- | propylamine | | | |
| | purine | | | | |
| 10b | 6-[6-(N-dimethylamino)pyridin-3-yl]-2- | N- | 4 | 1.40 | 417 |
| | [4-(4-morpholino)phenyl]amino-9H- | dimethylamine | | | |
| | purine | hydrochloride | | | |
| 10c | 6-[6-([1,4]diazepan-1-yl)pyridin-3-yl]- | N-tert- | 4 | 1.28 | 472 |
| | 2-[4-(4-morpholino)phenyl]amino-9H- | butoxycarbonylhomopiperazine | | | |
| | purine | | | | |
| 10d | 6-[6-(3-methoxycarbonylpyrrolidin-1- | Methyl | 4 | 1.61 | 501 |
| | yl)pyridin-3-yl]-2-[4-(4- | pyrrolidine-3- | | | |
| | morpholino)phenyl]amino-9H-purine | carboxylate | | | |
| 10e | 6-[6-(N-ethylmethylamino)pyridin-3- | N- | 4 | 1.52 | 431 |
| | yl]-2-[4-(4-morpholino)phenyl]amino- | ethylmethylamine | | | |
| | 9H-purine | | | | |
| 10f | 6-[6-(N-butylmethylamino)pyridin-3- | N- | 4 | 1.88 | 459 |
| | yl]-2-[4-(4-morpholino)phenyl]amino- | butylmethylamine | | | |
| | 9H-purine | | | | |
| 10g | 6-[6-N-((2- | 2- | 4 | 1.32 | 447 |
| | hydroxyethyl)methylamino)pyridin-3- | (methylamino)ethanol | | | |
| | yl]-2-[4-(4-morpholino)phenyl]amino- | | | | |
| | 9H-purine | | | | |
| 10h | 6-[6-(N-diethylamino)pyridin-3-yl]-2-[4- | N- | 4 | 1.64 | 445 |
| | (4-morpholino)phenyl]amino-9H- | diethylamine | | | |
| | purine | | | | |
| 10i | 6-[6-(N-benzylmethylamino)pyridin-3- | N- | 4 | 2.19 | 493 |
| | yl]-2-[4-(4-morpholino)phenyl]amino- | benzylmethyl | | | |
| | 9H-purine | amine | | | |
| 10j | 6-[6-[(2-hydroxy-2- | 2- | 4 | 1.90 | 523 |
| | phenylethyl)methylamino]pyridin-3-yl]- | (methylamino)- | | | |
| | 2-[4-(4-morpholino)phenyl]amino-9H- | 1- | | | |
| | purine | phenylethanol | | | |
| 10k | 6-[6-(N-isobutylmethylamino)pyridin-3- | N- | 4 | 1.88 | 459 |
| | yl]-2-[4-(4-morpholino)phenyl]amino- | isobutymethyll | | | |
| | 9H-purine | amine | | | |
| 10l | 6-[6-(N-butylethylamino)pyridin-3-yl]- | N- | 4 | 2.03 | 473 |
| | 2-[4-(4-morpholino)phenyl]amino-9H- | butylethylamine | | | |
| | purine | | | | |
| 10m | 6-[6-((2- | 2- | 4 | 1.61 | 475 |
| | hydroxyethyl)propylamino)pyridin-3- | (propylamino)ethanol | | | |
| | yl]-2-[4-(4-morpholino)phenyl]amino- | | | | |
| | 9H-purine | | | | |
| 10n | 6-[6-(2- | N-(2- | 4 | 1.55 | 461 |
| | methoxyethyl)methylaminopyridin-3- | methoxyethyl)methylamine | | | |
| | yl]-2-[4-(4-morpholino)phenyl]amino- | | | | |
| | 9H-purine | | | | |
| 10o | 6-[6-(2- | 1- | 4 | 1.43 | 461 |
| | hydroxypropyl)methylaminopyridin-3- | (methylamino)propan- | | | |
| | yl]-2-[4-(4-morpholino)phenyl]amino- | 2-ol | | | |
| | 9H-purine | | | | |
| 10p | 6-[6-(N-ethylpropylamino)pyridin-3-yl]- | N- | 4 | 1.83 | 459 |
| | 2-[4-(4-morpholino)phenyl]amino-9H- | ethylpropylamine | | | |
| | purine | | | | |
| 10q | 6-[6-[N-methyl-(prop-2- | N- | 4 | 1.94 | 441 |
| | ynyl)amino]pyridin-3-yl]-2-[4-(4- | methylpropar | | | |
| | morpholino)phenyl]amino-9H-purine | gylamine | | | |
| 10r | 6-[6-(N-methylamino)pyridin-3-yl]-2-[4- | N- | 4 | 1.38 | 403 |
| | (4-morpholino)phenyl]amino-9H- | methylamine | | | |
| | purine | hydrochloride | | | |

### Example 11

### 6-[6-(3-Methylaminocarbonylpyrrolidin-1-yl)pyridin-3-yl]-2-[4-(4-morpholino)phenyl]amino-9H-purine

### a) 6-[6-(3-Carboxypyrrolidin-1-yl)pyridin-3-yl]-2-[4-(4-morpholino)phenyl]amino-9H-purine

Following a similar procedure to that described in example 7 section b, but using the compound obtained in example 10d, the title compound was obtained quantitatively.

### b) Title Compound

Following a similar procedure to that described in example 7 section c, but using the compound obtained in the previous section, and N-methylamine hydrochloride instead of 30% aqueous NH₃ solution, the title compound of the example was obtained (20% yield).

LC-MS (method 4): t_{R}=1.37 min; m/z=500 (MH⁺).

### Example 12

### 2-(3-Aminosulfonylphenyl)amino-6-{(4-[3-(hydroxymethyl)piperidin-1-yl]phenyl}-9H-purine

### a) 2-Chloro-6-{4-[3-(hydroxymethyl)piperidin-1-yl]phenyl}-9-(tetrahydropyran-2-yl)-9H-purine

Following a similar procedure to that described in reference example 2, but using the compound obtained in reference example 4a instead of 2-fluoro-5-pyridylboronic acid, the desired compound was obtained (39% yield).

LC-MS (method 5): t_{R}=2.47 min; m/z=428 (MH⁺).

### b) 2-[3-(N-tert-Butyl)aminosulfonylphenyl]amino-6-{4-[3-(hydroxymethyl)piperidin-1-yl]phenyl}-9-(tetrahydropyran-2-yl)-9H-purine

To a solution of the compound obtained in the previous section (150 mg, 0.351 mmol) in tert-butanol (4 mL) under Ar-atmosphere, potassium carbonate (106 mg, 0.768 mmol), X-Phos (17 mg, 0.036 mmol), Pd₂(dba)₃ (16 mg, 0.017 mmol) and 3-amino-N-tert-butylbenzenesulfonamide (160 mg, 0.701 mmol) were added at room temperature. The mixture was purgued under Ar-atmosphere and heated at 100° C. overnight. The reaction crude was filtered through a plug of Celite®, washed with methanol and evaporated to dryness. The crude product thus obtained was chromatographed over silica gel using hexane/EtOAc mixtures of increasing polarity as eluent, to afford 99 mg of the desired compound (46% yield).

LC-MS (method 5): t_{R}=2.53 min; m/z=620 (MH⁺)

### c) Title Compound

The compound obtained in the previous section (60 mg, 0.097 mmol) and a mixture of THF/6N HCl_{(aq)} (3 mL) was stirred at reflux temperature for 4 h under Ar-atmosphere. Afterwards, the mixture was concentrated to dryness and the residue was partitioned and the mixture was concentrated to dryness. The residue was partitioned between 0.2N NaHCO₃ and CH₂Cl₂. The organic phase was dried over Na₂SO₄ and concentrated to dryness. The crude product thus obtained was chromatographed over silica gel using CHCl₃/MeOH/NH₃ mixtures of increasing polarity as eluent, to afford 22 mg of the desired compound (48% yield).

LC-MS (method 1): t_{R}=5.83 min; m/z=480 (MH⁺).

Following a similar procedure to that described in example 12, but using the corresponding starting materials, the following compound was obtained:

| | | | | HPLC | t_{R} | |
|---|---|---|---|---|---|---|
| Example | Compound name | a) step reagent | b) step reagent | method | (min) | m/z |
| 12a | 2-(3- | 3- | 3-amino-N-tert- | 5 | 1.84 | 413 |
| | aminosufonylphenyl)amino- | (methylsulfanyl)phenylboronic | butylbenzenesulfonamide | | | |
| | 6-(3- | acid | | | | |
| | methylsulfanyl)phenyl- | | | | | |
| | 9H-purine | | | | | |

### Example 13

### 2-(3-Aminosulfonylphenyl)amino-6-[3-(methylsulfinyl)phenyl]-9H-purine

To a solution of the compound of example 12a (50 mg, 0.121 mmol) in 4 mL of a 1:1 mixture of acetic acid and methanol, was added under an Ar-atmosphere 0.04 mL of 30% H₂O₂ and the resulting mixture was stirred at room temperature overnight. The crude product obtained was evaporated to dryness and chromatographed over silica gel using CHCl₃/MeOH/NH₃ mixtures of increasing polarity as eluent, to afford 21 mg of the desired compound (41% yield).

LC-MS (method 5): t_{R}=1.27 min; m/z=429 (MH⁺).

Following a similar procedure to that described in example 13, but using the corresponding starting materials, the following compound was obtained:

| | | Starting | HPLC | t_{R} | |
|---|---|---|---|---|---|
| Example | Compound name | Material | method | (min) | m/z |
| 13a | 2-(3-acetylaminophenyl)- | Example | 5 | 1.37 | 407 |
| | amino-6-[3- | 1ch | | | |
| | (methylsulfinyl)phenyl]- | | | | |
| | 9H-purine | | | | |

### Example 14

### 2-(3-Aminosulfonylphenyl)amino-6-[4-(ethylaminocarbonyloxy)phenyl]-9H-purine

### a) 2-Chloro-6-(4-hydroxyphenyl)-9-(tetrahydropyran-2-yl)-9H-purine

Following a similar procedure to that described in reference example 2, but using 4-hydroxyphenyl boronic acid instead of 2-fluoro-5-pyridylboronic acid, the desired compound was obtained (21% yield).

LC-MS (method 5): t_{R}=2.11 min; m/z=329 (MH⁻).

### b) 2-Chloro-6-(4-ethylaminocarbonyloxy)phenyl-9-(tetrahydropyran-2-yl)-9H-purine

A mixture of the compound obtained in the previous section (125 mg, 0.378 mmol), ethyl isocyanate (0.030 mL, 0.380 mmol) and 3 mL of DMF was stirred at 80° C. overnight. The resulting solution was evaporated to dryness and chromatographed over silica gel using hexane/EtOAc mixtures of increasing polarity as eluent, to afford 33 mg of the desired compound (22% yield).

LC-MS (method 5): t_{R}=2.36 min; m/z=402 (MH⁺)

### c) 2-(3-Aminosulfonylphenyl)amino-6-(4-ethylaminocarbonyloxy)phenyl-9-(tetrahydropyran-2-yl)-9H-purine

Following a similar procedure to that described in example 12 section b, but using the compound obtained in the previous section and 3-aminobenzenesulfonamide instead of 3-amino-N-tert-butylbenzenesulfonamide, the desired compound was obtained.

LC-MS (method 5): t_{R}=1.40 min; m/z=538 (MH⁺).

### d) Title Compound

A mixture of the compound obtained in the previous section (68 mg, 0.126 mmol), 4M dioxane/HCl_{(g)} (5 mL) and 1 mL of methanol was stirred at room temperature under Ar-atmosphere overnight. The solution was concentrated to dryness and the crude product thus obtained was chromatographed over silica gel using EtOAc/MeOH mixtures of increasing polarity as eluent, to afford 27 mg of the desired compound (47% yield).

LC-MS (method 1): t_{R}=3.83 min; m/z=454 (MH⁺).

Following a similar procedure to that described in example 14, but using the corresponding starting materials, the following compound was obtained:

| | | reagent for | reagent for | HPLC | t_{R} | |
|---|---|---|---|---|---|---|
| Example | Compound name | step a) | step c) | method | (min) | m/z |
| 14a | 2-[3- | 4- | 3- | 5 | 1.43 | 453 |
| | aminosulfonylphenyl]amino- | aminophenylboronic | aminobenzenesulfonamide | | | |
| | 6-[4- | acid | | | | |
| | (ethylaminocarbonylamino)phenyl]- | | | | | |
| | 9H-purine | | | | | |

### Example 15

### 2-[3-(Aminosulfonyl)phenyl]amino-6-[4-(methanesulfonylamino)phenyl]-9H-purine

### a) 6-(4-Aminophenyl)-2-chloro-9-(tetrahydropyran-2-yl)-9H-purine

Following a similar procedure to that described in reference example 2, but using 4-(4,4,5,5-tetramethyl-1,3,2-dioxaborolan-2-yl)aniline instead of 2-fluoro-5-pyridylboronic acid, the desired compound was obtained (33% yield).

LC-MS (method 5): t_{R}=2.15 min; m/z=330 (MH⁺).

### b) 2-Chloro-6-[4-(methanesulfonylamino)phenyl]-9-(tetrahydropyran-2-yl)-9H-purine

To a mixture of the compound obtained in the previous section (170 mg, 0.52 mmol), a catalytic amount of DMAP, diisopropyethylamine (0.181 mL, 1.04 mmol) and 4 mL dichloromethane, methanesulfonyl chloride (40.3 4, 0.52 mmol) was added and the resulting mixture was stirred at room temperature overnight. The resulting solution was partitioned between H₂O and dichloromethane and the organic phase was dried over Na₂SO₄ and concentrated to dryness. The crude product thus obtained was chromatographed over silica gel using EtOAc/MeOH mixtures of increasing polarity as eluent, to afford 14 mg of the desired compound (7% yield).

LC-MS (method 5): t_{R}=2.07 min; m/z=408 (MH⁺)

### c) 2-(3-Aminosulfonylphenyl)amino-6-[4-(methanesulfonylamino)phenyl]-9-(tetrahydropyran-2-yl)-9H-purine

Following a similar procedure to that described in example 12 section b, but using the compound obtained in the previous section and 3-aminobenzenesulfonamide instead of 3-amino-N-tert-butylbenzenesulfonamide, the desired compound was obtained.

LC-MS (method 5): t_{R}=1.78 min; m/z=544 (MH⁺).

### d) Title Compound

Following a similar procedure to that described in example 14 section d, but using the compound obtained in the previous section, the title compound of the example was obtained (26% yield).

LC-MS (method 5): t_{R}=1.28 min; m/z=460 (MH⁺).

Following a similar procedure to that described in example 15, but using in each case the corresponding starting materials, these compounds were obtained:

| | Compound | reagent | reagent | reagent for | HPLC | t_{R} | |
|---|---|---|---|---|---|---|---|
| Example | name | for step a) | for step b) | step c) | method | (min) | m/z |
| 15a | 2-(3- | 4-(4,4,5,5- | isobutyril | 3-amino-N- | 5 | 1.58 | 452 |
| | aminosulfonylphenyl)amino- | tetramethyl- | chloride | tert- | | | |
| | 6-[4-(N- | 1,3,2- | | butylbenzene | | | |
| | isobutirylamino)phenyl]- | dioxaborolan- | | sulfonamide | | | |
| | 9H-purine | 2- | | | | | |
| | | yl)aniline | | | | | |
| 15b | 6-[4-(N- | N-methyl- | propionyl | [4-(4- | 4 | 2.13 | 458 |
| | methylpropionylamino)phenyl]- | 4-(4,4,5,5- | chloride | morpholino)phenyl]amine | | | |
| | 2-[4-(4- | tetramethyl- | | | | | |
| | morpholino)phenyl]amino- | 1,3,2- | | | | | |
| | 9H-purine | dioxaborolan- | | | | | |
| | | 2- | | | | | |
| | | yl)aniline | | | | | |
| 15c | 6-[4-(N- | N-methyl- | methanesulfonyl | [4-(4- | 4 | 2.15 | 480 |
| | methylmethanesulfonylamino)- | 4-(4,4,5,5- | chloride | morpholino)phenyl]amine | | | |
| | phenyl]-2- | tetramethyl- | | | | | |
| | [4-(4- | 1,3,2- | | | | | |
| | morpholino)phenyl]amino- | dioxaborolan- | | | | | |
| | 9H-purine | 2- | | | | | |
| | | yl)aniline | | | | | |
| 15d | 2-[4-(4- | N-tert- | propionyl | [4-(4- | 4 | 1.64 | 471 |
| | methylpiperazin- | butoxycarbonyl- | chloride | methylpiperazin- | | | |
| | 1- | N- | | 1- | | | |
| | yl)phenyl]amino- | methyl-4- | | yl)phenyl]amine | | | |
| | 6-[4-(N- | (4,4,5,5- | | | | | |
| | methylpropionylamino)phenyl]- | tetramethyl- | | | | | |
| | 9H- | 1,3,2- | | | | | |
| | purine | dioxaborolan- | | | | | |
| | | 2- | | | | | |
| | | yl)aniline (1) | | | | | |
| 15e | 6-[4-(N- | N-tert- | methanesulfonyl | [4-(4- | 4 | 1.63 | 493 |
| | methylmethanesulfonylamino)- | butoxycarbonyl- | chloride | methylpiperazin- | | | |
| | phenyl]-2- | N- | | 1- | | | |
| | [4-(4- | methyl-4- | | yl)phenyl]amine | | | |
| | methylpiperazin- | (4,4,5,5- | | | | | |
| | 1- | tetramethyl- | | | | | |
| | yl)phenyl]amino- | 1,3,2- | | | | | |
| | 9H-purine | dioxaborolan- | | | | | |
| | | 2- | | | | | |
| | | yl)aniline (1) | | | | | |

(1) Step d was performed after step a

### Example 16

### 6-[6-(N-Methylmethanesulfonylamino)pyridin-3-yl]-2-[4-(4-morpholino)phenyl]amino-9H-purine

### a) 6-[6-(N-Methylmethansulfonylamino)pyridin-3-yl]-2-[4-(4-morpholino)phenyl]amino-9-(tetrahydropyran-2-yl)-9H-purine

Following a similar procedure to that described in example 15 section b, but using the compound obtained in example 10r section b, the desired compound was obtained.

### b) Title Compound

Following a similar procedure to that described in example 14 section d, but using the compound obtained in the previous section, the title compound of the example was obtained.

LC-MS (method 4): t_{R}=2.15 min; m/z=481 (MH⁺).

### Example 17

### 2-[3-(N-Acetyl)aminosulfonylphenyl]amino-6-[6-(methylpropylamino)pyridin-3-yl]-9H-purine

A mixture of the compound of example 2r (115 mg, 0.26 mmol), N,N-dimethyaminopyridine (catalytic amount), acetic anhydride (0.025 mL, 0.26 mmol) and pyridine (4 mL) was stirred at room temperature overnight. The resulting solution was evaporated to dryness and chromatographed over silica gel using CHCl₃/MeOH/NH₃ mixtures of increasing polarity as eluent, to afford 17 mg of the desired compound (13% yield).

LC-MS (method 5): t_{R}=1.49 min; m/z=481 (MH⁺).

### Example 18

### 2-(3-Aminosulfonylphenyl)amino-6-[6-(3-hydroxypiperidin-1-yl)pyridin-3-yl]-9H-purine

### a) 6-[6-[3-(tert-Butyldimethylsilyloxy)piperidin-1-yl]pyridin-3-yl]-2-chloro-9-(tetrahydropyran-2-yl)-9H-purine

A mixture of the compound obtained in example 2a section a (1.84 g, 4.42 mmol), imidazole (752 mg, 11.05 mmol), tert-butyldimethylsilyl chloride and DMF (50 mL) was stirred at room temperature overnight. The resulting solution was diluted with dichloromethane (250 mL) and was partitioned between H₂O and dichloromethane. The organic phase was dried over Na₂SO₄ and concentrated to dryness. The crude product thus obtained was chromatographed over silica gel using EtOAc/MeOHI mixtures of increasing polarity as eluent, to afford the desired compound in quantitative yield.

### b) 6-[6-[3-(tert-Butyldimethylsilyloxy)piperidin-1-yl]pyridin-3-yl]-2-(3-tert-butylaminosulfonylphenyl)amino-9-(tetrahydropyran-2-yl)-9H-purine

Following a similar procedure to that described in example 12 section b, but using the compound obtained in the previous section instead of 2-chloro-6-{4-[3-(hydroxymethyl)piperidin-1-yl]phenyl}-9-(tetrahydropyran-2-yl)-9H-purine, the title compound was obtained.

### c) 2-(3-tert-Butylaminosulfonylphenyl)amino-6-[6-(3-hydroxypiperidin-1-yl)pyridin-3-yl]-9-(tetrahydropyran-2-yl)-9H-purine

To a solution of the compound obtained in the previous section (136 mg, 0.19 mmol) and 3.8 mL of THF, tetrabutylammonium fluoride hydrate (148 mg, 056 mmol) was added. The mixture was stirred for 3 h at room temperature and the resulting suspension was evaporated to dryness and chromatographed over silica gel using CHCl₃/MeOH/NH₃ mixtures of increasing polarity as eluent, to afford 82 mg of the desired compound (72% yield).

### d) Title Compound

Following a similar procedure to that described in example 12 section c, but using the compound obtained in the previous section, the title compound was obtained.

LC-MS (method 4): t_{R}=1.45 min; m/z=467 (MH⁺).

Following a similar procedure to that described in example 18, but using in each case the corresponding starting materials, these compounds were obtained:

| | | | HPLC | | |
|---|---|---|---|---|---|
| Example | Compound name | reagent for step b) | method | t_{R} (min) | m/z |
| 18a | 6-[6-(3-hydroxypiperidin- | 3-amino-N- | 4 | 1.61 | 481 |
| | 1-yl)pyridin-3-yl]-2-(3- | methylbenzenesulfonamide | | | |
| | methylaminosulfonylbenzene)amino- | | | | |
| | 9H-purine | | | | |
| 18b | 2-[4-(3-aminopyrrolidin- | Reference Example | 4 | 1.19 | 472 |
| | 1-yl)phenyl]amino-6-[6- | 8a | | | |
| | (3-hydroxypiperidin-1- | | | | |
| | yl)pyridin-3-yl]-9H-purine | | | | |

### Example 19

### 6-(6-Butoxypyridin-3-yl)-2-[4-(4-morpholino)phenyl]amino-9H-purine

### a) 6-(6-Butoxypyridin-3-yl)-2-[4-(4-morpholino)phenyl]amino 9-(tetrahydropyran-2-yl)-9H-purine

A mixture of the compound obtained in example 5a section a (100 mg, 0.21 mmol) and potassium tert-butoxyde (56 mg, 0.5 mmol) in n-BuOH (2 mL) was irradiated in a monomode microwave at 160° C. for 10 min (160 W). The resulting crude product was evaporated to dryness and was partitioned between H₂O and dichloromethane. The organic phase was dried over Na₂SO₄ and concentrated to dryness and the crude product thus obtained was chromatographed over silica gel using CHCl₃/MeOH/NH₃ mixtures of increasing polarity as eluent, to afford 54 mg of the desired compound.

### b) Title Compound

Following a similar procedure to that described in example 1 section c, but using the compound obtained in the previous section, the title compound of the example was obtained.

LC-MS (method 4): t_{R}=2.97 min; m/z=446 (MH⁺).

Following a similar procedure to that described in example 19, but using the corresponding starting materials, the following compound was obtained:

| | | reagent for | HPLC | t_{R} | |
|---|---|---|---|---|---|
| Example | Compound name | step b) | method | (min) | m/z |
| 19a | 6-[6-(2-hydroxy)- | ethylene glycol | 4 | 1.70 | 434 |
| | ethoxypyridin- | | | | |
| | 3-yl]-2-[4-(4- | | | | |
| | morpholino)- | | | | |
| | phenyl]amino-9H- | | | | |
| | purine | | | | |

### Example 20

### 2-(3-Aminosulfonylphenyl)amino-6-(2-carboxypyrrole-4-yl)-9H-purine

### a) 2-Chloro-9-(tetrahydropyran-2-yl)-6-[2-(methoxycarbonyl)-1-(4-toluoyl)sulfonyl-pyrrole-4-yl]-9H-purine

Following a similar procedure to that described in reference example 2, but using the compound obtained in reference example 11 instead of 2-fluoro-5-pyridylboronic acid, and using K₂CO₃ instead of Na₂CO₃, the desired compound was obtained (60% yield).

LC-MS (method 5): t_{R}=3.02 min; m/z=516 (MH⁺).

### b) 2-(3-Aminosulfonyl)phenylamino-9-(tetrahydropyran-2-yl)-6-[1-(4-toluoyl)sulfonyl-2-(methoxycarbonyl)pyrrole-4-yl]-9H-purine

Following a similar procedure to that described in example 12 section b, but using the compound obtained in the previous section instead of 2-chloro-6-{4-[3-(hydroxymethyl)piperidin-1-yl]phenyl}-9-(tetrahydropyran-2-yl)-9H-purine and 3-aminobenzenesulfonamide instead of 3-amino-N-tert-butylbenzenesulfonamide, the desired compound was obtained (72% yield).

LC-MS (method 5): t_{R}=2.60 min; m/z=652 (MH⁺).

### c) Title Compound

A solution of the compound obtained in the previous section (0.54 g, 0.83 mmol) in 30 mL methanol and 25 mL 1N NaOH was heated at 80° C. during 2 h. A solution of 6N HCl was added dropwise until acidic pH and the solution extracted three times with ethyl acetate. The organic layer was dried over Na₂SO₄ and evaporated to dryness. The crude product thus obtained was filtered over silica gel using CHCl₃/MeOH/acetic acid/DMF mixtures as eluent, the solution was evaporated and dried, to afford the title compound (17% yield).

LC-MS (method 5): t_{R}=0.82 min; m/z=400 (MH⁺).

### Example 21

### 2-(3-Aminophenyl)amino-6-(3-methanesulfonyl)phenyl-9H-purine

### a) 2-(3-Aminophenyl)amino-6-(3-methanesulfonyl)phenyl-9-(tetrahydropyran-2-yl)-9H-purine

Following a similar procedure to that described in reference example 7 section b, but using the compound obtained in 1cu section b, the desired compound was obtained in quantitative yield.

### b) Title Compound

Following a similar procedure to that described in example 1 section c, but using the compound obtained in the previous section, the title compound of the example was obtained (5% yield).

LC-MS (method 4): t_{R}=1.82 min; m/z=381 (MH⁺).

### Example 22

### 6-(3-Methylsulfanylphenyl)-2-[4-(4-morpholino)phenyl]amino-9H-purine

### a) 2-Chloro-6-[(3-methylsulfanyl)phenyl]-9-(tetrahydropyran-2-yl)-9H-purine

Following a similar procedure to that described in reference example 2, but using 3-(methylsulfanyl)phenylboronic acid instead of 2-fluoro-5-pyridylboronic acid, the desired compound was obtained (72% yield).

LC-MS (method 5): t_{R}=2.18 min; m/z=419 (MH⁺).

### b) 2-Chloro-6-[(3-methylsulfinyl)phenyl]-9-(tetrahydropyran-2-yl)-9H-purine

To a solution of the compound obtained in the previous section in dichloromethane (2 mL) 197 mg of m-chloroperbenzoic acid (77%) was added. The mixture was stirred overnight at room temperature and then the solvent was evaporated. The crude product thus obtained was purified over silica gel using hexane/EtOAc mixtures of increasing polarity, to afford 195 mg of the title compound (70% yield).

LC-MS (method 5): t_{R}=1.95 min; m/z=377 (MH⁺).

### c) 6-[(3-Methylsulfinyl)phenyl]-2-[4-(4-morpholino)phenyl]amino-9-(tetrahydropyran-2-yl)-9H-purine

To a solution of the compound obtained in the previous section (97 mg, 0.258 mmol) in tert-butanol (5 mL), K₂CO₃ (157 mg, 0.567 mmol), X-Phos (25 mg, 0.0258 mmol), Pd₂(dba)₃ (24 mg, 0.0129 mmol) and [4-(4-morpholino)phenyl]amine (184 mg, 1.034 mmol) were added at room temperature and the mixture was stirred under Ar-atmosphere at 90° C. overnight. The crude product obtained was filtered over Celite® and concentrated to dryness.

LC-MS (method 5): t_{R}=1.99 min; m/z=519 (MH⁺).

### d) Title Compound

The crude product obtained in the previous section (0.258 mmol) and a mixture of 4M dioxane/HCl_{(g)} (3 mL) was stirred at room temperature under Ar-atmosphere overnight. The solvent was concentrated and the crude product obtained was chromatographed over silica gel using CHCl₃/MeOH/NH₃ mixtures of increasing polarity as eluent, to afford the desired compound (7% yield).

LC-MS (method 5): t_{R}=2.18 min; m/z=419 (MH⁺).

### Example 23

### 2-(3-Aminosulfonyl)phenylamino-6-(4-pyrazolyl)-9H-purine

### a) 2-Chloro-6-(1-tert-butoxycarbonylpyrazol-4-yl)-9-(tetrahydropyran-2-yl)-9H-purine

Following a similar procedure to that described in reference example 2, but using 2-(1-tert-butoxycarbonyl)pyrazol-4-yl-4,4,5,5-tetramethyl-[1,3,2]dioxaborolane instead of 2-fluoro-5-pyridylboronic acid, the desired compound was obtained.

LC-MS (method 5): t_{R}=1.70 min; m/z=303 (MH⁻).

### b) 2-[(3-Aminosulfonyl)phenyl]amino-6-(4-pyrazolyl)-9-(tetrahydropyran-2-yl)-9H-purine

Following a similar procedure to that described in example 22 section c, but using the compound obtained in the previous section, and 3-aminobenzenesulfonamide instead of [4-(4-morpholino)phenyl]amine, the desired compound was obtained.

LC-MS (method 1): t_{R}=5.63 min; m/z=441 (MH⁺).

### c) Title Compound

The compound obtained in the previous section was mixed with Dowex 50w×8 (440 mg) in methanol (4 mL) and DMSO (1 mL) and the mixture was stirred overnight at room temperature. The suspension was filtered and washed with NH₄OH/MeOH (25%) and methanol. Evaporation of the solvent yielded the desired product.

LC-MS (method 1): t_{R}=4.01 min; m/z=357 (MH⁺).

Following a similar procedure to that described in example 23, but using in each case the corresponding starting materials, the following compounds were obtained:

| | | reagent for | reagent for | HPLC | t_{R} | |
|---|---|---|---|---|---|---|
| Example | Compound name | step a) | step b) | method | (min) | m/z |
| 23a | 2-[(3- | 1-methyl-4- | 3- | 5 | 1.18 | 371 |
| | aminosulfonyl)phenyl]amino- | (4,4,5,5- | aminobenzenesulfonamide | | | |
| | 6-(1-methyl-4- | tetramethyl- | | | | |
| | pyrazolyl)-9H-purine | 1,3,2- | | | | |
| | | dioxaborolan-2- | | | | |
| | | yl)-1H-pyrazole | | | | |
| 23b | 2-[(3- | 3-furanboronic | 3- | 5 | 5.38 | 357 |
| | aminosulfonyl)phenyl]amino- | acid | aminobenzenesulfonamide | | | |
| | 6-(3-furanyl)-9H- | | | | | |
| | purine | | | | | |
| 23c | 2-[(3- | 1-(triisopropyl- | 3- | 5 | 1.18 | 356 |
| | aminosulfonyl)phenyl]amino- | silyl)-1H- | aminobenzenesulfonamide | | | |
| | 6-(3-pyrrolyl)-9H- | pyrrole-3- | | | | |
| | purine | boronic acid | | | | |

### Example 24

### 6-[1-(Aminocarbonyldimethylmethyl)pyrazol-4-yl]-2-(3-aminosulfonyl)phenylamino-9H-purine

### a) 2-Chloro-6-[1-(ethoxycarbonyldimethylmethyl)pyrazol-4-yl]-9-(tetrahydropyran-2-yl)-9H-purine

To a solution of the compound obtained in example 23, section a in DMF (16 mL), cooled to 0° C., 171 mg (3.938 mmol) of NaH and ethyl-2-bromoisobutyrate (0.442 mL, 2.953 mmol) were added. The mixture was stirred for 3 h at room temperature. The resulting suspension was diluted with a mixture of tert-butylmethyl ether (100 mL), water (20 mL) and NH₄Cl saturated solution (5 mL). The two phases were separated and the aqueous phase was extracted with tert-butylmethyl ether. The combined organic phases were dried over Na₂SO₄ and concentrated to dryness to afford the desired product.

### b) 6-[1-(Carboxydimethylmethyl)pyrazol-4-yl]-2-chloro-9-(tetrahydropyran-2-yl)-9H-purine

To a solution of 378 mg of the compound obtained in the previous section in THF (2 mL), a solution of LiOH.H₂O (75 mg) in 2 mL of water was added. The mixture was stirred overnight at room temperature. The crude product was cooled to 0° C. and 2 mL HCl 1N, 2.5 mL of water and 50 mL of EtOAc were added. The phases were separated and the aqueous phase extracted with EtOAc. The combined organic phases were dried over Na₂SO₄ and concentrated to dryness, to afford. 347 mg of the desired product.

LC-MS (method 5): t_{R}=1.41 min; m/z=391 (MH⁺).

### c) 6-[1-(Aminocarbonyldimethylmethyl)pyrazol-4-yl]-2-chloro-9-(tetrahydropyran-2-yl)-9H-purine

A mixture of the product obtained in the previous section (144 mg, 0.346 mmol) and CU (100 mg, 0.554 mmol) in 8 mL of DMF was stirred for 3 h at room temperature. Then, triethylamine (0.217 mL, 1.55 mmol) and ammonium chloride (56 mg, 1.04 mmol) were added and the mixture was stirred overnight at room temperature. The resulting suspension was diluted in EtOAc and washed with 1N HCl, water, 1N NaOH and brine. The organic phase was dried over Na₂SO₄ and concentrated to dryness, to afford 112 mg of the desired product.

LC-MS (method 5): t_{R}=1.82 min; m/z=390 (MH⁺).

### d) 6-[1-(Aminocarbonyldimethylmethyl)pyrazol-4-yl]-2-(3-aminosulfonyl)phenylamino-9-(tetrahydropyran-2-yl)-9H-purine

Following a similar procedure to that described in 22 section c, but using the compound obtained in the previous section, and 3-aminobenzenesulfonamide instead of [4-(4-morpholino)phenyl]amine, the desired compound was obtained.

LC-MS (method 5): t_{R}=1.66 min; m/z=526 (MH⁺).

### e) Title Compound

A mixture of the compound obtained in the previous section and 4M dioxane/HCl_{(g)} (2 mL) was stirred at room temperature overnight. The suspension was concentrated to dryness and the crude product thus obtained was chromatographed over silica gel using CHCl₃/MeOH mixtures of increasing polarity as eluent, to afford the desired compound.

LC-MS (method 5): t_{R}=1.24 min; m/z=442 (MH⁺).

Following a similar procedure to that described in example 24, but using in each case the corresponding starting materials, the following compound was obtained:

| | | | HPLC | | |
|---|---|---|---|---|---|
| Example | Compound name | reagent for step d) | method | t_{R} (min) | m/z |
| 24a | 6-[1- | [4-(4- | 5 | 1.32 | 461 |
| | (aminocarbonyldimethylmethyl)pyrazol- | methylpiperazin-1- | | | |
| | 4-yl]-2-[4-(4- | yl)phenyl]amine | | | |
| | methylpiperazin-1- | | | | |
| | yl)phenyl]amino-9H-purine | | | | |

### Example 25

### 2-(3-Aminosulfonyl)phenylamino-6-[3-(2,2,2-trifluoroethyl)aminocarbonylphenyl]-9H-purine

### a) 6-(3-Carboxy)phenyl-2-chloro-6-(3-carboxy)phenyl-9-(tetrahydropyran-2-yl)-9H-purine

Following a similar procedure to that described in reference example 2, but using 3-carboxyphenylboronic acid instead of 2-fluoro-5-pyridylboronic acid, the desired compound was obtained (90% yield).

### b) 2-Chloro-9-(tetrahydropyran-2-yl)-6-[3-(2,2,2-trifluoroethyl)aminocarbonylphenyl]-9H-purine

A mixture of the compound obtained in the previous section (420 mg, 1.17 mmol), DIEA (0.92 mL, 5.26 mmol), 2,2,2-trifluoroethylamine hydrochloride (476 mg, 3.51 mmol) and HBTU (533 mg, 1.40 mmol) were stirred at room temperature in 30 mL DMF overnight. The mixture was evaporated to dryness and chromatographed over silica gel using Hexane/Ethyl Acetate mixtures of increasing polarity as eluent, to afford the desired compound (26%).

LC-MS (method 5): t_{R}=2.46 min; m/z=440 (MH⁺).

### c) 2-(3-Aminosulfonyl)phenylamino-9-(tetrahydropyran-2-yl)-6-[3-(2,2,2-trifluoroethyl)aminocarbonylphenyl]-9H-purine

Following a similar procedure to that described in 12 section b, but using the compound obtained in the previous section, and 3-aminobenzenesulfonamide instead of 3-amino-N-tert-butylbenzenesulfonamide, the desired compound was obtained.

LC-MS (method 5): t_{R}=2.14 min; m/z=576 (MH⁺).

### d) Title Compound

A mixture of the compound obtained in the previous section (0.305 mmol) and 4M dioxane/HCl_{(g)} (5.2 mL) was stirred at room temperature under Ar-atmosphere overnight. The solution was concentrated to dryness and the crude product thus obtained was chromatographed over silica gel using CHCl₃/MeOH/NH₃ mixtures of increasing polarity as eluent, to afford 77 mg of the desired compound (51% yield).

LC-MS (method 5): t_{R}=1.71 min; m/z=492 (MH⁺).

### Example 26

### 2-(3-Aminosulfonylphenyl)amino-6-(2-methylaminocarbonylpyrrole-4-yl)-9H-purine

A mixture of the compound obtained in example 20 (20 mg, 0.041 mmol), HBTU (19 mg, 0.050 mmol), and methylamine solution 2.0M in THF (0.1 mL, 0.207 mmol) were stirred in 1 mL of DMF at room temperature overnight. The resulting mixture was evaporated to dryness and purified over preparative HPLC. The title compound was obtained (3%).

LC-MS (method 5): t_{R}=1.71 min; m/z=492 (MH⁺).

Following a similar procedure to that described in example 26, but using the corresponding starting material, the following compound was obtained:

| | | | HPLC | | |
|---|---|---|---|---|---|
| Example | Compound name | reagent | method | t_{R} (min) | m/z |
| 26a | 2-(3- | Ethylamine (2.0M in | 5 | 1.35 | 427 |
| | aminosulfonylphenyl)amino-6- | THF) | | | |
| | (2-ethylaminocarbonylpyrrole-4- | | | | |
| | yl)-9H-purine | | | | |

### Example 27

### Biological Assay 1

### JAK3 Kinase Inhibition

In a final volume of 50 μL, 5 μL of the test product dissolved in 10% DMSO (final concentration, 0.001-10 μM), was incubated with 4 μg/mL of human JAK3 781-1124, 1 μg/mL of Poly-L-Ala, L-Glu, L-Lys, L-Tyr and ATP (0.2 μM, approximately 2×10⁵ cpm of γ³³P-ATP) in HEPES buffer (60 mM, pH 7.5) with Mg²⁺chloride (3 mM), Mn²⁺ chloride (3 mM), sodium orthovanadate (3 μM) and dithiotreitol (1.2 mM). The reaction was started by adding Mg²⁺[γ³³P-ATP]. After incubation for 50 min at room temperature, the reaction was quenched by the addition of 50 μL of 2% phosphoric acid solution. The reaction mixture was filtered in vacuo and washed three times with a 150 mM phosphoric acid solution. 200 μL of liquid scintillation was added before drying it and counting it.
The compounds of all examples showed more than 50% of inhibition of JAK3 activity at 10 μM in this assay.

### Example 28

### Biological Assay 2

### Delayed-Type Hypersensitivity Response (DTH)

This assay was performed essentially as disclosed in Kudlacz E. et al, see supra. Male C57BL/6J mice received i.v. injections of 1×10⁵ sheep red blood cells in a volume of 0.2 mL sterile phosphate buffered saline (PBS). Four days later, sensitized mice received an injection of 1×10⁸ sheep red blood cells in a volume of 30 μL sterile PBS into the left footpad. Twenty-four hours later, animals were sacrificed and their footpads removed and weighted. The DTH swelling response was calculated by subtracting the right footpad weight (baseline) from that of the left footpad (experimental). Test compounds or vehicle (0.2% carboxymethylcellulose and 1% Tween 80 in water) were administered p.o. once daily during both sensitization and challenge phases of the DTH response.
Compounds of examples 1 cc, 1cr, 1ct, 5u, 10h and 10p were active in this assay when administered orally.

1. A compound of formula I: 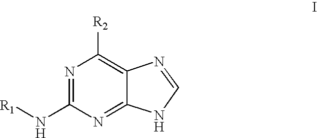
wherein:
R₁ is chosen from phenyl and a 5- or 6-membered aromatic heterocycle bonded to the NH group through a C atom, wherein the phenyl or heterocycle is optionally fused to a 5- or 6-membered saturated, partially unsaturated or aromatic carbocyclic or heterocyclic ring, wherein R₁ optionally contains from 1 to 4 heteroatoms chosen from N, O and S, wherein one or more C or S atoms of the 5- or 6-membered fused ring are optionally oxidized forming CO, SO or SO₂ groups, and wherein R₁ is optionally substituted with one or more R₃;
R₂ is chosen from phenyl and a 5- or 6-membered aromatic heterocycle bonded to the purine ring through a C atom, wherein the phenyl or heterocycle is optionally fused to a 5- or 6-membered saturated, partially unsaturated or aromatic carbocyclic or heterocyclic ring, wherein R₂ optionally contains from 1 to 4 heteroatoms chosen from N, O and S, wherein one or more C or S atoms of the 5- or 6-membered fused ring is optionally oxidized forming CO, SO or SO₂ groups, and wherein R₂ is optionally substituted with one or more R₄;
R₃ and R₄ independently are chosen from C₁₋₄alkyl, C₂₋₄alkenyl, C₂₋₄alkynyl, halogen, —CN, —NO₂, —COR_{E}, —CO₂R₆, —CONR₆R₆, —OR₆, —OCOR₅, —OCONR₅R₅, —OCO₂R₅, —SR₆, —SO₂R₅, —SO₂NR₆R₆, —SO₂NR₇COR₅, —NR₆R₆, —NR₇COR₆, —NR₇CONR₆R₆, —NR₇CO₂R₅, —NR₇SO₂R₅, —C(═N—OH)R₅, and Cy₁, wherein the C₁₋₄alkyl, C₂₋₄alkenyl and C₂₋₄alkynyl groups are optionally substituted with one or more R₈ and Cy₁ is optionally substituted with one or more R₉;
R₅ is chosen from C₁₋₄alkyl, C₂₋₄alkenyl, C₂₋₄alkynyl, and Cy₂, wherein the C₁₋₄alkyl, C₂₋₄alkenyl and C₂₋₄alkynyl groups are optionally substituted with one or more R₁₀ and Cy₂ is optionally substituted with one or more R₁₁;
R₆ is chosen from hydrogen and R₅;
R₇ is chosen from hydrogen and C₁₋₄alkyl;
R₈ is chosen from halogen, —CN, —NO₂, —COR₁₃, —CO₂R₁₃, —CONR₁₃R₁₃, —OR₁₃, —OCOR₁₂, —OCONR₁₂R₁₂, —OCO₂R₁₂, —SR₁₃, —SO₂R₁₂, —SOR₁₂, —SO₂NR₁₃R₁₃, —SO₂NR₇COR₁₂, —NR₁₃R₁₃, —NR₇COR₁₃, —NR₇CONR₁₃R₁₃, —NR₇CO₂R₁₂, —NR₇SO₂R₁₂, —C(═N—OH)R₁₂ and Cy₂, wherein Cy₂ is optionally substituted with one or more R₁₁;
R₉ is chosen from the group of R₁₄ and C₁₋₄alkyl, where the C₁₋₄alkyl is optionally substituted with one or more R₁₀;
R₁₀ is chosen from halogen, —CN, —NO₂, —COR₁₆, —CO₂R₁₆, —CONR₁₆R₁₆, —OR₁₆, —OCOR₁₅, —OCONR₁₅R₁₅, —OCO₂R₁₅, —SR₁₆, —SO₂R₁₅, —SOR₁₅, —SO₂NR₁₆R₁₆, —SO₂NR₇COR₁₅, —NR₁₆R₁₆, —NR₇COR₁₆, —NR₇CONR₁₆R₁₆, —NR₇CO₂R₁₅, —NR₇SO₂R₁₅, —C(═N—OH)R₁₅ and Cy₂, wherein Cy₂ is optionally substituted with one or more R₁₁;
R₁₁ is chosen from C₁₋₄alkyl, haloC₁₋₄alkyl, C₁₋₄alkoxyC₁₋₄alkyl, hydroxyC₁₋₄alkyl, cyanoC₁₋₄alkyl, and R₁₄;
R₁₂ is chosen from C₁₋₄alkyl, haloC₁₋₄alkyl, C₁₋₄alkoxyC₁₋₄alkyl, hydroxyC₁₋₄alkyl, cyanoC₁₋₄alkyl, Cy₃-C₁₋₄alkyl, and Cy₂, wherein Cy₂ is optionally substituted with one or more R₁₁;
R₁₃ is chosen from hydrogen and R₁₂;
R₁₄ is chosen from halogen, —CN, —NO₂, —COR₁₈, —CO₂R₁₈, —CONR₁₈R₁₈, —OR₁₈, —OCOR₁₇, —OCONR₁₇R₁₇, —OCO₂R₁₇, —SR₁₈, —SO₂R₁₇, —SOR₁₇, —SO₂NR₁₈R₁₈, —SO₂NR₇COR₁₇, —NR₁₈R₁₈, —NR₇COR₁₈, —NR₇CONR₁₈R₁₈, —NR₇CO₂R₁₇, —NR₇SO₂R₁₇ and —C(═N—OH)R₁₇;
R₁₅ is chosen from C₁₋₄alkyl, haloC₁₋₄alkyl, C₁₋₄alkoxyC₁₋₄alkyl, hydroxyC₁₋₄alkyl, cyanoC₁₋₄alkyl, and or Cy₂, wherein Cy₂ is optionally substituted with one or more R₁₁;
R₁₆ is chosen from hydrogen, and or R₁₅;
R₁₇ is chosen from C₁₋₄alkyl, haloC₁₋₄alkyl, C₁₋₄alkoxyC₁₋₄alkyl, hydroxyC₁₋₄alkyl and cyanoC₁₋₄alkyl;
R₁₈ is chosen from hydrogen, and R₁₇;
or two R₁₇ groups or two R₁₈ groups on the same N atom are bonded together with the N atom to form a saturated 5- or 6-membered ring, which optionally contains one or two heteroatoms chosen from N, S and O and which is optionally substituted with one or more C₁₋₄alkyl groups;
Cy₁ and Cy₂ independently are chosen from a 3- to 7-membered monocyclic and a 8- to 12-membered bicyclic carbocyclic ring that is optionally saturated, partially unsaturated or aromatic, and which optionally contains from 1 to 4 heteroatoms chosen from N, S and O, wherein said ring is optionally bonded to the rest of the molecule through any available C or N atom, and wherein one or more C or S ring atoms are optionally oxidized forming CO, SO or SO₂ groups;
Cy₃ is chosen from rings (a)-(c): 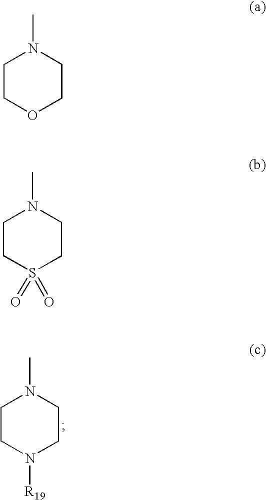
wherein R₁₉ is chosen from hydrogen, and C₁₋₄alkyl, or a salt thereof.

2. (canceled)

3. The compound according to claim 1, wherein R₁ is chosen from phenyl substituted with one or two R₃.

4. The compound according to claim 3, wherein the groups R₃ are placed at positions 3, 4 and/or 5 of the phenyl ring.

5. (canceled)

6. (canceled)

7. The compound according to claim 1, wherein:
each R₃ is independently chosen from C₁₋₄alkyl, halogen, hydroxyC₁₋₄alkyl, C₁₋₄alkoxyC₁₋₄alkyl, —OR₆, Cy₂ₐC₁₋₄alkyl, —SO₂NR₆R₆, —SO₂NR₇COR₅, —NR₆R₆, —NR₇COR₆, and Cy₁, wherein Cy₁ is optionally substituted with one or more R₉, and wherein Cy₂ is optionally substituted with one or more R₁₁;
Cy₁ is a 5- or 6-membered saturated monocyclic heterocycle which contains 1 or 2 heteroatoms chosen from N, S and O with the proviso that it contains at least one N atom, wherein said heterocycle is bonded to the rest of the molecule through a N atom, wherein one or more C or S ring atoms are optionally oxidized forming CO, SO or SO₂ groups; and
Cy₂ is a 5- or 6-membered saturated monocyclic heterocycle which contains 1 or 2 heteroatoms chosen from N, S and O, wherein said heterocycle is optionally bonded to the rest of the molecule through any available C or N atom, wherein one or more C or S ring atoms are optionally oxidized forming CO, SO or SO₂ groups.

8. (canceled)

9. (canceled)

10. The compound according to claim 7, wherein R₃ is chosen from —SO₂NR₆R₆, —NR₇COR₆, and or Cy₂C₁₋₄alkyl, wherein Cy₂ is optionally substituted with one or more R₁₁.

11. The compound according to claim 1, wherein:
R₁ is a ring of formula R_{1d}: 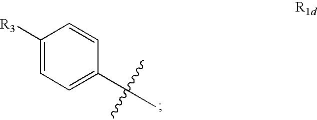
wherein R₃ is chosen from C₁₋₄alkyl, —NR₆R₆, —SO₂NR₆R₆, and Cy₁, wherein the C₁₋₄alkyl group is optionally substituted with one or more R₈ and Cy₁ is optionally substituted with one or more R₉; wherein Cy₂ is optionally substituted with one or more R₁₁; and
Cy₁ is a 5- or 6-membered saturated monocyclic heterocycle which contains 1 or 2 heteroatoms selected from N, S and O with the proviso that it contains at least one N atom, wherein said heterocycle is bonded to the rest of the molecule through a N atom, wherein one or more C or S ring atoms can be optionally oxidized forming CO, SO or SO₂ groups.

12. The compound according to claim 11, wherein
R₃ is chosen from hydroxyC₁₋₄alkyl, Cy₂C₁₋₄alkyl, —NR₆R₆, —SO₂NR₆R₆, and Cy₁, wherein Cy₁ is optionally substituted with one or more R₉ and wherein Cy₂ is optionally substituted with one or more R₁₁; and
Cy₂ is a 5- or 6-membered saturated monocyclic heterocycle which contains 1 or 2 heteroatoms chosen from N, S and O, wherein said heterocycle is optionally bonded to the rest of the molecule through any available C or N atom, wherein one or more C or S ring atoms are optionally oxidized forming CO, SO or SO₂ groups.

13. The compound according to claim 12, wherein R₃ is chosen from —SO₂NR₆R₆ and Cy₁, wherein Cy₁ is optionally substituted with one or more R₉.

14. The compound according to claim 13, wherein R₃ is chosen from formulas (i)-(iii) 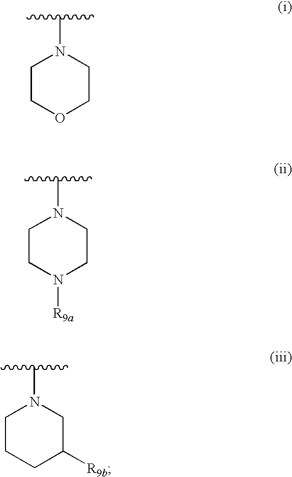
wherein R₉ₐ is chosen from hydrogen, and C₁₋₄alkyl; and
R_{9b} is chosen from hydrogen, C₁₋₄alkyl and hydroxy.

15. (canceled)

16. (canceled)

17. The compound according to claim 1 wherein R₆ in R₃ is chosen from hydrogen and R₅, wherein R₅ is C₁₋₄alkyl optionally substituted with one or more R₁₀.

18. (canceled)

19. The compound according to claim 1 wherein R₂ is chosen from phenyl and a 5- or 6-membered aromatic heterocycle bonded to the purine ring through a C atom, which is optionally fused to a 5- or 6-membered saturated, partially unsaturated or aromatic carbocyclic or heterocyclic ring, wherein R₂ optionally contains from 1 to 4 heteroatoms chosen from N, O and S, wherein the adjacent atoms to the C atom at the position of attachment to the purine ring are C atoms, wherein one or more C or S atoms of the 5- or 6-membered fused ring are optionally oxidized forming CO, SO or SO₂ groups, and wherein R₂ is optionally substituted with one or more R₄.

20. (canceled)

21. The compound according to claim 19, wherein R₂ is a 5- or 6-membered aromatic heterocycle bonded to the purine ring through a C atom, which is optionally fused to a 5- or 6-membered saturated, partially unsaturated or aromatic carbocyclic or heterocyclic ring, wherein R₂ contains from 1 to 4 heteroatoms chosen from N, O and S, wherein the adjacent atoms to the C atom at the position of attachment to the purine ring are C atoms, wherein one or more C or S atoms of the 5- or 6-membered fused ring are optionally oxidized forming CO, SO or SO₂ groups, and wherein R₂ is optionally substituted with one or more R₄.

22. (canceled)

23. The compound according to claim 21, wherein R₂ is 3-pyridyl optionally substituted with one or more R₄.

24. The compound according to claim 23, wherein R₂ is 3-pyridyl substituted with one or two R₄.

25. (canceled)

26. (canceled)

27. (canceled)

28. (canceled)

29. (canceled)

30. The compound according to claim 1, wherein
each R₄ is independently chosen from C₁₋₄alkyl, halogen, hydroxyC₁₋₄alkyl, C₁₋₄alkoxyC₁₋₄alkyl, —CONR₆R₆, —SR₆, —SOR₅, —SO₂R₅, —NR₆R₆, NR₇SO₂R₅, —NR₇CONR₆R₆, and Cy₁, wherein Cy₁ is optionally substituted with one or more R₉; and
Cy₁ is a 5- or 6-membered saturated monocyclic heterocycle which contains 1 or 2 heteroatoms chosen from N, S and O with the proviso that it contains at least one N atom, wherein said heterocycle is bonded to the rest of the molecule through a N atom, wherein one or more C or S ring atoms are optionally oxidized forming CO, SO or SO₂ groups.

31. (canceled)

32. The compound according to claim 1, wherein:
R₂ is a group of formula: 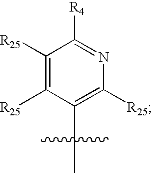
wherein each R₂₅ is independently chosen from hydrogen, halogen, and C₁₋₄alkyl.

33. (canceled)

34. (canceled)

35. The compound according to claim 32, wherein R₄ is —NR₆R₆.

36. The compound according to claim 1 wherein R₆ is C₁₋₄alkyl optionally substituted with one or more R₁₀.

37. (canceled)

38. The compound according to claim 1 wherein each R₂₅ is hydrogen.

39. (canceled)

40. (canceled)

41. (canceled)

42. (canceled)

43. (canceled)

44. A pharmaceutical composition comprising at least one compound chosen from compounds of formula I according to claim 1 or a pharmaceutically acceptable salt thereof and one or more pharmaceutically acceptable excipients

45. (canceled)

46. A method for the treatment or prevention of at least one disease chosen from transplant rejection; immune, autoimmune or inflammatory diseases; neurodegenerative diseases; and proliferative disorders, said method comprising administration of a compound of formula I: 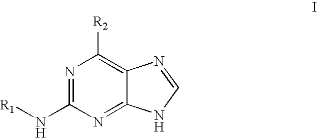
wherein:
R₁ is chosen from phenyl and a 5- or 6-membered aromatic heterocycle bonded to the NH group through a C atom, wherein the phenyl or heterocycle is optionally fused to a 5- or 6-membered saturated, partially unsaturated or aromatic carbocyclic or heterocyclic ring, wherein R₁ optionally contains from 1 to 4 heteroatoms chosen from N, O and S, wherein one or more C or S atoms of the 5- or 6-membered fused ring are optionally oxidized forming CO, SO or SO₂ groups, and wherein R₁ is optionally substituted with one or more R₃;
R₂ is chosen from phenyl and a 5- or 6-membered aromatic heterocycle bonded to the purine ring through a C atom, wherein the phenyl or heterocycle is optionally fused to a 5- or 6-membered saturated, partially unsaturated or aromatic carbocyclic or heterocyclic ring, wherein R₂ optionally contains from 1 to 4 heteroatoms chosen from N, O and S, wherein one or more C or S atoms of the 5- or 6-membered fused ring are optionally oxidized forming CO, SO or SO₂groups, and wherein R₂ is optionally substituted with one or more R₄;
R₃ and R₄ independently are chosen from C₁₋₄alkyl, C₂₋₄alkenyl, C₂₋₄alkynyl, halogen, —CN, —NO₂—COR₆, —CO₂R₆, —CONR₆R₆, —OR₆, —OCOR₅, —OCONR₅R₅, —OCO₂R₅, —SR₆, —SO₂R₅, —SOR₅, —SO₂NR₆R₆, —SO₂NR₇COR₅, —NR₆R₆, —NR₇COR₆, —NR₇CONR₆R₆, —NR₇CO₂R₅, —NR₇SO₂R₅, —C(═N—OH)R₅, and Cy₁, wherein the C₁₋₄ alkyl, C₂₋₄alkenyl and C₂₋₄alkynyl groups are optionally substituted with one or more R₈ and Cy₁ is optionally substituted with one or more R₉;
R₅ is chosen from C₁₋₄alkyl, C₂₋₄alkenyl, C₂₋₄alkynyl, and Cy₁, wherein the C₁₋₄alkyl, C₂₋₄alkenyl and C₂₋₄alkynyl groups are optionally substituted with one or more R₁₀ and Cy₂ is optionally substituted with one or more R₁₁;
R₆ is chosen from hydrogen and R₅;
R₇ is chosen from hydrogen and C₁₋₄alkyl;
R₈ is chosen from halogen, —CN, —NO₂—COR₁₃, —CO₂R₁₃, —CONR₁₃R₁₃, —OR₁₃, —OCOR₁₂, —OCONR₁₂R₁₂, —OCO₂R₁₂, —SR₁₃, —SO₂R₁₂, —SOR₁₂, —SO₂NR₁₃R₁₃, —SO₂NR₇COR₁₂, —NR₁₃R₁₃, —NR₇COR₁₃, —NR₇CONR₁₃R₁₃, —NR₇CO₂R₁₂, —NR₇SO₂R₁₂, —C(═N—OH)R₁₂, and Cy₂, wherein Cy₂ is optionally substituted with one or more R₁₁;
R₉ is chosen from R₁₄ and C₁₋₄alkyl, where the C₁₋₄alkyl is optionally substituted with one or more R₁₀;
R₁₀ is chosen from halogen, —CN, —NO₂—COR₁₆, —CO₂R₁₆, —CONR₁₆R₁₆, —OR₁₆, —OCOR₁₅, —OCONR₁₅R₁₅, —OCO₂R₁₅, —SR₁₆, —SO₂R₁₅, —SOR₁₅, —SO₂NR₁₆R₁₆, —SO₂NR₇COR₁₅, —NR₁₆R₁₆, —NR₇COR₁₆, —NR₇CONR₁₆R₁₆, —NR₇CO₂R₁₅, —NR₇SO₂R₁₅, —C(═N—OH)R₁₅, and Cy₂, wherein Cy₂ is optionally substituted with one or more R₁₁;
R₁₁ is chosen from C₁₋₄alkyl, haloC₁₋₄alkyl, C₁₋₄alkoxyC₁₋₄alkyl, hydroxyC₁₋₄alkyl, cyanoC₁₋₄alkyl, and R₁₄;
R₁₂ is chosen from C₁₋₄alkyl, haloC₁₋₄alkyl, C₁₋₄alkoxyC₁₋₄alkyl, hydroxyC₁₋₄alkyl, cyanoC₁₋₄alkyl, Cy₃-C₁₋₄alkyl, and Cy₂, wherein Cy₂ is optionally substituted with one or more R₁₁;
R₁₃ is chosen from hydrogen, and R₁₂;
R₁₄ is chosen from halogen, —CN, —NO₂—COR₁₈, —CO₂R₁₈, —CONR₁₈R₁₈, —OR₁₈, —OCOR₁₇, —OCONR₁₇R₁₇, —OCO₂R₁₇, —SR₁₈, —SO₂R₁₇, —SO₂NR₁₈R₁₈, —SO₂NR₇COR₁₇, —NR₁₈R₁₈, —NR₇COR₁₈—NR₇CONR₁₈R₁₈, —NR₇CO₇R₁₇, —NR₇SO₂R₁₇, and —C(═N—OH)R₁₇;
R₁₅ is chosen from C₁₋₄alkyl, haloC₁₋₄alkyl, C₁₋₄alkoxyC₁₋₄alkyl, hydroxyC₁₋₄alkyl, cyanoC₁₋₄alkyl, and Cy₂, wherein Cy₂ is optionally substituted with one or more R₁₁;
R₁₆ is chosen from hydrogen, and R₁₅;
R₁₇ is chosen from C₁₋₄alkyl, haloC₁₋₄alkyl, C₁₋₄alkoxyC₁₋₄alkyl, hydroxyC₁₋₄alkyl, and cyanoC₁₋₄alkyl;
R₁₈ is chosen from hydrogen, and R₁₇;
or two R₁₇ groups or two R₁₈ groups on the same N atom are bonded together with the N atom to form a saturated 5- or 6-membered ring, which optionally contains one or two heteroatoms chosen from N, S and O and which are optionally substituted with one or more C₁₋₄alkyl groups;
Cy₁ and Cy₂ independently are chosen from a 3- to 7-membered monocyclic and a 8- to 12-membered bicyclic carbocyclic ring that is optionally saturated, partially unsaturated or aromatic, and which optionally contains from 1 to 4 heteroatoms chosen from N, S and O, wherein said ring is optionally bonded to the rest of the molecule through any available C or N atom, and wherein one or more C or S ring atoms is optionally oxidized forming CO, SO or SO₂ groups;
Cy₃ is chosen from rings (a)-(c): 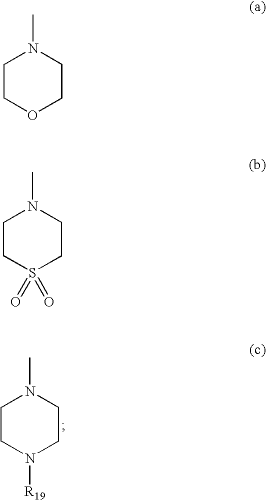
wherein R₁₉ is chosen from hydrogen, and C₁₋₄alkyl,
or a salt thereof.

47. (canceled)

48. A process for the preparation of a compound of formula I according to claim 1, which comprises:
(a) reacting a compound of formula IV with a compound of formula V 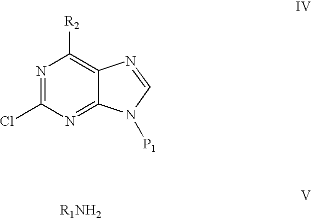
wherein R₁ and R₂ are as defined in claim 1 and P₁ is an amine protecting group, followed if required by the removal of the protecting group; or
(b) reacting a compound of formula X with a compound of formula III 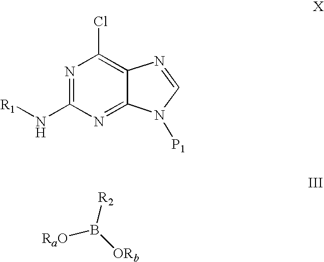
wherein R₁ and R₂ are as defined in claim 1, P₁ is an amine protecting group, and Rₐ and R_{b} are chosen from H and C₁₋₄alkyl, or Rₐ and R_{b} can be bonded forming together with the B and O atoms a 5- or 6-membered ring that can be optionally substituted with one or more methyl groups, followed if required by the removal of the protecting group; or
(c) reacting a compound of formula XV with a compound of formula XII 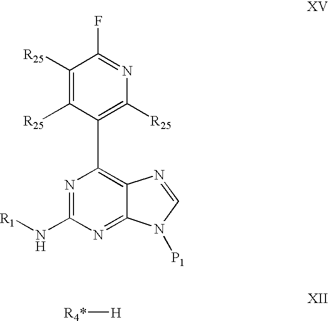
wherein R₄* is chosen from —NR₆R₆ and Cy₁ bonded through a N atom to the pyridine ring, each R₂₅ is independently chosen from hydrogen, halogen, C₁₋₄alkoxy, haloC₁₋₄alkoxy, and —SC₁₋₄alkyl, P₁ is an amine protecting group and R₁, Cy₁ and R₆ are as defined in claim 1, followed if required by the removal of the protecting group; or
(d) converting, in one or a plurality of steps, a compound of formula I into another compound of formula I.

49. The compound according to claim 1, wherein:
R₁ is a ring of formula R_{1d}: 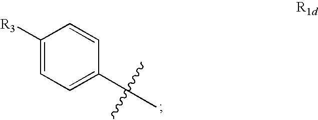
R₂ is a group of formula: 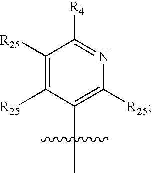
R₃ is chosen from C₁₋₄alkyl, —NR₆R₆, —SO₂NR₆R₆ and Cy₁, wherein the C₁₋₄alkyl group is optionally substituted with one or more R₈ and Cy₁ is optionally substituted with one or more R₉; R₄ is —NR₆R₆; Cy₂ₐ is optionally substituted with one or more R₁₁; each R₂₅ is independently chosen from hydrogen, halogen and C₁₋₄alkyl; and
Cy₁ is a 5- or 6-membered saturated monocyclic heterocycle which contains 1 or 2 heteroatoms chosen from N, S and O with the proviso that it contains at least one N atom, wherein said heterocycle is bonded to the rest of the molecule through a N atom, wherein one or more C or S ring atoms are optionally oxidized forming CO, SO or SO₂ groups.
